SEQUENCE LISTING (122 sequences)

SEQ ID NO: 1 - protein, 469 aa, Thermotoga maritima
  misc_feature: 6-phosphogluconate dehydrogenase
MKSHIGLIGLAVMGQNLALNIARKGYKVSVYNRTAQRTEEFVKNRVTNEEIEPHYDIESFVKSLERPRKIILMVKAGKPVDDTISQLLPHLEPGDLIIDGGNSHYMDTERRFKELSEKGILFLGMGVSGGEYGALHGPSLMPGGSREAYNLVEEILLEIAAKTEDGPCCTYVGERSAGHFVKMVHNGIEYAIMQAIAEVYHIMRDVLSLSSEEMSSIFEEWNRGELSSFLVEITYKILRKKDEETGKPMVDVILDKAEQKGTGKWTSQAALDLGIPTPSINLAVVERVISHFKDIRTRLSKLYNKRRSATQGSEEFLRDLRNSLFFAMFMAFSQGMWLIAEASKEFGYGVSLSEVLRIWKGGCIIRAKLIDTLRRYISNENAYLLENEEVMNLLKGKIDSLKNILKASIENEIPVPVLSSSYNYFMSLTEERLPANLIQAQRDFFGAHTFERVDREGVFHINWEEGEIG

SEQ ID NO: 2 - protein, 298 aa, Moorella thermoacetica
  misc_feature: 6-phosphogluconate dehydrogenase
MHIGLVGLGRMGLNLALNMLDHGHEVRGYARTKATVDKAAAQGIKGAYSLEELAGQLKTPRLVWLMIPAGKAVDEVIEQLAPLLAPGDIIVDGGNSHYRDTLRRYKFLKEKGIHLVDSGTSGGVEGARHGACCMVGAEDEVFAYLEPLFKDITVPGGYLHTGPPGSGHYVKMVHNGIEYGMMQAIGEGMEVLAGAPFKLDLKEVARVWRHGSVIRGWLMDLMEKALAKDNTLDGIKDIAYSSGEGLWTIEEALRLKVPAPVITAALLMRYRSEQEESFATKVVAALRHEFGGHDVARK

SEQ ID NO: 3 - protein, 496 aa, Thermotoga maritima
  misc_feature: glucose-6-phosphate dehydrogenase
MKCSLGLEKCPDDTLRCFPKIEQPFGIVIFGASGDLTKRKLIPALNRLFEAGILPERFFVLGAARTKMDDKKFRSRFDANPDFLEHCSYISVDYQDPESFKQLKNTIETLIKRIDSSNLVFYLAVPPDLYIPILENLSKTGLNEKPARVVIEKPFGKDLESARRLEDTLQKYFQEDQIFRIDHYLGKETVQNILVFRFANFIFEEIWNNKFVDHVQITMAEDIGVEHRAGYFENVGLLRDIFQNHMLQILALIAMEPPSSFNGENFRNERVKLLRSIRPFPVEELESWIVRGQYGRGVVNGKEVPAYREEPGVAKDSNVETFVAMKLFIDNWRWSGVPFYLRSGKRLPKKITEVAVVFKKIPHSIFAGVPSDELEPNTIVFTLQPNEGISLEFQVKRPCPGMFPQLLSMDFRYEDYFGVKLPDAYERLLLDVILGDPTLFMRRDDLEVSWELLDPVLKAWENDPVRFSPYVYPAGTWGPREADLLIERDGRKWRKL

SEQ ID NO: 4 - protein, 485 aa, Zymomonas mobilis subsp. mobilis
  misc_feature: glucose-6-phosphate dehydrogenase
MTNTVSTMILFGSTGDLSQRMLLPSLYGLDADGLLADDLRIVCTSRSEYDTDGFRDFAEKALDRFVASDRLNDDAKAKFLNKLFYATVDITDPTQFGKLADLCGPVEKGIAIYLSTAPSLFEGAIAGLKQAGLAGPTSRLALEKPLGQDLASSDHINDAVLKVFSEKQVYRIDHYLGKETVQNLLTLRFGNALFEPLWNSKGIDHVQISVAETVGLEGRIGYFDGSGSLRDMVQSHILQLVALVAMEPPAHMEANAVRDEKVKVFRALRPINNDTVFTHTVTGQYGAGVSGGKEVAGYIDELGQPSDTETFVAIKAHVDNWRWQGVPFYIRTGKRLPARRSEIVVQFKPVPHSIFSSSGGILQPNKLRIVLQPDETIQISMMVKEPGLDRNGAHMREVWLDLSLTDVFKDRKRRIAYERLMLDLIEGDATLFVRRDEVEAQWVWIDGIREGWKANSMKPKTYVSGTWGPSTAIALAERDGVTWYD

SEQ ID NO: 5 - protein, 448 aa, Clostridium aminovalericum
  misc_feature: NADH oxidase
MKIVVIGCTHAGTAAVKTILKENPEAEITIFERNDNISFLSCGIALYVGGVVKDPAGLFYSNPEELSKMGANVKIKHNVKSIDTKSKKVIAEDMNTGEEIEVSYDKLVNTTGSWPIIPPIPGIESKNILLCKNYDQANVIIRQTKDAKKIVIVGGGYIGIELVEAFQKSGKQVTLIDGLDRILNKYLDKEFTDILEDDLKKNGINLALDQCVKSFKANENGEVTSVETTKGEYEADMVILCVGFRPNNELLKGKVDMLPNDAIIVDEYMRTSDPDIFAAGDSCAVHYNPNGNYAYIPLATNAVRMGMLIGKNISTPKVKYRGTQSTSGLNLFGYNIGSTGVTVSGAPQIGLNVRSVIVKDNYRPEFMPTNEEIIMQLVYEVGTNRIVGGQVMSKYDITQSANTLSLAIQNKMTIEDLAYVDFFFQPHFDRPWNYLNILGLAALEQEGL

SEQ ID NO: 6 - protein, 392 aa, Clostridium acetobutylicum
  misc_feature: NADH oxidase
MSAEKLCENVYWVGVKDQKLRVFDIIMNTKKGSTYNSYLINDDKVAIIDTVKDGFYDEFLKSIKSVIGDKKVDYIVVQHTELDHSGSMYRLIKEYPEAKVVSSKAANMYLKEIVNDEFNSLDAMEVKELNLGKNTLEFISAPNLHWPDTMFTYNKENNILFTCDVMGCHYCPDGSIKDEGGEDYLPEMRYYFDVIMSPFKKFVNMGLDKIKDLKLDMIAPSHGPVHINDIEESVKLYREWAKEKEPKEKNVQIFYITAYGNTGIMAKHLCEDINKKGVKAEVHEITDMKMEDIVELIADANGVLVGSPTINQDAVRPVWDVLSSVCPIVNRGKAAAAFGSYGWSGEGVPMMMDRLKSLKFKTPDNGLKFKFVPASKEFSEADKFVDDFIGLL

SEQ ID NO: 7 - protein, 457 aa, Streptococcus mutans serotype c
  misc_feature: NADH oxidase
MSKIVIVGANHAGTAAINTILDNYGSENEVVVFDQNSNISFLGCGMALWIGKQISGPQGLFYADKESLEAKGAKIYMESPVTAIDYDAKRVTALVNGQEHVESYEKLILATGSTPILPPIKGAAIKEGSRDFEATLKNLQFVKLYQNAEDVINKLQDKSQNLNRIAVVGAGYIGVELAEAFKRLGKEVILIDVVDTCLAGYYDQDLSEMMRQNLEDHGIELAFGETVKAIEGDGKVERIVTDKASHDVDMVILAVGFRPNTALGNAKLKTFRNGAFLVDKKQETSIPDVYAIGDCATVYDNAINDTNYIALASNALRSGIVAGHNAAGHKLESLGVQGSNGISIFGLNMVSTGLTQEKAKRFGYNPEVTAFTDFQKASFIEHDNYPVTLKIVYDKDSRLVLGAQMASKEDMSMGIHMFSLAIQEKVTIERLALLDYFFLPHFNQPYNYMTKAALKAK

SEQ ID NO: 8 - protein, 286 aa, Thermus brockianus
  misc_feature: catalase
DRLQIELPMPKEQDPNAAAAVQALLGGRFGEMSTLMNYMYQSFNFRGKKALKPYYDLIANIATEELGHIELVSATINSLLAKNPGKDLEEGVDPVSAPLGFSKDARNAAHFIAGGANTLVMGAMGEHWHGEYVFTSGNLILDLLHNFFLEVAARTHKLRVYEMTDNPVAREMIGYLLVRGGVHAAAYGKALETLTGVEMTKMLPIPRIDNSKIPEAKKYMDLGFHRNLYRFSPSDYQDLGLIWNGASPEDGSEVVVVDGPPTGGPVFDAGHDAAEFAPEFHPAELY

SEQ ID NO: 9 - protein, 491 aa, Bacillus pumilus (Bacillus mesentericus)
  misc_feature: catalase
MTNSNHKNLTTNQGVPVGDNQNSRTAGHRGPSFLDDYHLIEKLAHFDRERIPERVVHARGAGAYGVFEVENSMEKHTRAAFLSEEGKQTDVFVRFSTVIHPKGSPETLRDPRGFAVKFYTEEGNYDLVGNNLPIFFIRDALKFPDMVHSLKPDPVTNIQDPDRYWDFMTLTPESTHMLTWLFSDEGIPANYAEMRGSGVHTFRWVNKYGETKYVKYHWRPSEGIRNLSMEEAAEIQANDFQHATRDLYDRIEKGNYPAWDLYVQLMPLSDYDELDYDPCDPTKTWSEEDYPLQKVGRMTLNRNPENFFAETEQAAFTPSALVPGIEASEDKLLQGRLFSYPDTQRHRLGANYMRIPVNCPYAPVHNNQQDGFMTTTRPSGHINYEPNRYDDQPKENPHYKESEPVLHGDRMVRQKIEKPNDFKQAGEKYRSYSEEEKQALIKNLTADLKGVNEKTKLLAICNFYRADEDYGQRLADSLGVDIRSYLQGSMK

SEQ ID NO: 10 - protein, 736 aa, Geobacillus stearothermophilus 10
  misc_feature: catalase
MKNQSQHNASKCPYHGSVTSHHSNRTTNKDWWPNQLNLSILHQHDRKTNPHDEEFNYAEEFQKLDYWALKEDLRKLMTESQDWWPADYGHYGPLFIRMAWHSAGTYRIGDGRGGASTGTQRFAPLNSWPDNANLDKARRLLWPIKKKYGNKISWADLFILAGNVAIESMGGKTIGFGGGREDVWHPEEDVYWGSEKEWLASERYSGDRELENPLAAVQMGLIYVNPEGPDGKPDPKAAARDIRETFRRMGMNDEETVALIAGGHTFGKAHGAGPATHVGPEPEAAPIEAQGLGWISSYGKGKGRDTITSGIEGAWTPTPTKWDTSYFDMLFGYEWWLTKSPAGAWQWMAVDPNEKDLAPDAEDPSKKVPTMMMTTDLALRFDPEYEKIARRFHENPDEFADAFARAWFKLTHRDMGPKTRYLGPEVPKEDFIWQDPIPKVDYELTDAEIEEIKAKILNSGLTVSELVKTAWAAASTFRNSDKRGGANGARIRLAPQKDWEVNEPERLAKVLSVYEDIQSELPKKVSIADLIVLGGSAAVEKAARDAGFDVTVPFIPGRGDATQEQTDVESFAVLEPFADGFRNYQKQEYSVPPEELLVDKAQLLGLTAPEMTVLIGGLRVLGANYCDLPHGVFTDRIGVLTNDFFVNLLDMNYEWVPTEGGIYEIRDRQTGEVRWTATRVDLIFGANSILRSYAELYAQDDNREKFVRDFINAWVKVMNADRFDIHLKQAKESVTV

SEQ ID NO: 11 - protein, 1332 aa, Bos taurus (Bovine)
  misc_feature: xanthine dehydrogenase
MTADELVFFVNGKKVVEKNADPETTLLAYLRRKLGLRGTKLGCGEGGCGACTVMLSKYDRLQDKIIHFSANACLAPICTLHHVAVTTVEGIGSTKTRLHPVQERIAKSHGSQCGFCTPGIVMSMYTLLRNQPEPTVEEIEDAFQGNLCRCTGYRPILQGFRTFAKNGGCCGGNGNNPNCCMNQKKDHTVTLSPSLFNPEEFMPLDPTQEPIFPPELLRLKDVPPKQLRFEGERVTWIQASTLKELLDLKAQHPEAKLVVGNTEIGIEMKFKNQLFPMIICPAWIPELNAVEHGPEGISFGAACALSSVEKTLLEAVAKLPTQKTEVFRGVLEQLRWFAGKQVKSVASLGGNIITASPISDLNPVFMASGTKLTIVSRGTRRTVPMDHTFFPSYRKTLLGPEEILLSIEIPYSREDEFFSAFKQASRREDDIAKVTCGMRVLFQPGSMQVKELALCYGGMADRTISALKTTQKQLSKFWNEKLLQDVCAGLAEELSLSPDAPGGMIEFRRTLTLSFFFKFYLTVLKKLGKDSKDKCGKLDPTYTSATLLFQKDPPANIQLFQEVPNGQSKEDTVGRPLPHLAAAMQASGEAVYCDDIPRYENELFLRLVTSTRAHAKIKSIDVSEAQKVPGFVCFLSADDIPGSNETGLFNDETVFAKDTVTCVGHIIGAVVADTPEHAERAAHVVKVTYEDLPAIITIEDAIKNNSFYGSELKIEKGDLKKGFSEADNVVSGELYIGGQDHFYLETHCTIAIPKGEEGEMELFVSTQNAMKTQSFVAKMLGVPVNRILVRVKRMGGGFGGKETRSTLVSVAVALAAYKTGHPVRCMLDRNEDMLITGGRHPFLARYKVGFMKTGTIVALEVDHYSNAGNSRDLSHSIMERALFHMDNCYKIPNIRGTGRLCKTNLSSNTAFRGFGGPQALFIAENWMSEVAVTCGLPAEEVRWKNMYKEGDLTHFNQRLEGFSVPRCWDECLKSSQYYARKSEVDKFNKENCWKKRGLCIIPTKFGISFTVPFLNQAGALIHVYTDGSVLVSHGGTEMGQGLHTKMVQVASKALKIPISKIYISETSTNTVPNSSPTAASVSTDIYGQAVYEACQTILKRLEPFKKKNPDGSWEDWVMAAYQDRVSLSTTGFYRTPNLGYSFETNSGNAFHYFTYGVACSEVEIDCLTGDHKNLRTDIVMDVGSSLNPAIDIGQVEGAFVQGLGLFTLEELHYSPEGSLHTRGPSTYKIPAFGSIPTEFRVSLLRDCPNKKAIYASKAVGEPPLFLGASVFFAIKDAIRAARAQHTNNNTKELFRLDSPATPEKIRNACVDKFTTLCVTGAPGNCKPWSLRV

SEQ ID NO: 12 - protein, 1333 aa, Homo sapiens (Human)
  misc_feature: xanthine dehydrogenase
MTADKLVFFVNGRKVVEKNADPETTLLAYLRRKLGLSGTKLGCGEGGCGACTVMLSKYDRLQNKIVHFSANACLAPICSLHHVAVTTVEGIGSTKTRLHPVQERIAKSHGSQCGFCTPGIVMSMYTLLRNQPEPTMEEIENAFQGNLCRCTGYRPILQGFRTFARDGGCCGGDGNNPNCCMNQKKDHSVSLSPSLFKPEEFTPLDPTQEPIFPPELLRLKDTPRKQLRFEGERVTWIQASTLKELLDLKAQHPDAKLVVGNTEIGIEMKFKNMLFPMIVCPAWIPELNSVEHGPDGISFGAACPLSIVEKTLVDAVAKLPAQKTEVFRGVLEQLRWFAGKQVKSVASVGGNIITASPISDLNPVFMASGAKLTLVSRGTRRTVQMDHTFFPGYRKTLLSPEEILLSIEIPYSREGEYFSAFKQASRREDDIAKVTSGMRVLFKPGTTEVQELALCYGGMANRTISALKTTQRQLSKLWKEELLQDVCAGLAEELHLPPDAPGGMVDFRCTLTLSFFFKFYLTVLQKLGQENLEDKCGKLDPTFASATLLFQKDPPADVQLFQEVPKGQSEEDMVGRPLPHLAADMQASGEAVYCDDIPRYENELSLRLVTSTRAHAKIKSIDTSEAKKVPGFVCFISADDVPGSNITGICNDETVFAKDKVTCVGHIIGAVVADTPEHTQRAAQGVKITYEELPAIITIEDAIKNNSFYGPELKIEKGDLKKGFSEADNVVSGEIYIGGQEHFYLETHCTIAVPKGEAGEMELFVSTQNTMKTQSFVAKMLGVPANRIVVRVKRMGGGFGGKETRSTVVSTAVALAAYKTGRPVRCMLDRDEDMLITGGRHPFLARYKVGFMKTGTVVALEVDHFSNVGNTQDLSQSIMERALFHMDNCYKIPNIRGTGRLCKTNLPSNTAFRGFGGPQGMLIAECWMSEVAVTCGMPAEEVRRKNLYKEGDLTHFNQKLEGFTLPRCWEECLASSQYHARKSEVDKFNKENCWKKRGLCIIPTKFGISFTVPFLNQAGALLHVYTDGSVLLTHGGTEMGQGLHTKMVQVASRALKIPTSKIYISETSTNTVPNTSPTAASVSADLNGQAVYAACQTILKRLEPYKKKNPSGSWEDWVTAAYMDTVSLSATGFYRTPNLGYSFETNSGNPFHYFSYGVACSEVEIDCLTGDHKNLRTDIVMDVGSSLNPAIDIGQVEGAFVQGLGLFTLEELHYSPEGSLHTRGPSTYKIPAFGSIPIEFRVSLLRDCPNKKAIYASKAVGEPPLFLAASIFFAIKDAIRAARAQHTGNNVKELFRLDSPATPEKIRNACVDKFTTLCVTGVPENCKPWSVRV

SEQ ID NO: 13 - protein, 1331 aa, Rattus norvegicus (Rat)
  misc_feature: xanthine dehydrogenase
MTADELVFFVNGKKVVEKNADPETTLLVYLRRKLGLCGTKLGCGEGGCGACTVMISKYDRLQNKIVHFSVNACLAPICSLHHVAVTTVEGIGNTQKLHPVQERIARSHGSQCGFCTPGIVMSMYTLLRNQPEPTVEEIENAFQGNLCRCTGYRPILQGFRTFAKDGGCCGGSGNNPNCCMNQTKDQTVSLSPSLFNPEDFKPLDPTQEPIFPPELLRLKDTPQKKLRFEGERVTWIQASTMEELLDLKAQHPDAKLVVGNTEIGIEMKFKNMLFPLIVCPAWIPELNSVVHGPEGISFGASCPLSLVESVLAEEIAKLPEQKTEVFRGVMEQLRWFAGKQVKSVASIGGNIITASPISDLNPVFMASGAKLTLVSRGTRRTVRMDHTFFPGYRKTLLRPEEILLSIEIPYSKEGEFFSAFKQASRREDDIAKVTSGMRVLFKPGTIEVQELSLCFGGMADRTISALKTTPKQLSKSWNEELLQSVCAGLAEELQLAPDAPGGMVEFRRTLTLSFFFKFYLTVLQKLGRADLEDMCGKLDPTFASATLLFQKDPPANVQLFQEVPKDQSEEDMVGRPLPHLAANMQASGEAVYCDDIPRYENELSLRLVTSTRAHAKITSIDTSEAKKVPGFVCFLTAEDVPNSNATGLFNDETVFAKDEVTCVGHIIGAVVADTPEHAQRAARGVKITYEDLPAIITIQDAINNNSFYGSEIKIEKGDLKKGFSEADNVVSGELYIGGQEHFYLETNCTIAVPKGEAGEMELFVSTQNTMKTQSFVAKMLGVPDNRIVVRVKRMGGGFGGKETRSTVVSTALALAAHKTGRPVRCMLDRDEDMLITGGRHPFLAKYKVGFMKTGTVVALEVAHFSNGGNTEDLSRSIMERALFHMDNAYKIPNIRGTGRICKTNLPSNTAFRGFGGPQGMLIAEYWMSEVAITCGLPAEEVRRKNMYKEGDLTHFNQKLEGFTLPRCWDECIASSQYLARKREVEKFNRENCWKKRGLCIIPTKFGISFTLPFLNQGGALVHVYTDGSVLLTHGGTEMGQGLHTKMVQVASRALKIPTSKIHISETSTNTVPNTSPTAASASADLNGQGVYEACQTILKRLEPFKKKKPTGPWEAWVMDAYTSAVSLSATGFYKTPNLGYSFETNSGNPFHYFSYGVACSEVEIDCLTGDHKNLRTDIVMDVGSSLNPAIDIGQVEGAFVQGLGLFTMEELHYSPEGSLHTRGPSTYKIPAFGSIPIEFRVSLLRDCPNKRAIYASKAVGEPPLFLASSIFFAIKDAIRAARAQHGDNAKQLFQLDSPATPEKIRNACVDQFTTLCVTGVPENCKSWSVRI

SEQ ID NO: 14 - protein, 1203 aa, Escherichia coli (strain K12)
  misc_feature: xanthine dehydrogenase/xanthine oxidase
MRVDAIAKVTGRARYTDDYVMAGMCYAKYVRSPIAHGYAVSINDEQARSLPGVLAIFTWEDVPDIPFATAGHAWTLDENKRDTADRALLTRHVRHHGDAVAIVVARDELTAEKAAQLVSIEWQELPVITTPEAALAEDAAPIHNGGNLLKQSTMSTGNVQQTIDAADYQVQGHYQTPVIQHCHMESVTSLAWMEDDSRITIVSSTQIPHIVRRVVGQALDIPWSCVRVIKPFVGGGFGNKQDVLEEPMAAFLTSKLGGIPVKVSLSREECFLATRTRHAFTIDGQMGVNRDGTLKGYSLDVLSNTGAYASHGHSIASAGGNKVAYLYPRCAYAYSSKTCYTNLPSAGAMRGYGAPQVVFAVESMLDDAATALGIDPVEIRLRNAAREGDANPLTGKRIYSAGLPECLEKGRKIFEWEKRRAECQNQQGNLRRGVGVACFSYTSNTWPVGVEIAGARLLMNQDGTINVQSGATEIGQGADTVFSQMVAETVGVPVSDVRVISTQDTDVTPFDPGAFASRQSYVAAPALRSAALLLKEKIIAHAAVMLHQSAMNLTLIKGHIVLVERPEEPLMSLKDLAMDAFYHPERGGQLSAESSIKTTTNPPAFGCTFVDLTVDIALCKVTINRILNVHDSGHILNPLLAEGQVHGGMGMGIGWALFEEMIIDAKSGVVRNPNLLDYKMPTMPDLPQLESAFVEINEPQSAYGHKSLGEPPIIPVAAAIRNAVKMATGVAINTLPLTPKRLYEEFHLAGLIMFDFASYHRAATLADAINLLADNPQAKLLAGGTDVLIQLHHHNDRYRHIVDIHNLAELRGITLAEDGSLRIGSATTFTQLIEDPITQRHLPALCAAATSIAGPQIRNVATYGGNICNGATSADSATPTLIYDAKLEIHSPRGVRFVPINGFHTGPGKVSLEHDEILVAFHFPPQPKEHAGSAHFKYAMRDAMDISTIGCAAHCRLDNGNFSELRLAFGVAAPTPIRCQHAEQTAQNAPLNLQTLEAISESVLQDVAPRSSWRASKEFRLHLIQTMTKKVISEAVAAAGGKLQMNHSETITIECTINGMPFQLHAAPGTPLSELLREQGLLSVKQGCCVGECGACTVLVDGTAIDSCLYLAAWAEGKEIRTLEGEAKGGKLSHVQQAYAKSGAVQCGFCTPGLIMATTAMLAKPREKPLTITEIRRGLAGNLCRCTGYQMIVNTVLDCEKTK

SEQ ID NO: 15 - protein, 1405 aa, Blastobotrys adeninivorans
  misc_feature: xanthine dehydrogenase/xanthine oxidase
MVDLQLHQSLGHHNFTNKLTFYVNGVKRTISNPDPRGTLLDFIRTHEGLTGTKLGCSEGGCGACTVVVASWDREQGEIIYSAVNSCIVPLVAVEGKHLITVEGIGSSNNPHPAQERIALFHGSQCGFCTPGIVMSLYALLRNTGGQPSKEQIAESFDGNLCRCTGYKPIIDAANTFSCGRPGGCCRDNASGKANGAGATVGNGMANGAAAVANGNGAAANGCCKGNGAANGCCKSNGSAATTANGDDKEVDMNKLFTPNGLPLKPYSAKTELIFPPALKKYELNPLFFGNEQKVWFRPVTKLQLLQIKHAYPESKIVGGASEIQIEIKMKAANYNISVYANDIEELKTHKYIPGKGLEFGANISLSKLEEVCDKLVHELDPNVSQIYGAILEQLKYFAGRQIRNAATPAGNIATASPISDLNPVLVAAEAVLTVESIENGEEQISMTDFFVGYRKTKLPAHGVITKIFVPETVPRNEVVMAYKQAKRKDDDIAIVTACLRLALDDDFRISKARLAYGGVGPFTTAAKGTAEFLTGKLLRRETAKEVLEGAIDCLIKEFDLPYSVPGGMAAYRRTLIMSFFYKFYSTVLEKIGLAGEAQDNSALENTYDPQALLEVTRKHPVGSRDLTNPYEQRIVGKSDPHLSALKQVTGEAVYIDDIPPYHGECFGVQVMSTKPRARILSVDPSPALEVEGVVGYVDVNDLPSREANIWGPTPVGKEPFFADGEVYYVGQCIGVIIATDRMIAEEAARLVKVEYEELETVITIEEAIEAQSFFDYQPKAEKGDVDGAFAESAYTFEGTSRIGSQEHFYLETQGSLVVPEPEDGEMKVYSSSQNPTETQVFVAQATGVPSSRIVARVKRLGGGFGGKESRCCHLSSIAAVAAKKYKRPVRMILSRSEDMLTAGQRHPFVMKWKVGLDKNYKFTALEAKLYANAGWSMDLTKGVIERAVLHAENCYDFPNARIQGIPCRTSVASNTAFRGFGGPQGMFMAECYIYEIADQLGIEPDTLREINYLVPGVSSTPFKQAITEDFTVPDMVKQIKKQSNYDDLRRQVEEFNSKHKWIKRGLAHVPTMFGISFGATFLNQAGALVHIYHDGSILLTHGGTEMGQGLHTKMAMVCAEELKVPLSQVFISETSTNTVPNTSASAASASSDLNGMAVKHACDQLNERLAPYRERLGENATMEQLAHAAYFDRVNLSANGFYKTPDIGFVWGDPNPKPAFFYFTQGCAVAMVEVNTLTGDWSNLRTDIVMDIGRPINQAIDYGQIEGAFVQGQGLFTIEESLWLRNGALFTRGPGAYKIPGFRDIPQEFNVGHLRDRPFKHLKTIHRSKGIGEPPLFLGSSVFFAIRDALSYARRQNLGEATMPAGLVAPMTTERIRMLAGDSLYEHKGKIEPTEGDDKPFFVNA

SEQ ID NO: 16 - protein, 855 aa, Clostridium thermocellum
  misc_feature: alpha-glucan phosphorylase
MYLFGKITVTAVIPDELSKLKDIAYNLWWSWNSEAIDLFREIDLALWEKLGKNPVRFLKEVSQKKLEAKLKDPDYMQRYKKVVNDFETYMNETDTWFSRNFPDKKDHMVAYFSAEYGLNEVLPIYSGGLGVLSGDHCKSASDLGIPFTAIGLFYKEGYFSQRINSEGWQETIFTPLNPSNLPIQPALNDKGEEVIISVELPGRVVYAKVWVVKVGRVNLYLMDTDIEQNSPYDRGLTARLYGGDQETRIQQEIFLGIGGARVLDALGIKATVYHMNEGHSAFLGLELIRKLVQNHNLPFNQAKEVVASSVIFTTHTPVPAGNDVFPLEMIDRYFGNYWPSLGINRHEFLDLGLRIGEHHNFNMTVLALTLAGQKNGVSELHGAVSRNIFKNVWPGIPEDEIPIGHITNGIHTLTWLSPSIKYLYDKYLDKDWKERLHEKEVWEKVDDIPDEELWKTHCVLKTKMIGYVREKLKEQRAANGESIERIKEVDTLLDSNALTIGFARRFATYKRANLIFRDLARIQKLLNNPEKPVQIIFAGKAHPADGPAHEIIKYINDIAKQEGFNGKVILVENYNMTLARNLVQGVDIWLNNPRRPLEASGTSGQKVAINGIINFSVLDGWWCEGYNGKNGWAIGDDTFYDNEYHQDNADSESIYNILEKQIIPTFFDRNEKGVPEKWVKIMKESIKSIAAQYSTHRMVQDYINKYYIPAMERYDKIKASNYQFAANISEWKKKVAHLWPQVQIIAEKTANQLKERNFISGESIPIYATVNLGGLEPSDVKVQAYYGSIGKNNSIENPVIVDMDVVERNSDGTYLYSANITLYEGGEYGYTFRVIPNHPDIINPFDLGLIRWIVQ

SEQ ID NO: 17 - protein, 823 aa, Thermotoga neapolitana
  misc_feature: alpha-glucan phosphorylase
MLKKLPENLEHLEELAYNLWWSWSRPAQRLWRKIDPEGWEEHRNPVKILKEVSDERLEELSKDDDFISLYELTIERFKDYMEKEDTWFNVNYPEWDEKIVYMCMEYGLTKALPIYSGGLGILAGDHLKSASDLGLPLIAIGLLYKHGYFTQQIDRDGKQIEIFPDYNPEDLPMKPLKDEKGNQVIVEVPLDSTVVKARVFEVKVGRVSLYLLDPDIEENEERYRKICNYLYNPEPDVRVSQEILLGIGGMKLLRALNLKPGVIHLNEGHPAFSSLERIKNYMEEGYSFTEALEIVRQTSVFTTHTPVPAGHDRFPFDLVEKKLSKFFEGFEKRNLLMDLGKDETGSFNMTYLALRTSSFINGVSKLHAEVSRRMFKNVWQGVPVEEIPIEGITNGVHMGTWINREMRKLYDRYLGRVWRDHTDLEGIWYGVDRIPDEELWQAHLRAKKRFIEYIKESVRRRNERLGIDEDVPNIDENSLIIGFARRFATYKRAVLLLSDLERLKKILNDPERPVYVVYAGKAHPRDDAGKEFLKRIYEVSQMPEFKNRIIVLENYDIGMARLMVSGVDVWLNNPRRPMEASGTSGMKAAANGVLNASVYDGWWVEGYNGRNGWVIGDESVLPETEVDDPRDAEALYDLLENEIIPTYYENKEKWIFMMKESIKSVAPRFSTTRMLKEYTEKFYIKGLVNKEWLERKENAERFGAWKERILRNWSSVSIERIVLEDTRSVEVTVKLGDLSPDDVLVELLIGRGESMEDLEIWKVIQIRKHRREGDLFIYSYVNGALGHLGSPGWFYAVRVLPYHPKLPTRFLPEIPVVWKKVLG

SEQ ID NO: 18 - protein, 830 aa, Thermococcus kodakarensis
  misc_feature: alpha-glucan phosphorylase
MVNVSNAVEDEIRKKLPYPLGDLADLAYNYWWSWNRRATKLWEYIDPEHWREYKNPVKLLLDTPEERFKELVRDDDFMNLYELVMDQFHAYMNPRSTWFSTNYPKWDKPIVYLCMEYGISKSLPIYSGGLGILAGDHVKTASDLGLPFIAIGLLYKHGYFRQEIDKDGRQVEIFPEYKPEEMPIKPVLDREGKPLRIEVPIGEGIVYARAFEVNVGRVKLYLLDTDVPENSPDDRTICDYLYNAEMDKRIKQEILLGIGGMRLLKALEIEPGVIHLNEGHPAFANLQRIAWYMEEGLTFTEALSIVRGTTVFTTHTPVPAGHDRFPIEEVKKRLAKFLEGREELLELGREGDQLNMTLLAIRTSSYVNGVSQLHAEVSKRMWKDLWPGVPLNEIPIEGITNGVHTMTWVHNEMRKLFDRYIGKVWREHTNLEGIWYAVERIPDEELWEAHLQAKREFIDLLRRKAIERNKRLGVDDPIPNIDENALIIGFARRFATYKRATLLLTDLERLKKIVNNPERPVYIVFGGKAHPRDDGGKEFLRRVYEVSKMPEFRGKIFVMENYDMGSARLMVAGVDVWLNNPRRPMEASGTSGMKAGLNGVLNVSIYDGWWVEGYNGKNGWVIGEETTEPETEADDPKDAESLYTLLEEEVIPTYYENRAKWIYMMKESIKSIAPRFSTHRMVKEYMDRFYSKAMSNYIWLTRENYSGAKEIAAWKDRVRASWDKVEIRDAKAEGNRLEVRVYLDGLKPEDVEVELYYGVRAQGYEIEKPHIVELRHPEQVGEGEWLYTYEGNALRHLGDPCWHYAVRVHPHHEKLPHRFLLGLVKWKGLD

SEQ ID NO: 19 - protein, 504 aa, Bifidobacterium adolescentis [sequence omitted]

SEQ ID NO: 20 - protein, 490 aa, Leuconostoc mesenteroides
  misc_feature: sucrose phosphorylase
MEIQNKAMLITYADSLGKNLKDVHQVLKEDIGDAIGGVHLLPFFPSTGDRGFAPADYTRVDAAFGDWADVEALGEEYYLMFDFMINHISRESVMYQDFKKNHDDSKYKDFFIRWEKFWAKAGENRPTQADVDLIYKRKDKAPTQEITFDDGTTENLWNTFGEEQIDIDVNSAIAKEFIKTTLEDMVKHGANLIRLDAFAYAVKKVDTNDFFVEPEIWDTLNEVREILTPLKAEILPEIHEHYSIPKKINDHGYFTYDFALPMTTLYTLYSGKTNQLAKWLKMSPMKQFTTLDTHDGIGVVDARDILTDDEIDYASEQLYKVGANVKKTYSSASYNNLDIYQINSTYYSALGNDDAAYLLSRVFQVFAPGIPQIYYVGLLAGENDIALLESTKEGRNINRHYYTREEVKSEVKRPVVANLLKLLSWRNESPAFDLAGSITVDTPTDTTIVVTRQDENGQNKAVLTADAANKTFEIVENGQTVMSSDNLTQN

SEQ ID NO: 21 - protein, 488 aa, Thermoanaerobacterium thermosaccharolyticum
  misc_feature: sucrose phosphorylase
MALKNKVQLITYPDSLGGNLKTLNDVLEKYFSDVFGGVHILPPFPSSGDRGFAPITYSEIEPKFGTWYDIKKMAENFDILLDLMVNHVSRRSIYFQDFLKKGRKSEYADMFITLDKLWKDGKPVKGDIEKMFLRRTLPYSTFKIEETGEEEKVWTTFGKTDPSEQIDLDVNSHLVREFLLEVFKTFSNFGVKIVRLDAVGYVIKKIGTSCFFVEPEIYEFLDWAKGQAASYGIELLLEVHSQFEVQYKLAERGFLIYDFILPFTVLYTLINKSNEMLYHYLKNRPINQFTMLDCHDGIPVKPDLDGLIDTKKAKEVVDICVQRGANLSLIYGDKYKSEDGFDVHQINCTYYSALNCDDDAYLAARAIQFFTPGIPQVYYVGLLAGVNDFEAVKKTKEGREINRHNYGLKEIEESVQKNVVQRLLKLIRFRNEYEAFNGEFFIEDCRKDEIRLTWKKDDKRCSLFIDLKTYKTTIDYINENGEEVKYLV

SEQ ID NO: 22 - protein, 811 aa, Clostridium thermocellum
  misc_feature: cellobiose phosphorylase
MKFGFFDDANKEYVITVPRTPYPWINYLGTENFFSLISNTAGGYCFYRDARLRRITRYRYNNVPIDMGGRYFYIYDNGDFWSPGWSPVKRELESYECRHGLGYTKIAGKRNGIKAEVTFFVPLNYNGEVQKLILKNEGQDKKKITLFSFIEFCLWNAYDDMTNFQRNFSTGEVEIEGSVIYHKTEYRERRNHYAFYSVNAKISGFDSDRDSFIGLYNGFDAPQAVVNGKSNNSVADGWAPIASHSIEIELNPGEQKEYVFIIGYVENKDEEKWESKGVINKKKAYEMIEQFNTVEKVDKAFEELKSYWNALLSKYFLESHDEKLNRMVNIWNQYQCMVTFNMSRSASYFESGIGRGMGFRDSNQDLLGFVHQIPERARERLLDLAATQLEDGGAYHQYQPLTKKGNNEIGSNFNDDPLWLILATAAYIKETGDYSILKEQVPFNNDPSKADTMFEHLTRSFYHVVNNLGPHGLPLIGRADWNDCLNLNCFSTVPDESFQTTTSKDGKVAESVMIAGMFVFIGKDYVKLCEYMGLEEEARKAQQHIDAMKEAILKYGYDGEWFLRAYDDFGRKVGSKENEEGKIFIESQGFCVMAEIGLEDGKALKALDSVKKYLDTPYGLVLQNPAFTRYYIEYGEISTYPPGYKENAGIFCHNNAWIICAETVVGRGDMAFDYYRKIAPAYIEDVSDIHKLEPYVYAQMVAGKDAKRHGEAKNSWLTGTAAWNFVAISQWILGVKPDYDGLKIDPCIPKAWDGYKVTRYFRGSTYEITVKNPNHVSKGVAKITVDGNEISGNILPVFNDGKTHKVEVIMG

SEQ ID NO: 23 - protein, 980 aa, Clostridium thermocellum
  misc_feature: cellodextrin phosphorylase
MITKVTARNNKITPVELLNQKFGNKINLGNFADRVFTDAAFKNVAGIANLPMKAPVMQVLMENCIVSKYLKQFVPDRSVCFVEEGQKFYIVLEDGQKIEVPEDVNKALRATVSDVKHWAGYLTEDGEHVIALLKPAPGPHFYVNLLIGNRLGFKRTLQTTPKSVVDRFGRGSFRSHAATQVLATRFDMRQEENGFPANRQFYLYEDGKQIFYSALIDDNIVEATCKHSCNRTVIKYKTACNLEITRTIFLVPHKKGFPLATELQRIEIKNASDKARNLSITYTGMFGTGAVHAIFEDVTYTNVIMQSAALYNDKGEFIGITPDYYPEEFKQDTRFVTMIVRNGDEKSFPQSFCTDYNDFVGTGTLEHPAADVIEQQAEPQRSGILCPGCAVYVEPGKTVIIDTFTGLSSSKDNENYSDAVMLRELDNLLRYFEKSESVEETLNEIINFHENYGKYFQFNPGNKLFDSGFNRNLALEVLYQTFMICSFGQTQKGYREIGSGNSGLFASMYYFINIGYQDFVKELLFEWTANVYKMGYANHNFYLVGKQRDCIPMTACLLQAYYRYIIYTKDTSVLNEEVPVADGNNEKRAVRETLKAIIQYSACISVGDHGLPLLDLADWNDCLKIDSNSIDGATKEKLYYEQLKKTNGKYGDRFMSDYSESVMNAFLLKLAIDHLAEIATLDNDTQLAQQMSELSKEVTDRIQKHAWKENFFARVLINRYKDGSYTYLGAKGDKLSADPNIDGVYFLNSFAWSVLSDVATDEQIAIMVDVIKKYLLTPYGLRLVTPADLNKIANDTATGHYFFGDRENGAVFKHASMMAVVALIKAAKKVKDNELAKEMARIAYFMIDLVLPYKTEIRSRLQEIQGYALNISILTQEKILTFVEPGTATWLNLNLISLAGIEYTRDGISFNPILREEETQLNFTLKAPKCSYKFSITKPVGFARMESSEYELFVDGQKIDNTVIPMYTDEKEHIVTLKFK

SEQ ID NO: 24 - protein, 1019 aa, Thermosipho africanus
  misc_feature: cellodextrin phosphorylase/cellodextrin phosphorylase
MKKFDFVIENYSKQKLFSSFLPGIAGKNGIPLWVFYVNRGQCIASFGIENKANSILEFKPAGQSHTDTPLKGFRTFIKVDGRYYEPFSELTNFKREMRINKNSLEIEERNNELGLKVKVIYFVLPNEDFAALVRRVEIENEDKYEKHIEIIDGLPEVIPYGVSNGLYKEMGYTARAWMHVYNYEKKVPFYSVRTTIGDLEIVEEINNGYFYFASSGDELLEVIYDKNVLFGNSTSLQVPLVFKELGIKEVLRKEQYDENLLPSAFGVLERKLKDKVVINSMIGFSKEKGLINNNINTLKKDEYIISKKEEADLIVEELVSEIKTKTSNKLFDKYCEQNYLDNVLRGGYPLVFENKDGKVVYHIYSRKHGDLERDYNFFVLEAKKYSQGNGNFRDVAQNRRNDVIFHPEIEDFNLSMFVNLIQADGNNPLVVKGTRFKFEGDPSILDGVNEELKEFILNNYFTPGEILEKLKDKNVNEDEFVSKILYHSSQHEQAEFGEGYWIDHWTYLMDLVDTYKEIYPDKLQKTLFEKFEYKIYDSHAYVKPRKEKYKLYKGKVRQIAAVGESHEKLKIIQERGHNYLTDRNGNIYKTNMFEKLLLLAVNKFATLDPYGMGLEMEANKPGWNDALNGLPALFGSGMSETFELKRLINFMYEELKKYNKDIEVFEELQEFIQKIKVELENYFNDNDQFIYWDNVSNAKEEYRSKVFYSITGNKKKISKVELLAILKKMIKKLDEGIERAKNYGKGIYPTFFTYELVDYEVIDGVIIPKKFEVNVLPYFLEGIVRAFKVIDKDEKKKLYDFVKNSNIYDKKLKMYKTSESILNQPYSIGRIRAFTPGWLENESVFMHMEFKYLLELIKSDMLEEFYEDIKTALPPYMDYKVYGRSILENSSFIVSSANSNPNLHGQGFYARLSGSTAEFLSMWKYMFIGDKLFTLENNELTFTFEPKINKEFFENGVIEFKLFSKTKVKYVNPQLKEKIGRIEVFVDGKKFEIHGNKIKGELAHKLRNKKIDEVICYFE

SEQ ID NO: 25 - protein, 282 aa, Cellulomonas sp.
  misc_feature: nicotinamide riboside phosphorylase
TTTTPPSTPPLDDPATDPFLVARAAADHIAQATGVEGHDMALVLGSGWGGAAELLGEVVAEVPTHEIPGFSAPAVAGHLSVTRSIRVERADGSVRHALVLGSRTHLYEGKGVRAVVHGVRTAAATGAETLILTNGCGGLNQEWGAGTPVLLSDHINLTARSPLEGPTFVDLTDVYSPRLRELAHRVDPTLPEGVYAQFPGPHYETPAEVRMAGILGADLVGMSTTLEAIAARHCGLEVLGVSLVTNLAAGISPTPLSHAEVIEAGQAAGPRISALLADIAKR

SEQ ID NO: 26 - protein, 289 aa, Bos taurus (Bovine)
  misc_feature: nicotinamide riboside phosphorylase
MANGYTYEDYQDTAKWLLSHTEQRPQVAVICGSGLGGLVNKLTQAQTFDYSEIPNFPESTVPGHAGRLVFGILNGRACVMMQGRFHMYEGYPFWKVTFPVRVFRLLGVETLVVTNAAGGLNPNFEVGDIMLIRDHINLPGFSGENPLRGPNEERFGVRFPAMSDAYDRDMRQKAHSTWKQMGEQRELQEGTYVMLGGPNFETVAECRLLRNLGADAVGMSTVPEVIVARHCGLRVFGFSLITNKVIMDYESQGKANHEEVLEAGKQAAQKLEQFVSLLMASIPVSGHTG

SEQ ID NO: 27 - protein, 289 aa, Homo sapiens (Human)
  misc_feature: nicotinamide riboside phosphorylase
MENGYTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

SEQ ID NO: 28 - protein, 277 aa, Escherichia coli (strain K12) [sequence omitted]

SEQ ID NO: 29 - protein, 272 aa, Bacillus halodurans
  misc_feature: Purine nucleoside phosphorylase
MLNVTQLQEATTFIQQQIETKPTIGLILGSGLGILADEIEQPVKVPYSDIPHFPVSTVQGHAGQLVIGMLEGKQVIAMQGRFHFYEGYSLEVVTFPVRVMKALGVEQIIVTNAAGGVNESFEAGDLMIIRDHINNMAQNPLIGPNDEAFGVRFPDMSNAYSERLRTLAKEKGNTLNLKLQEGVYVANTGPVYETPAEVRMIRKLGGDAVGMSTVPEVIVARHAGLEVLGISCISNMAAGILPQPLSHDEVIETTERVRQDFLNLVKAIVKDM

SEQ ID NO: 30 - protein, 275 aa, Thermus thermophilus
  misc_feature: Purine nucleoside phosphorylase
MPDMELYDKIQEAVAYVRSKTDFVPEVGLVLGSGLGPLADEVEKVAEIPYGEIPHFPVSTAPGHAGRLVLGRLEGKPVLVYKGRVHYYEGYSAEEVVFPVRVGFFLGARTFLLTSAAGGLNPRFRAGGIMLHLDYINFAGANPLRGPNDERLGPRFPVMFEAYDPELIELARKVARRQDLHLFEGVYAWFMGPSFASRAELRLLRELGADAIGMSTVPEVIALRHLGARVLGLSTITDMAVPEREHHATEEEVLRVAAETGPVFRRYVRGILAEL

SEQ ID NO: 31 - protein, 265 aa, Thermotoga maritima
  misc_feature: Purine nucleoside phosphorylase
MMKKIEEARTFISERTNLSPDILIILGSGFGPFIEKVEDPVIIDYKDIPHFPQPTVEGHSGKLVFGRISDKPVMIMAGRFHLYEGHDPATVAFPVYLAKYVGVKGVVVTNAAGAINPEFKPGEIILVRDIINFMFRNPLRGPNDEKIGPRFPDMSSVVDPEWARKIQERLSLKEGVYIGVLGPSYETPAEIRVFEKLGADLVGMSTVPEVIAAKHCGLKVVVFSCVTNMAAGITHGRLSHEEVVRTTKMAQGKIEKALTTAVEVF

SEQ ID NO: 32 - protein, 275 aa, Meiothermus silvanus
  misc_feature: Purine nucleoside phosphorylase
MDGLGIYEAIQQTIAFIRQQTDFVPEVGIVLGSGLGPLADEIETVASFSYADLPHFPRSTAPGHEGRLILGRLEGKNVLGYKGRVHYYEHYTAAEVVFPVRVGFYLGAKTFFLTSAAGGLNPNWQAGDLMLHLDYLNMAGANPLKGPNDERMGPRFPVMFDAYDPELNTLARKVARAQDLNLREGVYVWFAGPAFASRAELRMLRLLGADAIGMSMVPEVIALRHLGARVLGLSTITDMALPDSTHHATEQEVLEVAARTGATFRKFVRGILAAM

SEQ ID NO: 33 - protein, 275 aa, Clostridium thermocellum
  misc_feature: Purine nucleoside phosphorylase
MDNIYEKARETASFIKRIIKETPEIAIVLGSGLGPLADEIENKVEIDYKDVPNFPVTTVEGHAGKFVYGILENRRVIAMKGRFHHYEGYDVSQIVFPVRVFKMLGINNLIVTNASGGINRSFRPGDLMIIKDHISFFAPSPLRGKNIDEFGLRFPDMCKAYNPKLVEICKKAASDVGVDVKEGVYAFTQGPMYETPAEIRALGILGADAVGMSTVPEVIAARHANMNILGISCITNMAAGILDQPLTHEEVMKTAKEAENKFVRLVKRVISVWEV

SEQ ID NO: 34 - protein, 236 aa, Geobacillus thermodenitrificans
  misc_feature: Purine nucleoside phosphorylase
MSVHIGAKPGEIAEHILLPGDPLRAKYIAETFLEGAVCYNEVRGMLGFTGTYKGHRISVQGTGMGVPSISIYVNELIQSYGVKTLIRVGTCGAIQPDVRVRDVILAMSASTDSNMNRLIFRGRDYAPTADFHLLRTAYDVGMEKGLALKVGNVFTADMFYNDEPNWETWARYGVLAVEMETAALYTLAAKFDCRALSVLTVSDHIITGEETTAEERQTTFNDMIEVALEAASRNDA

SEQ ID NO: 35 - protein, 261 aa, Deinococcus geothermalis
  misc_feature: Purine nucleoside phosphorylase
MVARVPARPFASPPATLDRVSVHLNARPGEIAETVLLPGDPLRAQHIAETFFENPVQHNSVRGMLGFTGTYRGVPVSVQGTGMGIASSMIYVNELIRDYGCQTLIRVGTAGSYQPDVHVRDLVLAQAACTDSNINNIRFGLRNFAPIADFELLLRAYQMARDRGFATHVGNILSSDTFYQDDPESYKLWAQYGVLAVEMEAAGLYTLAAKYGVRALTILTISDHLVTREETTAEERQTTFNGMIEVALDAALGLAVPSNSM

SEQ ID NO: 36 - protein, 230 aa, Giardia intestinalis
  misc_feature: Hypoxanthine/guanine phosphoribosyltransferase (HGPRT)
MICSVTGKPVKDVLSTFFKDRNDVLESEVKKFHLLATFEECKALAADTARRMNEYYKDVAEPVTLVALLTGAYLYASLLTVHLTFPYTLHFVKVSSYKGTRQESVVFDEEDLKQLKEKREVVLIDEYVDSGHTIFSIQEQIKHAKICSCFVKDVDAIKKHSALADTKMFYGYTPMPKGSWLIGFGLDDNGLRRGWAHLFDINLSESEVTEFRRRLTEHIKGLNINGVNRY

SEQ ID NO: 37 - protein, 221 aa, Trypanosoma cruzi
  misc_feature: Hypoxanthine/guanine phosphoribosyltransferase (HGPRT)
MPREYEFAEKILFTEEEIRTRIMEVAKRIADDYKGKGLRPYVNPLVLISVLKGSFMFTADLCRALSDFNVPVRMEFICVSSYGEGVTSSGQVRMLLDTRHSIEGHHVLIVEDIVDTALTLNYLYHMYFTRRPASLKTVVLLDKREGRRVPFSADYVVANIPNAFVIGYGLDYDDTYRELRDIVVLRPEVYAEREAARQKKQRAIGSADTDRDAKREFHSKY

SEQ ID NO: 38 - protein, 241 aa, Trypanosoma cruzi
  misc_feature: Hypoxanthine/guanine phosphoribosyltransferase (HGPRT)
MFIRLFAREEEGEKEGKICHMPREYEFAEKILFTEEEIRTRIKEVAKRIADDYKGKGLRPYVNPLVLISVLKGSFMFTADLCRALCDFNVPVRMEFICVSSYGEGLTSSGQVRMLLDTRHSIEGHHVLIVEDIVDTALTLNYLYHMYFTRRPASLKTVVLLDKREGRRVPFSADYVVANIPNAFVIGYGLDYDDTYRELRDIVVLRPEVYAEREAARQKKQRAIGSADTDRDAKREFHSKY

SEQ ID NO: 39 - protein, 218 aa, Gallus gallus
  misc_feature: Hypoxanthine/guanine phosphoribosyltransferase (HGPRT)
MATHSPCIVIGDDEQGYDLDLFCIPKHYADDLEKVYIPHGLIMDRTERLAREIMKGMGGHHIVALCVLKGGYKFFADLLDYIKALNRNSDKSIPMTVDFIRLKSYCNDQSTGDIKVIGGDDLSTLTGKNVLIVEDIIDTGKTMKTLLSLLKQYNPKMVKVASLLVKRTPRSVGYRPDFVGFEVPDKFVVGYALDYNEYFRDLNHICVISETGKQKYKA

SEQ ID NO: 40 - protein, 184 aa, Clostridium thermocellum
  misc_feature: Hypoxanthine/guanine phosphoribosyltransferase (HGPRT)
MINQIKEILVTREELKNNAKELGKRISSDYEGKELVLIGVLKGGVVFFADLIREITIPIDVDFISVSSYGNSTKSSGVVRIIKDIDIDITNKHVLIVEDLVDTGLTLHYLKSMFEARGPKDVKICTALDKPSRRKVDLEIDYKGITIPDKFVVGYGLDYAEKYRNLPDVCVLDSSVYTDKEDMD

SEQ ID NO: 41 - protein, 237 aa, Haloferax volcanii
  misc_feature: Hypoxanthine/guanine phosphoribosyltransferase (HGPRT)
MNRAEKAALQLQAVSVLRMLKETRTYDELAELTGLPAGDLNRYVNGHVLPGVERAREVVEGVGRDALADELEARVRFDDEGYVDNSGVVFDQSFLDLVAPVAANALDLERPDVILTAATDGITLGAAMASYFGARLAYAKKSKETAVEDFIESRQRLASGIELTYYLPASAIDDGERVLVVDDLIRSGETQELLLDIALQADADIAGVFALIAVGDEGTNRARELTDAPVGALSSFD

SEQ ID NO: 42 - protein, 188 aa, Haloferax volcanii
  misc_feature: Hypoxanthine/guanine phosphoribosyltransferase (HGPRT)
MDQLRQSLLDAPIIEKGDYEYFVHPISDGVPMLEPGLLREIVIKIIRKANLENVDKIVTPAAMGIHISTAVSLMTDIPLVVIRKREYGLDGEVSLHQQTGYSEGDMFINDVNEGDRVLVLDDVLSTGGTMKAVLDALDHIGADVVDTVAIIKKAGPNELDDSDHHVKTLINVTVEDGEVVIVDEHGDD

SEQ ID NO: 43 - protein, 181 aa, Haloferax volcanii
  misc_feature: Hypoxanthine/guanine phosphoribosyltransferase (HGPRT)
MQRLEESLHEAPIIDKDGYSYLVHPISNGVPMLDPQLLREVVVGITRAADLDVDKIVAPEAMGIHIATALSLQTDVPLVVIRKREYGLDGEVALHQTTGYSESEMFINDIEDGDRVLVVDDLLSTGGTLAAICGALDDIGAEVSDIVVAIRKVGETALDDTDYEATSLVDISVDEDGVEIH

SEQ ID NO: 44 - protein, 188 aa, Halobacterium salinarum
  misc_feature: Hypoxanthine/guanine phosphoribosyltransferase (HGPRT)
MDRLKQSLLDAPIIEKDGYHYFVHPISDGVPMLEPELLREIVIRIIRKAELDEVDKIVTPAAMGIHISTAVSLMTDIPIVVIRKREYGLPGEVALSQETGYSENEMYINDVHEGDRVLVLDDVLSTGGTLRAITDALEQTGADVADVLAVIKKAGPNELDDTDMDVRTLINVDVADGEVVVVDDQGDH

SEQ ID NO: 45 - protein, 239 aa, Halobacterium salinarum
  misc_feature: Hypoxanthine/guanine phosphoribosyltransferase (HGPRT)
MNRAEKATLQLQAVDVLRTLKETRTYDELSADTGLPAGDLNRYVNGHVLPSADRAREVVQNAGADLLQAELAARVHVDDDGYVDNSGIVFDQSFLELVAPVAAETFDFDAPDVVLTAATDGITLAAALAGYYGADCAYAKQSKETAVEEFVEARQRLESGIEFTYYLPASALDDGDTVLVVDDLIRSGETQELLLDIADAAGADVTGVFALIAAGETGVDRARERTDAPVGALTDLDDL

SEQ ID NO: 46 - protein, 495 aa, Haemophilus ducreyi
  misc_feature: Nicotinamide phosphoribosyltransferase (NAMPT)
MDNLLNYSSRASAIPSLLCDFYKTSHRIMYPECSQIIYSTFTPRSNEQAPYLTQVVSFGFQAFIIKYLIHYFNDNFFSRDKHDVVTEYSAFIEKTLQLEDTGEHIAKLHELGYLPIRIKAIPEGKTVAIKVPVMTIENTHSDFFWLTNYLETLINVSLWQPMTSASIAFAYRTALIKFANETCDNQEHVPFQSHDFSMRGMSSLESAETSGAGHLTSFLGTDTIPALSFVEAYYGSSSLIGTSIPASEHSVMSSHGVDELSTFRYLMAKFPHNMLSIVSDTTDFWHNITVNLPLLKQEIIARPENARLVIRPDSGNFFAIICGDPTADTEHERKGLIECLWDIFGGTVNQKGYKVINPHIGAIYGDGVTYEKMFKILEGLQAKGFASSNIVFGVGAQTYQRNTRDTLGFALKATSITINGEEKAIFKNPKTDDGFKKSQKGRVKVLSRDTYVDGLTSADDFSDDLLELLFEDGKLLRQTDFDEIRQNLLVSRTTL

SEQ ID NO: 47 - protein, 490 aa, Haemophilus ducreyi
  misc_feature: Nicotinamide phosphoribosyltransferase (NAMPT)
MYLNPVTAIDGYKVDHRRQYPDNTQVIFSNLTARKSRRGYTDQMVFFGLQYFIKHYLIDSWNRDFFQQPKEQVICQFSRRINNYLGPNNVGTQHIEELHDLGYLPIKIMALPEGSVYPLKVPCLILYNTDERFFWLTNYLETILSANVWGMCTSATTALQYRKIFEAYALETDGDIAFVDWQGHDFSFRGMYGVEAAIMSGAAHLLSFTGTDTIPAIDFLEQYYLADSDKELVGGSVPATEHSVMCAGGMENELETFRRLIEDIYPTGIVSIVSDSWDFWQVMTEFTLALKDRILARDGKVVFRPDTGCPVKIICGDPQAPIGSPEYKGAIECLWDVFGGSTTAKGYKLLDSHVGLIYGDSITIERAEAICAGLKAKGFASTNIVFGIGSFTYQHVTRDTDGYAVKATFAKVDGKDREIFKDPKTDDGTKKSAKGLVAVFKDEQGQFYLKDQASWQDVNNCEFVPVFADGELLTEYSLADIRARLAASRR

SEQ ID NO: 48 - protein, 491 aa, Homo sapiens (Human) [sequence omitted]

SEQ ID NO: 49 - protein, 463 aa, Meiothermus ruber
  misc_feature: Nicotinamide phosphoribosyltransferase (NAMPT)
MKTLNPHNLILNTDSYKASHFAQFPKGMTYASWYIESRGGDSNFVRFFGLQAFLIEYLSKGVSLADVEEAQEVFLAHGLPFPTEGWRYIAQDLGGRLPVRIRAVPEGKVVPVHNPLVIIESTDPKVPWLPGWLETALLRAVWYPTTVCTVSWGIRNTIKEYLEKTADDPEAELPFKLHDFGARGVSSLESAGLGGMAHLVNFMGTDTVTALIYARNYYGAEMAGYSIPAMEHSTVTSFGRTGEAQAYRQMLETFAKPGALMAMVIDSYNREHAVGQIIGEELRELIQQSGATVVIRPDSGDPPFVVLRTVQTLEAKFGATLNRKGYKVLNGVRVIQGDGVNADSIRKVLFLLEQWGYSASNVAFGMGGALLQHPHRDTQKFAQKLHLVTVNGETYGVGKSPVDDPGKLSKKGRLDVIQDERGIRTVELPLEAAQPHPQSILQTVFENGSITRRYTWEEVRNNA

SEQ ID NO: 50 - protein, 485 aa, Tenacibaculum maritimum
  misc_feature: Nicotinamide phosphoribosyltransferase (NAMPT)
MNPLLYTDGYKVDHRRQYPNQTTLVYSNWTPRKSRIQGIENVVFFGLQYFIKKYILEDFENNFFKQPKKIICEKYNRRINNYLGDNAVGIKHIEALHDLGYIPMVIKALPEGASVPIKVPMFTMYNTLPNFFWLTNYFETLLSTTIWLPCNSATIAKEYRKILDNYAEETSSTPELVDWQGHDFSMRGMGGLEAAIVSAAGHLLSFTGTDTIPAIDFLETYYHADSNTELVGGSVPATEHSVMCMGTNIGEQETFKRLITEVYPSGIVSIVSDTWDLWKVLTEYLPNLKEEVVARNGKVVIRPDSGDPVAIICGNPNGKTNAEKKGVIQLLWDTFGGSTNTKGYKELDGHIGAIYGDSITLERATEICERLKQKGFASTNVVLGIGSFTYQYNTRDTFGFAMKATYGEVAGKGREIFKDPITDDGTKKSAKGLLKVEKENGIYKLYDQVSWNEEKKGALKEVFRDGKLLSDQTLTDIRDRIKNTI

SEQ ID NO: 51 - protein, 488 aa, Marivirga tractuosa
  misc_feature: Nicotinamide phosphoribosyltransferase (NAMPT)
MNRNNLILLADAYKYSHHKLYYPGTNKVYSYLESRGGKFNSTVFYGLQYFIKAYLEGIAFTQQDLDEAIPILKEVFGRDDVFDPSKFQYILDKYNGKLPIRIKAVPEGTEVPVSNVLLTIENTDPNCFWLTNFLESLLMQVWYPITVATISKEIKKTLTKFYSETASNGAEAGIEYVLNDFGLRGVSSVESAGLGGSAHLVNFQGSDTIIGSVLAQKYYNTSTIYGKSVPATEHSICTLLGEKGELDIFKHVLNSFPTGIVACVSDSYNIFKACEEYWGEDLKTQIMEREGVLVIRPDSGDPIKTLLRIFDILFEKFGYIINDRGYKVLPPQVRVIQGDGVNYDAIKNIFQALKENKISAENLVLGMGGALLQKLDRDTQKFALKCSYAEINDEGVIVQKSPTEMDENGNITESFKKSKGGRLKLIKSEGAFKTVNEHEYPEIEDELVTVFENGEIIQDWTFEQVRANANNTLNDKQYDLKSNIQSIT

SEQ ID NO: 52 - protein, 462 aa, Synechocystis sp. (strain PCC 6803 / Kazusa)
  misc_feature: Nicotinamide phosphoribosyltransferase (NAMPT)
MNTNLILDVDSYKVSHWLQYPPDTTAMYSYVESRGGRYPVTVFFGLQYILKRYLTQSIEPWMVEEANRLLTAHGLPFNYGGWRYIAEDLQGRLPVRIKAVPEGSVIPVHNVLMTVESTDPKVFWLVSWLETLLMRVWYPITVATQSWHLKQRIYQSLCRTADDPDGEINFKLHDFGARGVSSGESSGIGGLAHLVNFQGSDTVKALVYGQQYYNCPMAAYSIPAAEHSTITAWGREGEVLAYENMLTQFAKPGSVLAVVSDSYDLWNAIDHLWGDHLRAQVLDSGATVVIRPDSGDPVAIVAQTLERLEACFGSTLNSKGFRVLNAVRVIQGDGVDEESISAILEKTESLGFSTTNLAFGMGGALLQKVNRDTQKFAMKCSEVTVEDKAIPVYKDPVTDPGKTSKKGRLSLVKTDSGYGTVPTSSEDLLQVVYENGHLLQDQCLDAIRQRAWPLIRVNVPAS

SEQ ID NO: 53 - protein, 309 aa, Escherichia coli (strain K12)
  misc_feature: Ribokinase
MQNAGSLVVLGSINADHILNLQSFPTPGETVTGNHYQVAFGGKGANQAVAAGRSGANIAFIACTGDDSIGESVRQQLATDNIDITPVSVIKGESTGVALIFVNGEGENVIGIHAGANAALSPALVEAQRERIANASALLMQLESPLESVMAAAKIAHQNKTIVALNPAPARELPDELLALVDIITPNETEAEKLTGIRVENDEDAAKAAQVLHEKGIRTVLITLGSRGVWASVNGEGQRVPGFRVQAVDTIAAGDTFNGALITALLEEKPLPEAIRFAHAAAAIAVTRKGAQPSVPWREEIDAFLDRQR

SEQ ID NO: 54 - protein, 315 aa, Thermoanaerobacterium thermosaccharolyticum
  misc_feature: Ribokinase
MKNILVVGSINMDIVIKVEHIPKIGETVIAKEVNYSFGGKGANQAVAIAKLGGNVTMIGRVGDDNFGKELIENLDKQKVKPSGIEIDKTEKTGMAFIYVSDKGENNISVNQGANKKLDVEQINRHLDLFDAAEICVLQLEIPIETVKYAIEICNKKNIKVILNPAPARKIPEEILKNIYILTPNESELSILTKKNVKTIEDINNASEILLESGVQNIITTLGEKGCFLKNKNDSLHFAAVQVDAIDTTAAGDSFTGAISIALNNGKSIKEAIEFATYVSALTVTKEGTQDSLPDKLEVEKFINERREVHEKNNFA

SEQ ID NO: 55 - protein, 306 aa, Thermus thermophilus
  misc_feature: Ribokinase
MRFFVLGDVSVDLLFFLERIPEPGEEVPSRRALMKPGGAGGTLAAQLASLGHRVFLAGRVGKDPFAELALSRVREVGVDLRHLQEDPEHTTSSVLILVVPGGERAMVSAEGASRYLDPALFKPRFLDQVDAVVLSAYALVGGPSRSYAAEVLEAARRRELPVFADLGAGAVRAAGKELLKHLRGVGWLLMNEGELKALTGASSISQGVARLRQEGFQHLAVKVGAMGSIVVTPEGEELIEPFPVEDIVDSTGAGDAYTAAFAHAILEGLSPVEAGRLANLAGALAATAIGAQGRLVTLEDLKVAAG

SEQ ID NO: 56 - protein, 492 aa, Thermotoga maritima
  misc_feature: D-xylulokinase
MNLYVGLDVGTTGVKGILVNEKGEILATANERLTMFTPQPAWAEQDPLSWWEAVKKILKNLSERSKEMGGKIRAISTSGQMHSLVAIDDNGKVLRNAILWCDQRTYKECEEATQILGGEENVLKLVGNPILPGFTLPKILWIRKHEPEIYGKISKIMLPKDFINYMLTGEVKTEHSDASGTVMYSVSKMEWNKDVLKELNIPESVLPEIIPSNGVVGNVKPEVASDLGLSEDTLVIGGGADNACAALGIAVVEPGDVMVSLGTSGTVLAPTKGNQPDPKGRVHFFAHTVPETRYHMGVMLSATYSLEWFKEKFLSEDYETINEEVDKIPAGSNGIIFLPYLNGERTPHRDPFARGVFFGISSYNTKWDMVRAIFEGVAFGIKDSFDILRELKVVLNSVRITGGGSKSRVWNKMLADMTGLRIQKPAVDEGASYGAAILAVSGSMGENPAKISKEWFRVKSYTDPAVENTETYEKLHEKFKKLYTSLKEMFRS

SEQ ID NO: 57 - protein, 523 aa, Geobacillus stearothermophilus 10
  misc_feature: D-xylulokinase
MAIHGDVVIGIDIGTTSTKAVVFDGRGKVLASHAVDYPIIQPHPGFAEQDPDELFAAVIQAVGAVTVRYGIRPEQVKAMGLSAAMHSIMALDESGRPLTRLLIWADNRSVAQAERLLKEQNGLAIYRRTGTPIHPMSPLSKLLWLKEEQPDVFQQAHRFVSVKDYVLYRLYGDYVADHSMASATGLFRLDTLDWDEEVLALIGIRRDQLPRLVPATHVMTGMRREWAEKMGVAPDVPVVIGASDGVLANVGVGAVLPGEAAITIGTSGAVRTIAATPTTDEKGRTFCYALTPGYWVVGGPTNNGGILLRWLRDEFGAQEREVAKKLGMDPYDLLTKYAERVPPGSEGLVFLPFLSGERAPYWNANARGTFFGLGLHHKREHFIRAVMEGVCFSILSVALAIRDVTGPMTEIRVSGGFAKSPFWRQMLSDMLGKPLIVPQTHEASALGAAAVALYALGELPSLETVKTWIDTTARHEPKEEHTLIYAELFDLYARLYERLKEEFNIIAGFQRRNGEKGSETKSV

SEQ ID NO: 58 - protein, 348 aa, Myceliophthora thermophila
  misc_feature: Adenosine kinase
MASTKDYRLLCLENPLLDIQAFGNEALLQKYGLKANDAILAEPKHLDIYEDLLNNYDAKLIAGGAAQNTARGAQYILPPNSVVYLGGAGDDKYAAILRDACKQAGLRVEYRVDPKIPTGRCGVVITGHNRSMCTDLGAANHYDLEHLKRPDIWALVENAEVFYVGGYHFTVCPPAIMELANQAATKNKPFIVSLSAPFIPQFFKEPLDASAPYWDYVIGNETEAEAYADSHNLGTKDVKEIAKALANLPKVNTQRKRVAVITQGTEPTIVAVQGEDEVKEYPVHAISKEEICDTNGAGDAFAGGFCAGIVEGRPLDVSIDMGQWLASLSIRELGPSYPFPKKTYPGKQ

SEQ ID NO: 59 - protein, 347 aa, Thermothielavioides terrestris
  misc_feature: Adenosine kinase
MTSTKEYRLLCLENPLLDIQAFGNEALLEKYGLKANDAILAEEKHLSIYEDLLNNYDAKLIAGGAAQNTARGAQYMLPPNSVVYLGGAGDDKYAAILRDACKQAGLRVEYRVDPKIPTGRCGVVITGHNRSMCTDLGAANHYDLDHLKRPDIWALVENAEAFYVGGYHFTVCPPAIMELANQAATKNKPFILSLSAPFIPQFFKEPLDASAPYWDYVIGNETEAEAYANSHGLGTKDVKEIAKALANLPKVNTQRKRVAVITQGTEPTVVAVQGEDEVKEYPVHEIPKEEINDTNGAGDAFAGGFCAGIVEGRPLDECVDMGQWLARLSIKELGPSYPFPKQTYSRQ

SEQ ID NO: 60 - protein, 340 aa, Saccharomyces cerevisiae (strain ATCC )
  misc_feature: Adenosine kinase
MTAPLVVLGNPLLDFQADVTAEYLAKYSLKENDAILVDAKSGDAKMAIFDELLQMPETKLVAGGAAQNTARGAAYVLGAGQVVYFGSVGKDKFSERLLNENEKAGVKSMYQVQNDIGTGKCAALITGHNRSLVTDLGAANFFTPDHLDKHWDLVEAAKLFYIGGFHLTVSPDAIVKLGQHAKENSKPFVLNFSAPFIPHVFKDALARVLPYATVIIANESEAEAFCDAFQLDCANTDLEAIAQRIVKDSPVEKTVIFTHGVEPTVVVSSKGTSTYPVKPLDSSKIVDTNGAGDAFAGGFMAGLTKGEDLETSIDMGQWLAALSIQEVGPSYPSEKISYSK

SEQ ID NO: 61 - protein, 240 aa, Saccharomyces cerevisiae
  misc_feature: Nicotinamide riboside kinase
MTSKKVILVALSGCSSSGKTTIAKLTASLFTKATLIHEDDFYKHDNEVPVDAKYNIQNWDSPEALDFKLFGKELDVIKQTGKIATKLIHNNNVDDPFTKFHIDRQVWDELKAKYDSINDDKYEVVIVDGFMIFNNTGISKKFDLKILVRAPYEVLKKRRASRKGYQTLDSFWVDPPYYFDEFVYESYRANHAQLFVNGDVEGLLDPRKSKNIKEFINDDDTPIAKPLSWVCQEILKLCKD

SEQ ID NO: 62 - protein, 348 aa, Myceliophthora thermophila
  misc_feature: Nicotinamide riboside kinase
MDPPRTITIGISGCSSSGKTTLARLLRDMFPETFILHEDDFYKAESELPIKDGFADWDCPESISIPDLEAALAHVRATGTFPPNVNSLEDLNTVGPCPATPDQIAACAAKVRAWLSPNRPGALIFPPKPPSSSSSPPPPPSSSSSLLPSSSSSSDATRPPKTRVCILDGFLLYSPPPSTPTPTSTSTAAAAAAAVSSPLRGVMAQLDVKLFLKASKAKALERRGARDGYVHVDGFWKDPPGYVERVVWPNYVEAHRWLFERGDVEGGRLDRAVLEREGILTPGLDPDPDGGEAGGESGGGGGEEGGVGVGVGGNQDIEFGKILEWAVEVVMRELERLCLGEKDGVGKE

SEQ ID NO: 63 - protein, 173 aa, Pseudomonas alcaligenes
  misc_feature: Nicotinamide riboside kinase
MKVVVLTGPESSGKTFLAERLQQRFGGLVVGEYVRHFIDQEQRETCLADIPAIARGQLEWEDAARAQAPGLLILDTHLLSNILWSRTLFGDCPDWLEPTLLERHYDLHLLLSPEGVAWQGDGQRCQPDLAERLRFHQDCADWLQRHARPWVAVHGDWLQREARVIEQVARLIG

SEQ ID NO: 64 - protein, 289 aa, Clostridium thermocellum
  misc_feature: NAD kinase
MLKIGIIPNEDKDEELKYTRILVDSIKKCGGTAIVCDDIALKLGDKESNINEDNIVDMSDVMVCLGGDGTFLKAARMTVVKGKPLLGVNLGKLGFLADVDKNDIENAVKRLVEDKFTVDERMMLDTVIVRDGKIIAEDIVLNDVVISRGAISRILHLKTYINDAFMDLYPGDGLIISTPTGSTAYSLSAGGPLVEPDVDLIICTPICPHLLYSRSFITTADRVIKVVVAESSSHEAMVTVDGQNGYEVRGGDVIITKKSRIRMPMVRLNGKNFFDVLRGKIYDRGESMK

SEQ ID NO: 65 - protein, 258 aa, Thermotoga maritima
  misc_feature: NAD kinase
MKIAILYREEREKEGEFLKEKISKEHEVIEFGEANAPGRVTADLIVVVGGDGTVLKAAKKAADGTPMVGFKAGRLGFLTSYTLDEIDRFLEDLRNWNFREETRWFIQIESELGNHLALNDVTLERDLSGKMVEIEVEVEHHSSMWFFADGVVISTPTGSTAYSLSIGGPIIFPECEVLEISPIAPQFFLTRSVVIPSNFKVVVESQRDINMLVDGVLTGKTKRIEVKKSRRYVRILRPPEYDYVTVIRDKLGYGRRIE

SEQ ID NO: 66 - protein, 278 aa, Thermococcus kodakarensis
  misc_feature: NAD kinase
MKFGVVARRDKLEALKLAYRVYDFLKVSGFDVVVDEDTYRYLGEFSEDDVLPLEEFDVDIIVVIGGDGTILRVEHKTKKEIPILGINMGTLGFLTEVEPHETFFALSRVIEGDYHIDERIKLRTFLDGENRVPDALNEVAVLTGIPGKIIHLKYYIDGGLADEVRADGLIISTPTGSTGYAMSAGGPFVDPRLDVVVIAPLAPIALSSRPMVVPSSSRIDVRNVAMTREVILSVDGQFYTYLSPETEITIVRSPRKTKFVRFNREIYPKYTMKIKSRF

SEQ ID NO: 67 - protein, 277 aa, Pyrococcus horikoshii
  misc_feature: NAD kinase
MKFGIVARRDKEEALKLAYRVYDFLKVHGYEVVVDKETYEHFPHFKEGDVIPLDEFDVDFIVAIGGDGTILRIEHMTKKDIPILSINMGTLGFLTEVEPSDTFFALNRLIEGEYYIDERIKVRTYIDGENRVPDALNEVAILTGIPGKIIHMKYYVDGGLADEVRADGLVVSTPTGSTGYAMSAGGPFIDPRLDVILIAPLLPLPKTSVPMVIPGSSRIDIRMLTDREIILAIDGQYYEHLPPNVEITVVKSPRKTKFIRFTREIYPKYTIRIKERH

SEQ ID NO: 68 - protein, 272 aa, Thermoanaerobacterium saccharolyticum
  misc_feature: NAD kinase
MRTTRSLVEWIIQHGYKPILNEVVAQKIGFFEYGKSGTEIFENSDFIVALGGDGTILNVARQCASFSTPILGVNLGHLGFLAEVDAENVIEAVEKIVNDEFFIDKRMMLEASIIKENMEAVNLIALNDIVVTRGSFSRMVKLKVFVNEQYVNTYLADGIIISSPTGSTAYSLSAGGPIVYPNLELFVITPICPHTLHSRSIIVSEKDKVKLVIVGENQDVMATTDGQQGYKLNSGDTIYVKKSNRYTNLIRLKSMNFFELLRSKLSERNFNI

SEQ ID NO: 69 - protein, 362 aa, Micrococcus luteus
  misc_feature: NAD kinase
MPYTPGRRILVLTHTGREDAISAALQATRMFAEEGLVTVMLEQDVAAIRAAAGDPPEFAPETLGVDCELEDITLGLVLGGDGSVLRAADFVRGYNVPLLAVNLGHVGFLAESERTDLHRTVQAIASESYVVIERMALDVVVHVEGREVARTWALNEASVEKSHRERMLEVVVSVDNSPLTSFGCDGVVLATPTGSTAYAFSAGGPVVWPSVEALLCVPISAHALFTRPLVVGPRSTIGVDVLTRTRETGVLWCDGRRTVELPPQARVEVSRSAEPVRLARLNPTPFAERLVRKFRLPTDGWRGPVTAQERAGVLHEVETVEPRHAGPRPDVVEPATMPLSVPSPADIQRHRDRGARHPGEDA

SEQ ID NO: 70 - protein, 262 aa, Thermobifida fusca
  misc_feature: polyphosphate glucokinase
MASRGRVGLGIDIGGSGIKGAPVDLDRGTFVVDRVKIATPQPATPEAVAAVVAEIVTAFADDVPQDAPLGVTFPAVIQHGVARSAANVDRSWIGTNVEELLSAVTGRRVLVVNDADAAAMAEHRYGAASGVDGVVLLTTLGTGIGTAVLVDGVLLPNTEFGHLEIDGYDAETRASASAKERENLSYKEWAEERLQRYYSVIEDLLWPDLIVVGGGVSRKADKFLPHLRLRAPIVPAKLRNTAGIVGAAVLAAERLGGDRVSA

SEQ ID NO: 71 - protein, 396 aa, Geobacillus stearothermophilus
  misc_feature: acetate kinase
MAKVLAINAGSSSLKFQLFEMPAETVLTKGVVERIGFDDAIFTIVVNGEKHQEVTAIPDHAAAVKMLLDKLIRYGIIRSFDEIDGIGHRVVHGGEKFSDSVLITEDVIKQIEEVSELAPLHNPANLVGIRAFQEVLPDVPAVAVFDTAFHQTMPEQSFLYSLPYEYYTKFGIRKYGFHGTSHKYVTQRAAELLGRPIEQLRLISCHLGNGASIAAVEGGKSIDTSMGFTPLAGVAMGTRSGNIDPALIPYIMEKTGMTADEVIDVLNKKSGMLGISGISSDLRDLEKAAAEGNERAELALEVFANRIHKYIGSYAARMCGVDAIIFTAGIGENSEVVRAKVLRGLEFMGVYWDPILNKVRGKEAFISYPHSPVKVLVIPTNEELMIARDVMRLANL

SEQ ID NO: 72 - protein, 408 aa, Methanosarcina thermophila CHTI-55
  misc_feature: acetate kinase
MKVLVINAGSSSLKYQLIDMTTESALAVGLCERIGITDSIITQKRFDGKKLEKTTDFPNHKVAMEEVVKSLTDPEFGVIKDMSEINAVGHRVVHGGEKFTSSALIDEDVEKSIEECFDLAPLHNPPNMMGIKACQELMPGVPMVAVFDTAFHMTIPKYAYMYALPYELYEKYGIRKYGFHGTSHMYVSRRAIAMLGKPAEETKIITCHLGNGSSITAVKGGKSVDTTMGFTPLEGVCMGTRCGSIDPAVVPFIMEKENLSTREIDTLMNKKSGVLGISGVSNDFRDLDEAASKGNERANLALEIFAYKVKKVIGEYTAVLDGVDAIVFTAGIGENSASIRKRILSGLENFGIKIDEEKNKIRGQEIDISTPDSKVRVFVIPTNEELTIARDTKEIVETEAKLRSSIPV

SEQ ID NO: 73 - protein, 403 aa, Thermotoga maritima
  misc_feature: acetate kinase
MRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDIEEAALKGDEWCKLVLEIYDYRIAKYIGAYAAAMNGVDAIVFTAGVGENSPITREDVCSYLEFLGVKLDKQKNEETIRGKEGIISTPDSRVKVLVVPTNEELMIARDTKEIVEKIGR

SEQ ID NO: 74 - protein, 306 aa, Corynebacterium glutamicum
  misc_feature: polyphosphate kinase
MVGKLPIMAETNENDLPVIDLAQIEGYVVDDSDEDDPVLLRPDGTPIETWREDFPYEERVTREDYEKVKRSLQIELLKWQNWTKETGQRHIILFEGRDAAGKGGTIKRFNEHLNPRGARTVALEKPSPRESTSWYFQRYIQHFPAAGEIVFFDRSWYNRSGVERVMGFCTESQHAEFLREVPMLENMILGSGISLTKFWFSVTRKEQRTRFAIRQVDPVRQWKLSPMDLASLDRWDDYTRAKEEQFRYTDTDESPWITIKSNDKKRARINAMRYVLSKFDYTDKDYELVGEPDPKVVLRGRDQIGD

SEQ ID NO: 75 - protein, 320 aa, Corynebacterium glutamicum
  misc_feature: polyphosphate kinase
MRKKKDGQNLPDFRKNPPKLDKKAYEKELKRLQAELVDLQQWVVETGARVVIVMEGRDAAGKGSAIKRITQYLNPRSARIEALPTPNSREKGQWYFQRYIEKLPTAGEIVIFDRSWYNRAGVERVMGFCTSQEYRRFLHQAPIFERLLVEDGIHLRKYWFSVSDEEQIERFEDRLSDPLRRWKLSPMDLQSITRWEDYSRAKDEMFIHTDIPSAPWYTVESEDKKRSRINVISHLLSTIPYEKIDRPLPEIPHRPDSESDYVRPPRDEFRYVPDVAAHLEEERIKKEEKAKKAKKPAKAAGKNSDKQKSSGGKGKKKSKK

SEQ ID NO: 76 - protein, 305 aa, Cytophaga hutchinsonii
  misc_feature: polyphosphate kinase
MATDFSKLSKYVETLRVKPKQSIDLKKDFDTDYDHKMLTKEEGEELLNLGISKLSEIQEKLYASGTKSVLIVFQAMDAAGKDGTVKHIMTGLNPQGVKVTSFKVPSKIELSHDYLWRHYVALPATGEIGIFNRSHYENVLVTRVHPEYLLSEQTSGVTAIEQVNQKFWDKRFQQINNFEQHISENGTIVLKFFLHVSKKEQKKRFIERIELDTKNWKFSTGDLKERAHWKDYRNAYEDMLANTSTKQAPWFVIPADDKWFTRLLIAEIICTELEKLNLTFPTVSLEQKAELEKAKAELVAEKSSD

SEQ ID NO: 77 - protein, 266 aa, Deinococcus radiodurans
  misc_feature: polyphosphate kinase
MDIDNYRVKPGKRVKLSDWATNDDAGLSKEEGQAQTAKLAGELAEWQERLYAEGKQSLLLILQARDAAGKDGAVKKVIGAFNPAGVQITSFKQPSAEELSHDFLWRIHQKAPAKGYVGVFNRSQYEDVLVTRVYDMIDDKTAKRRLEHIRHFEELLTDNATRIVKVYLHISPEEQKERLQARLDNPGKHWKFNPGDLKDRSNWDKFNDVYEDALTTSTDDAPWYVVPADRKWYRDLVLSHILLGALKDMNPQFPAIDYDPSKVVIH

SEQ ID NO: 78 - protein, 267 aa, Meiothermus ruber
  misc_feature: polyphosphate kinase
MKKYRVQPDGRFELKRFDPDDTSAFEGGKQAALEALAVLNRRLEKLQELLYAEGQHKVLVVLQAMDAGGKDGTIRVVFDGVNPSGVRVASFGVPTEQELARDYLWRVHQQVPRKGELVIFNRSHYEDVLVVRVKNLVPQQVWQKRYRHIREFERMLADEGTTILKFFLHISKDEQRQRLQERLDNPEKRWKFRMGDLEDRRLWDRYQEAYEAAIRETSTEYAPWYVIPANKNWYRNWLVSHILVETLEGLAMQYPQPETASEKIVIE

SEQ ID NO: 79 - protein, 266 aa, Deinococcus geothermalis
  misc_feature: polyphosphate kinase
MQLDRYRVPPGQRVRLSNWPTDDDGGLSKAEGEALLPDLQQRLANLQERLYAESQQALLIVLQARDAGGKDGTVKHVIGAFNPSGVQVSNFKVPTEEERAHDFLWRIHRQTPRLGMIGVFNRSQYEDVLVTRVHHLIDDQTAQRRLKHICAFESLLTDSGTRIVKFYLHISPEEQKKRLEARLADPSKHWKFNPGDLQERAHWDAYTAVYEDVLTTSTPAAPWYVVPADRKWFRNLLVSQILVQTLEEMNPQFPAPAFNAADLRIV

SEQ ID NO: 80 - protein, 295 aa, Mycobacterium tuberculosis
  misc_feature: polyphosphate kinase
MDIPSVDVSTATNDGASSRAKGHRSAAPGRRKISDAVYQAELFRLQTEFVKLQEWARHSGARLVVIFEGRDGAGKGGAIKRITEYLNPRVARIAALPAPTDRERGQWYYQRYIAHLPAKGEIVLFDRSWYNRAGVEKVMGFCTPQEYVLFLRQTPIFEQMLIDDGILLRKYWFSVSDAEQLRRFKARRNDPVRQWKLSPMDLESVYRWEDYSRAKDEMMVHTDTPVSPWYVVESDIKKHARLNMMAHLLSTIDYADVEKPKVKLPPRPLVSGNYRRPPRELSTYVDDYVATLIAR

SEQ ID NO: 81 - protein, 357 aa, Pseudomonas aeruginosa
  misc_feature: polyphosphate kinase
MSEEPTVSPPSPEQPAAQPAKPARPAARRAPRKPATRRPRVASPAQKAREEIQAISQKPVALQVASAPHGSSEDSTSASLPANYPYHTRMRRNEYEKAKHDLQIELLKVQSWVKETGQRVVVLFEGRDAAGKGGTIKRFMEHLNPRGARIVALEKPSSQEQGQWYFQRYIQHLPTAGEMVFFDRSWYNRAGVERVMGFCSPLQYLEFMRQAPELERMLTNSGILLFKYWFSVSREEQLRRFISRRDDPLKHWKLSPIDIKSLDKWDDYTAAKQAMFFHTDTADAPWTVIKSDDKKRARLNCIRHFLHSLDYPDKDRRIAHEPDPLLVGPASRVIEEDEKVYAEAAAAPGHANLDIPA

SEQ ID NO: 82 - protein, 608 aa, Thermus thermophilus
  misc_feature: polyphosphate kinase
MHLLPEASWLQFNRRVLLQTERPDFPLLERLRFLGIWNRNLDEFFAARIAKPFLKSRRGPDHLALLQEALDQAKLARARYQNLLQEAFPRLRVLDPGELDDLDWLYFRVFLAEEVAPKTDLIPWEAAQDLSHSALYFASERYLVRLPQDLPRLVEVPGREGTYVRLGALMRWRSDLLLPEEAPLYEFRVLRLLESERVRADWNELAESLEGRQEGTPTLLVVEEGFPEAWLDALRRALGLFLEEVFALKPPLNLSLVDTLVAQGPPEWRFPPFRPERPRTFLKNPLALLGKRDVLLYHPFEDYAAVERFAEAALAEEVEEVWATLYRTGEENPLAEALIAAARKGKRVHVLLEGRARFDELLNLRWYLRLVRAGVEVLPLPERKVHAKAFLILTREGGYAHLGTGNYNPTNGHHYTDFSLFTARKEVVAEVRAFFQAMAEEKTPRLGLLRTGEGIRRLLLEAVLHEAHPKGRLILKFNHLTDPELLEALVYAASRGARVDLLVRSTLTRLHPAIRAKSLVGRFLEHARAAAFRAGGEWRVYLTSADAMPRNFQNRFELLFPVLDKEAKKKVLKVLKRQVRDDRNSFLLTPEGEKRLWGGRHDAQRLEL

SEQ ID NO: 83 - protein, 475 aa, Acinetobacter johnsonii
  misc_feature: PolyphosphateAMP phosphotransferase
MDTETIASAVLNEEQLSLDLIEAQYALMNTRDQSNAKSLVILVSGIELAGKGEAVKQLREWVDPRFLYVKADPPHLFNLKQPFWQPYTRFVPAEGQIMVWFGNWYGDLLATAMHASKPLDDTLFDEYVSNMRAFEQDLKNNNVDVLKVWFDLSWKSLQKRLDDMDPSEVHWHKLHGLDWRNKKQYDTLQKLRTRFTDDWQIIDGEDEDLRNHNFAQAILTALRHCPEHEKKAALKWQQAPIPDILTQFEVPQAEDANYKSELKKLTKQVADAMRCDDRKVVIAFEGMDAAGKGGAIKRIVKKLDPREYEIHTIAAPEKYELRRPYLWRFWSKLQSDDITIFDRTWYGRVLVERVEGFATEVEWQRAYAEINRFEKNLSSSQTVLIKFWLAIDKDEQAARFKARESTPHKRFKITEEDWRNRDKWDDYLKAAADMFAHTDTSYAPWYIISTNDKQQARIEVLRAILKQLKADRDTD

SEQ ID NO: 84 - protein, 259 aa, Myxococcus xanthus
  misc_feature: PolyphosphateAMP phosphotransferase
MTRKRSQKIDDKTANGYVFPVCPHGTVTLAAVDPSVSAGEHAKYEKTLKTLQERVFELQIQNYLAGRKAVIVFEGWDASGKGGAIRRLTTLMDPRGYKVWPISSPSEEERRHHYLWRFWRKTPGAGEVCMFDRSWYGRVLVERVEGFAKPAEWRRAYDEINAFEHMLTADGVRMVKFFIHIDKKTQLLRFREREADPAKRYKLGPEDWRNRAKWKKYEAAIQEMLDRTHRPDAPWHVVPGNDKRYSRLEVLRRCVELLG

SEQ ID NO: 85 - protein, 217 aa, Clostridium thermocellum
  misc_feature: adenylate kinase
MRLVLLGAPGAGKGTQAVVISQKYNVPHISTGDIFRSNIKNGTELGRKAKEYIDKGLLVPDELTVDIVKDRISQPDCKAGFILDGFPRTIYQAERLDEILKELNVELDCALNIYVPDEEIIKRMSGRRVCSKCGMSYHIVYNQPKVENICDSCNGELIQRDDDKEETVIQRLNTYHKQTEPLIEYYEKKGKLLTVHGQEGVDDTTKEVLNALSGVKL

SEQ ID NO: 86 - protein, 189 aa, Sulfolobus acidocaldarius
  misc_feature: adenylate kinase
MIIIVAGTPGTGKTTVAKALSEKLNLNFLLLSSFIIENKAYTEYDDIRQSYIIDEDKVFELIEKYIENNPNVVIETIYPSLVPKADKIVVLKRDPFILHEELKKRNWNELKIAENLEAEILGVIEAEAIEAFGKDKVCSINNTGRTISQILEKIIKNECEQIDWLDDEKIQNFLLSLDKVISKYEDEKS

SEQ ID NO: 87 - protein, 220 aa, Thermotoga maritima
  misc_feature: adenylate kinase
MMAYLVFLGPPGAGKGTYAKRLQEITGIPHISTGDIFRDIVKKENDELGKKIKEIMERGELVPDELVNEVVKRRLSEKDCERGFILDGYPRTVAQAEFLDGFLKTQNKELTAAVLFEVPEEVVVQRLTARRICPKCGRIYNLISLPPKEDELCDDCKVKLVQREDDKEETVRHRYKVYLEKTQPVIDYYDKKGILKRVDGTIGIDNVIAEVLKIIGWSDK

SEQ ID NO: 88 - protein, 168 aa, Methanocaldococcus jannaschii
  misc_feature: nicotinamide nucleotide adenylyltransferase (NMNAT)
MRGFIIGRFQPFHKGHLEVIKKIAEEVDEIIIGIGSAQKSHTLENPFTAGERILMITQSLKDYDLTYYPIPIKDIEFNSIWVSYVESLTPPFDIVYSGNPLVRVLFEERGYEVKRPEMFNRKEYSGTEIRRRMLNGEKWEHLVPKAVVDVIKEIKGVERLRKLAQTDK

SEQ ID NO: 89 - protein, 401 aa, Saccharomyces cerevisiae
  misc_feature: nicotinamide nucleotide adenylyltransferase (NMNAT)
MDPTRAPDFKPPSADEELIPPPDPESKIPKSIPIIPYVLADANSSIDAPFNIKRKKKHPKHHHHHHHSRKEGNDKKHQHIPLNQDDFQPLSAEVSSEDDDADFRSKERYGSDSTTESETRGVQKYQIADLEEVPHGIVRQARTLEDYEFPSHRLSKKLLDPNKLPLVIVACGSFSPITYLHLRMFEMALDAISEQTRFEVIGGYYSPVSDNYQKQGLAPSYHRVRMCELACERTSSWLMVDAWESLQPSYTRTAKVLDHFNHEINIKRGGVATVTGEKIGVKIMLLAGGDLIESMGEPNVWADADLHHILGNYGCLIVERTGSDVRSFLLSHDIMYEHRRNILIIKQLIYNDISSTKVRLFIRRAMSVQYLLPNSVIRYIQEHRLYVDQTEPVKQVLGNKE

SEQ ID NO: 90 - protein, 307 aa, Leishmania braziliensis
  misc_feature: nicotinamide nucleotide adenylyltransferase (NMNAT)
MLSSTAAPYALRTDKLKPLEGCAASCPTSTAEAASQVTPLLQPVVLVICGSFNPIHNAHLKLYDAAKRSIEGVDGRVVLGGFLSPVGDAYGKPGLRCAADRVQVMEKALCHHPELNVDTWECQQPTYTRTFFVLRALEEHVNAWYAQSEPAAMEWLTSHGRHVRVVFACGADLFFSFWRPGCWSLCLLRQLLDSFPLVVVQRGGARGNISDSDDFARVCQTAPLLWETAEDGERIEIDMLRYTFTFAAFSVPDDTSSTAVRNAVMELAKMPVDEVTARTALQSRLRTMLPEAAIQLVVDLYGEGLPS

SEQ ID NO: 91 - protein, 340 aa, Clostridium thermocellum
  misc_feature: nicotinamide nucleotide adenylyltransferase (NMNAT)
MYNVGIYGGSFNPLHLGHIRCIIEAANQCKELHIIISCGVNRNEIPPRVRYRWIYQVTKHIGNVKIHFLEDDAVDKNAYSKEYWQEDAQKVKDMVGKPIDVVFCGSDYDENSFWKQCYPESELYIIKRNGISSTEIRKNPYAHWDSIPNVVREYYVKKVLLIGGESTGKSTLTINLANYYNTNYVEEVGREISMRSGTDMLMIPSDFTDILLTHKMKELEAAKQSNKVLFIDTDCLITRFYIDFLDDPQNDKERNKALADAISALNHYDLVFYLEPDVEFVQDGDRSEVIAANREKYGNQIKKLFDERGIKYISVSGNYHERFLRVTSEVDRMLGINRAE

SEQ ID NO: 92 - protein, 410 aa, Escherichia coli (strain K12)
  misc_feature: nicotinamide nucleotide adenylyltransferase (NMNAT)
MSSFDYLKTAIKQQGCTLQQVADASGMTKGYLSQLLNAKIKSPSAQKLEALHRFLGLEFPRQKKTIGVVFGKFYPLHTGHIYLIQRACSQVDELHIIMGFDDTRDRALFEDSAMSQQPTVPDRLRWLLQTFKYQKNIRIHAFNEEGMEPYPHGWDVWSNGIKKFMAEKGIQPDLIYTSEEADAPQYMEHLGIETVLVDPKRTFMSISGAQIRENPFRYWEYIPTEVKPFFVRTVAILGGESSGKSTLVNKLANIFNTTSAWEYGRDYVFSHLGGDEIALQYSDYDKIALGHAQYIDFAVKYANKVAFIDTDFVTTQAFCKKYEGREHPFVQALIDEYRFDLVILLENNTPWVADGLRSLGSSVDRKEFQNLLVEMLEENNIEFVRVEEEDYDSRFLRCVELVREMMGEQR

SEQ ID NO: 93 - protein, 410 aa, Salmonella typhimurium
  misc_feature: nicotinamide nucleotide adenylyltransferase (NMNAT)
MSSFDYLKTAIKQQGCTLQQVADASGMTKGYLSQLLNAKIKSPSAQKLEALHRFLGLEFPRRQKNIGVVFGKFYPLHTGHIYLIQRACSQVDELHIIMGYDDTRDRGLFEDSAMSQQPTVSDRLRWLLQTFKYQKNIRIHAFNEEGMEPYPHGWDVWSNGIKAFMAEKGIQPSWIYTSEEADAPQYLEHLGIETVLVDPERTFMNISGAQIRENPFRYWEYIPTEVKPFFVRTVAILGGESSGKSTLVNKLANIFNTTSAWEYGRDYVFSHLGGDEMALQYSDYDKIALGHAQYIDFAVKYANKVAFIDTDFVTTQAFCKKYEGREHPFVQALIDEYRFDLVILLENNTPWVADGLRSLGSSVDRKAFQNLLVEMLKENNIEFVHVKEADYDGRFLRCVELVKEMMGEQG

SEQ ID NO: 94 - protein, 220 aa, Thermotoga maritima
  misc_feature: 6-phosphogluconolactonage
MEKTVIYLLEDGYVDFVVEKIRTKMEKLLEEKDKIFVVLAGGRTPLPVYEKLAEQKFPWNRIHFFLSDERYVPLDSDQSNFRNINEVLFSRAKIPSGNVHYVDTSLPIEKACEKYEREIRSATDQFDLAILGMGPDGHVASIFDLETGNKDNLVTFTDPSGDPKVPRVTLTFRALNTSLYVLFLIRGKEKINRLTEILKDTPLPAYFVRGKEKTVWFVGK

SEQ ID NO: 95 - protein, 327 aa, Trypanosoma brucei brucei
  misc_feature: adenosine nucleosidase, purine nucleosidase
MAKTVILDHDGNKDDFVAMILLLSNPKKVNLIGCICTDADCFVENGFDVTGKIMCAMHRLIKTPLFPIGKSTATAVNAFPTEWRFSAKNLDDMPFLNIVEDVALWEKLKPENEAHNGQQLLADLVMKSKEKVTVCVTGPLSNMAWCIEKYGEAFTSKVEECVIMGGAVDVGGNVFLPTTDGSAEWNIYWDPPAAKKVLCCPNIRCVLFSLDATNTVPVRSVDVKGFGAQNQYLLSQMVGTMWAMSTHEEILRDGDAYYAWDALTAAYILEPTIATLEPVALDVDVSKGKSEGRTPRASGEGKPCVHVARNPSKQMFHDLVFASTRVC

SEQ ID NO: 96 - protein, 321 aa, Bacillus thuringiensis subsp. kurstaki
  misc_feature: adenosine nucleosidase, purine nucleosidase
MRIVNKKIIFFGDFGIDDAVALIYANKTCKLDILGIVAEYGNVSRDIVTENVYFLERYYATQVKIIEGASRPMTAEEPLFFPEIHGDHGLGPIIPPEMRICKRENFCELIKLIEPCPEDIIIVATGRLTTLATLFLLYPNIMDRICSYYIMGGAFLFPGNVTPVSEANFYGDPIAVNIVMKYAKNASIYPLNVTQGALITPEMANIIDKQGTGQAKLIKPMIDFYYENFYKKEYPGIGGSPIHDLLPFISFINDSIFEYKKSAVWISTTNDVTRGQSVADFRKIAEPTRFDDRPIQRIAVGFNYAAFKEEFMRTILKPDCP

SEQ ID NO: 97 - protein, 304 aa, Escherichia coli (strain K12)
  misc_feature: adenosine nucleosidase, purine nucleosidase
MRLPIFLDTDPGIDDAVAIAAAIFAPELDLQLMTTVAGNVSVEKTTRNALQLLHFWNAEIPLAQGAAVPLVRAPRDAASVHGESGMAGYDFVEHNRKPLGIPAFLAIRDALMRAPEPVTLVAIGPLTNIALLLSQCPECKPYIRRLVIMGGSAGRGNCTPNAEFNIAADPEAAACVFRSGIEIVMCGLDVTNQAILTPDYLSTLPQLNRTGKMLHALFSHYRSGSMQSGLRMHDLCAIAWLVRPDLFTLKPCFVAVETQGEFTSGTTVVDIDGCLGKPANVQVALDLDVKGFQQWVAEVLALAS

SEQ ID NO: 98 - protein, 454 aa, Escherichia coli (strain K12)
  misc_feature: pyrimidine/purine nucleotide monophosphate nucleosidase (PPMPN)
MITHISPLGSMDMLSQLEVDMLKRTASSDLYQLFRNCSLAVLNSGSLTDNSKELLSRFENFDINVLRRERGVKLELINPPEEAFVDGRIIRALQANLFAVLRDILFVYGQIHNTVRFPNLNLDNSVHITNLVFSILRNARALHVGEAPNMVVCWGGHSINENEYLYARRVGNQLGLRELNICTGCGPGAMEAPMKGAAVGHAQQRYKDSRFIGMTEPSIIAAEPPNPLVNELIIMPDIEKRLEAFVRIAHGIIIFPGGVGTAEELLYLLGILMNPANKDQVLPLILTGPKESADYFRVLDEFVVHTLGENARRHYRIIIDDAAEVARQMKKSMPLVKENRRDTGDAYSFNWSMRIAPDLQMPFEPSHENMANLKLYPDQPVEVLAADLRRAFSGIVAGNVKEVGIRAIEEFGPYKINGDKEIMRRMDDLLQGFVAQHRMKLPGSAYIPCYEICT

SEQ ID NO: 99 - protein, 454 aa, Shigella flexneri; identical to SEQ ID NO: 98
  misc_feature: pyrimidine/purine nucleotide monophosphate nucleosidase (PPMPN)

SEQ ID NO: 100 - protein, 485 aa, Mannheimia succiniciproducens
  misc_feature: pyrimidine/purine nucleotide monophosphate nucleosidase (PPMPN)
MADWILILSVLILRRFRKIYAWQDNKNIGDIMSTSHYVSPKGSMDQLSHMEIDLLTKRAQSDLYKLFRNSSLAVLNSGAINDDSRALLNKYPNFEISIICKERGVTLKLDNSPESAFVDDKIIRNIQYNLFAVLRDILFVNALMQRFGLDAERGNSFITNQVFSILRNAKALSLNEDPNLVVCWGGHSINQTEYAYCRAVGLELGLRELNIVTGCGPGVMEAPMKGAAIGHANQRYKQSRFIGITEPSIIASEPPNPIVNELIIMPDIEKRLEAFVRMGHGIVIFPGGPGTFEEFMFILGIKLNPENRAQKLPLILTGPKESADYFATIDRFVLDTLGEEAQSLYTIIIDDAVAVARHMKAEMVEIRDFRCKISDSFSFNWSLKIEHQFQQPFLPTHENMANLNLHLNQSTVDLAANLRCVFSGIVAGNIKPATQDQIAEKGKFQLYGEPRLMEKVDNLLQDFIVQHRMKLPTDEAYEPCYEICK

SEQ ID NO: 101 - protein, 156 aa, Bacillus subtilis (strain 168)
  misc_feature: guanine deaminase
MNHETFLKRAVTLACEGVNAGIGGPFGAVIVKDGAIIAEGQNNVTTSNDPTAHAEVTAIRKACKVLGAYQLDDCILYTSCEPCPMCLGAIYWARPKAVFYAAEHTDAAEAGFDDSFIYKEIDKPAEERTIPFYQVTLTEHLSPFQAWRNFANKKEY

SEQ ID NO: 102 - protein, 337 aa, Haloferax volcanii
  misc_feature: guanine deaminase
MQLEGTILRGRDFDPVEGRVVVEEGTITAIEETATESTDIILPAFVNAHTHLGDSIAKEAGEGLSLEDLVAPPDGLKHRLLRAASTGEKAAAMHRSLRMMEAGGTAACLEFREGGVEGVDVIRRALDGRDIEAVVFGRETADAMEIADGFGASGARDGEFGALRNATEAAGKLFGIHAGERDAHDINAALDLDPDFLVHMVHPEALHLERVEDSEIPIVICPRSNLVTDAGVAPIRELVDRTTVALGTDNVMLNSPSMFREMEFTSKLADVSAAEVLRMATVNGADIAGLNYGLVEEGRDAKLLVLDGDTDNLAGVEDVVRAVVRRAGQADVKDVYL

SEQ ID NO: 103 - protein, 437 aa, Haloferax volcanii
  misc_feature: guanine deaminase
MLLAGTVVVDASTIIEEGAVVVDGDRIVAVGAHADLTDRYPNHERREFGIVAPGLVGGHVHSVQSLGRGIADDTELLDWLFDHVLPMEAGLDAEGMRVAAELGYLECIESGTTTVVDHLSVRHAEEAFEAAGEMGIRGRIGKVLMDTNAPEGLQEDTDAGLAESERLIERYHDSFDGRIQYAVTPRFAVTCSEACLRGARDLADRYDGVRIHTHASENRSEIETVEDETGMRNIHWLDEVGLTGDDVVLAHCVHTDDSERDVLAETGTHVTYCPSSNMKLASGIAPIPDYLDRGINVALGNDGPPCNNTLDPFTEMRQASLLQKVDALDPTSTPAATVFEMATRNGAKAAGFDRVGELREGWKADIVGIDADRTRATPLHDVLSHLVFAAHGDDVVFTMVDGDVLYDEGEHVRADADGIRKRARAVADGLDSAPTSD

SEQ ID NO: 104 - protein, 134 aa, Halobacterium salinarum
  misc_feature: guanine deaminase
MDTDDLLAAARDATAAAHVPYSEYKVGAALDTASGAVYTGCNIENANYSNSLHAEEVAVGKAVSAGDTDFQRLAVTSSQRDAVTPCGMCRQTLAEFAADDLTILCDSGDDTAGVVTYTLGELLPDTITASNLDA

SEQ ID NO: 105 - protein, 179 aa, Thermoplasma acidophilum
  misc_feature: diphosphatase (DPP)
MESFYHSVPVGPKPPEEVYVIVEIPRGSRVKYEIAKDFPGMLVDRVLYSSVVYPVDYGLIPRTLYYDGDPMDVMVLISQPTFPGAIMKVRPIGMMKMVDQGETDNKILAVFDKDPNVSYIKDLKDVNAHLLDEIANFFSTYKILEKKETKVLGWEGKEAALKEIEVSIKMYEEKYGKKN

SEQ ID NO: 106 - protein, 178 aa, Pyrococcus furiosus
  misc_feature: diphosphatase (DPP)
MNPFHDLEPGPDVPEVVYAIIEIPKGSRNKYELDKKTGLLKLDRVLYSPFFYPVDYGIIPRTWYEDDDPFDIMVIMREPVYPLTIIEARPIGLFKMIDSGDKDYKVLAVPVEDPYFKDWKDIDDVPKAFLDEIAHFFKRYKELQGKEIIVEGWEGAEAAKREILRAIEMYKEKFGKKE

SEQ ID NO: 107 - protein, 220 aa, Clostridium thermocellum
  misc_feature: D-xylulose 5-phosphate 3-epimerase (RuPE)
MLIKIAPSILSADFSKLGEEVLRIEKAGADLVHIDVMDGHFVPNITIGPPVVKSLKKVSKLPFDVHLMIENPDMYIESFADAGADIISVHAEACRHLHRTIQKIKQLGIKAAVALNPATPLCAVEWVMEDVDMILLMTVNPGFGGQKYINGVTRKIEELERIVESRGLNIDIEVDGGIDTETVQYVTKAGANVIVSGSTIFAAEDTAKIIQELRMNSYRK

SEQ ID NO: 108 - protein, 220 aa, Thermotoga maritima
  misc_feature: D-xylulose 5-phosphate 3-epimerase (RuPE)
MVKIAASILACDLARLADEVKRVEEHVDMIHFDVMDGHFVPNISFGLPVLKALRKETKLPISVHLMITNPEDYIDRFIEEGADMVAIHYESTFHLHRLVHRVKDLGARAFVAINPHTPINLLSEIITDVDGVLVMSVNPGFSGQRFIARSLEKIRNLRKMVKELGLETEIMVDGGVNEENASILVKNGATILVMGYGIFRNDNYVELIKSIKQEREEFAD

SEQ ID NO: 109 - protein, 224 aa, Pseudomonas cichorii JBC1
  misc_feature: pentose 3-epimerase
MQPFVIAPSILSADFARLGEDVDSVLAAGADFVHFDVMDNHYVPNLTIGPMVCSALRKYGVTAPIDVHLMVSPVDRIIGDFIEAGATYITFHPEATQHIDRSLQLIREGGCKAGLVFNPATPLNLLEYVMDKVDMILLMSVNPGFGGQKFIPGTLNKLREARALIDASGRDIRLEIDGGVNVANIREIAAAGADTFVAGSAIFNAPDYKEVIEKMRNELASARA

SEQ ID NO: 110 - protein, 295 aa, Rhodobacter sphaeroides
  misc_feature: pentose 3-epimerase
MKNPVGIISMQFIRPFTSESLHFLKKSRALGFDFIELLVPEPEDGLDAAEVRRICEGEGLGLVLAARVNLQRSIASEEPAARAGGRDYLKYCIEAAEALGATIVGGPLYGEPLVFAGRPPFPWTAEQIAARAARTVEGLAEVAPLAASAGKVFGLEPLNRFETDIVNTTAQAIEVVDAVGSPGLGVMLDTFHMNMEERSIPDAIRATGARLVHFQANENHRGFPGTGTMDWTAIARALGQAGYAGPVSLEPFRRDDERVALPIAHWRAPHEDEDEKLRAGLGLIRSALTLAEVTH

SEQ ID NO: 111 - protein, 387 aa, Thermus thermophilus
  misc_feature: D-xylose isomerase (D-XI)
MYEPKPEHRFTFGLWTVGNVGRDPFGDAVRERLDPVYVVHKLAELGAYGVNLHDEDLIPRGTPPQERDQIVRRFKKALDETGLKVPMVTANLFSDPAFKDGAFTSPDPWVRAYALRKSLETMDLGAELGAEIYVVWPGREGAEVEATGKARKVWDWVREALNFMAAYAEDQGYGYRFALEPKPNEPRGDIYFATVGSMLAFIHTLDRPERFGLNPEFAHETMAGLNFVHAVAQALDAGKLFHIDLNDQRMSRFDQDLRFGSENLKAAFFLVDLLESSGYQGPRHFDAHALRTEDEEGVWAFARGCMRTYLILKERAEAFREDPEVKELLAAYYQEDPAALALLGPYSREKAEALKRAELPLEAKRRRGYALERLDQLAVEYLLGVRG

SEQ ID NO: 112 - protein, 307 aa, Thermotoga neapolitana
  misc_feature: D-xylose isomerase (D-XI)
MQGMVKGIGTNVDTRRVNGSIKRLVSELEFFKSIGFDYVEIPAAGLDVIARGRIIKKRLERVKEILSNYNFRYTVHAPDVINLKRKTNPWHYKVMEAVIEFAGEINAEVIVYHYNEVDHSIDVPERIQRRAEILALRDLADLAKEKGIVIGIENVQHPVSEVLELVKQVNHPNVKLTIDIGHLFILTSYRGLNFYEELKKGLPYAVELHVSDNFGESPQTYQQIPDVEAFRFVYGIGDLHLPIGEGDIPYNRVLKIIRESDFSGIVILEINSMDRFADEYADSLNLLRRRLILAANNSKTKKRRRVK

SEQ ID NO: 113 - protein, 441 aa, Geobacillus stearothermophilus
  misc_feature: D-xylose isomerase (D-XI)
MPYFDNISTIAYEGPASKNPLAFKFYNPEEKVGDKTMEEHLRFSVAYWHTFTGDGSDPFGAGNMIRPWNKYSGMDLAKARVEAAFEFFEKLNIPFFCFHDVDIAPEGETLKETYKNLDIIVDMIEEYMKTSKTKLLWNTANLFTHPRFVHGAATSCNADVFAYAAAKVKKGLEIAKRLGAENYVFWGGREGYETLLNTDMKLELDNLARFLHMAVDYAKEIGFDGQFLIEPKPKEPTKHQYDFDVATALAFLQTYGLKDYFKFNIEANHATLAGHTFEHELRVARIHGMLGSVDANQGDMLLGWDTDEFPTDLYSTTLAMYEILKNGGLGRGGLNFDAKVRRGSFEPEDLFYAHIAGMDSFAVGLKVAHRLIEDRVFDEFIEERYKSYTEGIGREIVEGTADFHKLEAHALQLGEIQNQSGRQERLKTLLNQYLLEVCAAR

SEQ ID NO: 114 - protein, 149 aa, Clostridium thermocellum
  misc_feature: ribose 5-phosphate isomerase (RPI)
MKIGIGSDHGGYNLKREIADFLKKRGYEVIDFGTHGNESVDYPDFGLKVAEAVKSGECDRGIVICGTGLGISIAANKVPGIRAAVCTNSYMARMSREHNDANILALGERVVGLDLALDIVDTWLKAEFQGGRHATRVGKIGEIEKKYSK

SEQ ID NO: 115 - protein, 143 aa, Thermotoga maritima
  misc_feature: ribose 5-phosphate isomerase (RPI)
MKIAIASDHAAFELKEKVKNYLLGKGIEVEDHGTYSEESVDYPDYAKKVVQSILSNEADFGILLCGTGLGMSIAANRYRGIRAALCLFPDMARLARSHNNANILVLPGRLIGAELAFWIVDTFLSTPFDGGRHERRIRKIDEV

SEQ ID NO: 116 - protein, 320 aa, Geobacillus thermodenitrificans
  misc_feature: mannose 6-phosphate isomerase
MHQEPIFLTPVFQERIWGGTKLAERFGYDIPSSQTGECWAVSAHPHGQTVVARGPFQGMTLGQLWEERRDLFGNFPSDRFPLLTKILDANADLSVQVHPDDDYAKTNEGGELGKTECWYIIDCKPGAQLIYGHYAQTKEELRAMMEAGEWDRLLRKVPIHPGDFFYVPSGTIHALCEGTLVLETQQSSDTTYRVYDYDRVDSQGRKRELHLEKAIDVTTVPHRDTDVQPHVANIPGATVTTFVEGDYFGVQKWHVHGEAEWEQTKPFLIVSILQGEGELVHGERTYPIRQGDHFILPHQFGRFAIRGTLEAIASWPRTSK

SEQ ID NO: 117 - protein, 387 aa, Mycolicibacterium smegmatis
  misc_feature: D-ribose isomerase
MSAINVHPALDNLAIELPSWAFGNSGTRFKVFGTPGTPRTVEEKIADAAMVHRMTGLAPRVALHIPWDTVDDYAALGVYAREQGVELGTINSNTFQDDDYKFGSLTHTDKGVRQKAIDHHLACIEIMNQTGSRDLKIWLADGTNYPGQGDIRGRQDRLAESLAAIYQHIGPDQRMVLEYKFFEPAMYMTDVPDWGTAYAQVSALGERAVVCLDTGHHAPGTNIEFIVAQLLRLGKLGSFDFNSRFYADDDLIVGAADPFQLFRILFEVIRGGGLDPSSGVALMLDQCHNVEAKIPGQIRSVLNVAEMTARALLVDTEALAEAQENGDVLGANGIFMDAFYTDVRPALAKWRVDRGLPGDPMAAYAASGYQDTINAERVGGTQSSWGA

SEQ ID NO: 118 - protein, 578 aa, Clostridium thermocellum
  misc_feature: phosphoglucomutase
MRSSALYKFWVENDYFDAETKKELLSIKDNPKEIEERFYKDLEFGTGGLRGIIGAGTNRINIYTVRKASQGLADYIKSLGLQDRGIAIAYDSRYKSPEFALEAAKVFAGNGIKAFLFDELRPTPELSFTVRHLNAAAGVVITASHNPKEYNGYKVYGEDGGQLPVEASNKVISYINKIEDITQVKVMEKDEAIEKGLLRIIGKEIDDEYISKLKTLSANPELAAEIGKTFKIVYTPLHGAGNKPVRRILDEIGFKNVLVVKEQELPDSEFSTVKSPNPEEREAFELAIELAKKENVDLIIGTDPDCDRVGIVVRNKEGEYVPLTGNQTGCLLLEYILSQKKQRGELPENGFVVKTIVTTELARAITDAYNVELVEVLTGFKFIGEKIKQLDEFGDKKYLFGFEESYGYLAGTFARDKDAVVASMLIAEMAAYYKSRGLTLYEGLMELLEKYGYTLEGITSFTLKGKDGVEKIKSAMKNLRENRVVKFGEYEAVAVRDYLTSERYEVATGAKEKLTLVESDVLYYELKDKAWFCIRPSGTEPKIKIYYGVTEKSMDAAKEKLKHLQDNVLSVIEPLLKD

SEQ ID NO: 119 - protein, 524 aa, Thermus thermophilus
  misc_feature: phosphoglucomutase
MEITRLLTLYYEATPDPQNPLEGVRFGTSGHRGSSLKATFTEAHVLAIAQAIAELRPSFGATGPLFLAKDTHALSEPAWATALSVFAAHGIEVRVEADGDYTPTPLVSLAILEHNARQEAKADGVLLTPSHNPPEDGGFKYNPPTGGPANARITRAIEERANALLQEGLKGVKRLPLREALARAKPFDYAGLYVEKVAEAVDLEAIRASGLRIGVDPLGGASLRVWERLAESHGLPLEVVNPTLDPTFRFMPKDHDGKIRMDCSSPYAMAGLLALKDRFDLAIGNDPDADRHGIVTPRGLMNPNHYLAAALHHLYTTRSWPGAKVGKTAVTSALLDRVAQALGREVYETPVGFKHFVAGLLEGWLGFAGEESAGASFLRFDGRPFSTDKDGILMGLLAAELMAKRGQAPDALYEALAETLGRPYYARKDLPVSPEAKARLARLSTKEVHPATLAGEPVLQVLDRATGNGEPLGGIKVVAANAWFAVRPSGTEDVAKVYAESFLGEAHLERVLEEATALLHKALA

SEQ ID NO: 120 - protein, 390 aa, Thermotoga maritima
  misc_feature: phosphopentomutase
MRVVLIVLDSVGIGEMPDAHLYGDEGSNTIVNTAKAVSGLHLPNMAKLGLGNLDDIPGVEPVKPAEGIYGKMMEKSPGKDTTTGHWEIAGVILKKPFDLFPEGFPKELIEEFERRTGRKVIGNKPASGTEIIKELGPIHEKTGALIVYTSADSVFQIAAKKEIVPLEELYRYCEIARELLNEMGYKVARVIARPFTGEWPNYVRTPERKDFSLEPEGKTLLDVLTENGIPVYGVGKIADIFAGRGVTENYKTKDNNDGIDKTISLMKEKNHDCLIFTNLVDFDTKYGHRNDPVSYAKALEEFDARLPEIMHNLNEDDVLFITADHGCDPTTPSTDHSREMVPLLGYGGRLKKDVYVGIRETFADLGQTIADIFGVPPLENGTSFKNLIWE

SEQ ID NO: 121 - protein, 381 aa, Thermus thermophilus
  misc_feature: phosphopentomutase
MKAVAIVLDSVGLGYLPDAPLFGDEGADTLDHTVLKTGIALPHLAGLGLGRVPGVHTLPRAERPRGGFGRMREVNPGKDTTTGHWEFVGIHLEKPFRTFPQGFPEDVLREWAEAIGVGGWLLNRPYSGTEAIRDYGEAHLKTGYPIVYTSADSVFQVAAHVDVVPVEELYRFCQVARERLVGELQVARVIARPFAGEPGRFYRLEHLRKDFALEPPRNVLDVLREGGLEVVGVGKIPDIYAGRGFTRKVKTKDNQDGLEKTLALMGEPFSGLVFTNLVDFDSKYGHRRDPEGYGRALVELDAFLPRLLAALGPEDHLFLVSDHGNDPTFFGTDHTREYAMLLWVGPGVEGELGTRETFADLGATWARLFGLAWDGPGTSLV

SEQ ID NO: 122 - protein, 388 aa, Clostridium thermocellum
  misc_feature: phosphopentomutase
MKRAIIIVLDSVGMGELPDAAKYGDEGSNTLGNIAKNLPDFSLPNLESLGLGNIDGMTGYEPSKNPLGSYGRMAEKSAGKDTTTGHWEIAGLILDKPFPVYPNGFPEDIIKRFEDSIGTKTLGNVPASGTEIIKLLGDEHVKTGYPIVYTSADSVFQIAAHENVIPVERLYDMCRTARNILTGEHAVGRVIARPFIGESGNYKRTDRRKDFSLAPVGKTLLDYAVENGYKVKAVGKIEDIFGGRGITESVHIHDNMDGVDRTLEYMRDDFEGILFTNLVDFDMLYGHRNDIAGYANALKEFDRRIPEILANLREDDLLVITADHGCDPSTESTDHSREYVPLLVYGKKFKSNVNLGTRSTFADVAKTVAHYLGISSNLEGESFLGSIL

(19) United States
(12) Patent Application Publication
(10) Pub. No.: US 2021/0246476 A1
(43) Pub. Date: Aug. 12, 2021

(54) BIOSYNTHESIS OF PREPARING NICOTINAMIDE MONONUCLEOTIDE AND DERIVATIVES THEREOF

(71) Applicant: Yi Heng Percival ZHANG, Blacksburg, VA (US)

(72) Inventor: Yi Heng Percival ZHANG, Blacksburg, VA (US)

(21) Appl. No.: 17/164,010

(22) Filed: Feb. 1, 2021

Related U.S. Application Data
(60) Provisional application No. 62/968,606, filed on Jan. 31, 2020.

(51) Int. Cl. C12P 19/30

(52) U.S. Cl. CPC C12P 19/30; C12Y 204/02001; C12Y 204/02012; C12Y 204/02008; C12Y 207/01022

(57) ABSTRACT

A method of making nicotinamide mononucleotide (NMN), nicotinamide mononucleotide derivatives, or mixtures thereof is disclosed. The method involves the in vitro artificial enzymatic pathways comprised: the generation of alpha-D-ribose-1-phosphate from numerous substrates followed by the synthesis of nicotinamide mononucleotide catalyzed by nicotinamide riboside phosphorylase and nicotinamide riboside kinase or the generation of 5-phospho-alpha-D-ribose-1-diphosphate from nucleotides followed by the synthesis of nicotinamide mononucleotide catalyzed by nicotinamide phosphoribosyltransferase. The multiple enzymes were reconstituted in one pot, wherein in-situ removal of byproducts that can be converted to other non-inhibitory chemicals with supplementary enzymes push the overall biotransformation toward the synthesis of nicotinamide mononucleotide. Furthermore, nicotinamide mononucleotide can be converted to its derivatives—nicotinamide adenine dinucleotide and nicotinamide adenine dinucleotide phosphate.

### INCORPORATION OF SEQUENCE LISTING

This application contains a sequence listing in Computer Readable Form (CRF). The CFR file containing the sequence listing entitled “PA572-0001_ST25.txt”, which was created on Feb. 1, 2021, and is 437,797 bytes in size. The information in the sequence listing is incorporated herein by reference in entirety.

### FIELD OF THE INVENTION

The present disclosure is directed generally to methods for the biosynthesis of nicotinamide mononucleotide (NMN), nicotinamide mononucleotide derivatives, or mixtures thereof. More specifically, the present disclosure is directed to enzymes, multiple-enzyme pathways, and methods to adjust reaction conditions and components in order to maximize the conversion of low-cost starting materials towards desired products in high yields.

### BACKGROUND OF THE INVENTION

Nicotinamide mononucleotide (NMN) is a phosphate ester nucleotide derived from nicotinamide riboside (NR). NMN exists in two forms: an oxidized and reduced form, abbreviated as NMN⁺ and NMNH, respectively. Humans can transport NMN across the cell membrane and then generate NAD, which promotes cellular NAD production and counteract age-associated pathologies associated with a decline in tissue NAD levels. Supplementation of NMN increases arterial SIRT1 activity and reverses age-associated arterial dysfunction and oxidative stress. NMN supplementation may represent a novel therapy to restore SIRT1 activity and reverse age-related arterial dysfunction by decreasing oxidative stress (de Picciotto et al. (2016). “Nicotinamide mononucleotide supplementation reverses vascular dysfunction and oxidative stress with aging in mice.” Aging cell 15(3): 522-530). Long-term uptake of NMN mitigates age-associated physiological decline in mice. Orally administered NMN was quickly utilized to synthesize NAD in tissues. NMN effectively mitigates age-associated physiological decline in mice. Without any obvious toxicity or deleterious effects, NMN suppressed age-associated body weight gain, enhanced energy metabolism, promoted physical activity, improved insulin sensitivity and plasma lipid profile, and ameliorated eye function and other pathophysiologies (Mills et al. (2016). “Long-Term Administration of Nicotinamide Mononucleotide Mitigates Age-Associated Physiological Decline in Mice.” Cell Metabolism. 24(6): 795-806).

Nicotinamide adenine dinucleotide (NAD) is a coenzyme that is central to metabolism in all living cells, carrying electrons from one biochemical to another. It consists of two nucleotides (i.e., adenine and nicotinamide) joined through their phosphate groups. NAD exists in two forms: an oxidized and reduced form, abbreviated as NAD⁺ and NADH, respectively. Nicotinamide adenine dinucleotide phosphate (NADP) is a coenzyme used in anabolic reactions. NADP exists in two forms: an oxidized and reduced form, abbreviated as NADP⁺ and NADPH, respectively.

Niacin or nicotinic acid (NA) is a simplest form of vitamin B3, an essential human nutrient. Nicotinamide (NAM) is another form of vitamin B3 found in food and used as a dietary supplement and medication. NAM is better than NA. NAM is added into grains (such as rice and wheat flour) in numerous countries, such as USA for the prevention of pellagra (niacin deficiency). Nicotinamide riboside (NR) is be another new form of vitamin B3, a compound that NAM is linked with D-ribose. Their structures and names are present in FIG. 1.

Nicotinamide riboside phosphorylase (NRP) can irreversibly synthesize NR from NAM and R1P (Reaction 1).

nicotinamide (NAM)+R1P→nicotinamide riboside (NR)+Pᵢ [1]

where → denotes a reversible reaction, Pᵢ is an inorganic orthophosphate anion. NRP is a kind of purine nucleoside phosphorylase (PNP, EC 2.4.2.1), some of which have a promiscuous NRP activity using nicotinamide although nicotinamide is not a purine. For example, several PNPs from Homo sapiens (human) (Grossman, L. and N. O. Kaplan (1958). “NICOTINAMIDE RIBOSIDE PHOSPHORYLASE FROM HUMAN ERYTHROCYTES: II. NICOTINAMIDE SENSITIVITY.” Journal of Biological Chemistry 231(2): 727-740), Bos taurus (Imai, T. and B. M. Anderson (1987). “Nicotinamide riboside phosphorylase from beef liver: Purification and characterization.” Archives of Biochemistry and Biophysics 254(1): 253-262), Escherdia coli (Wielgus-Kutrowska, B., E. Kulikowska, J. Wierzchowski, A. Bzowska and D. Shugar (1997). “Nicotinamide riboside, an unusual, non-typical, substrate of purified purine-nucleoside phosphorylases.” European Journal of Biochemistry. 243(1-2): 408-414), and Cellulomonas sp. (Velasquez J E, Green P R, Wos J A (2015) Method for preparing nicotinamide riboside. US20170121746A1), have validated NPR activities in vitro.

Nicotinamide riboside kinase (NRK, EC 2.7.1.22) can synthesize NMN from NR and adenosine triphosphate (ATP) (Reaction 2), which can be regenerated by numerous well-known means including an enzymatic ATP regeneration system or ATP-generating permeabilized living microorganisms (e.g., Escherichia coli, Saccharomyces cerevisiae) that continuously make ATP (Chen, H.-G., and Zhang, Y.-H. P. J. (2020) “Enzymatic Regeneration and Conservation of ATP: Challenges and Opportunities” Critical Review in Biotechnology, Epub, doi.org/10.1080/07388551.07382020.01826403).

nicotinamide riboside (NR)+ATP→nicotinamide mononucleotide (NMN)+ADP [2]

where → denotes an irreversible reaction with a K_{eq} (equilibrium constant) value of more than 100.

Hypoxanthine/guanine phosphoribosyltransferase (HGPRT, EC 2.4.2.8) can reversibly synthesize 5-phospho-alpha-D-ribose-1-diphosphate (PRPP) from nucleotides, such as inosine monophosphate (IMP) or guanosine monophosphate (GMP) or adenosine monophosphate (AMP), and diphosphate (PPᵢ), cogenerating hypoxanthine or guanosine or adenosine, respectively. Reaction 3 shows a representative biochemical reaction starting with IMP.

inosine monophosphate (IMP)+diphosphate (PPᵢ) [symbol] hypoxanthine+PRPP [3]

Nicotinamide phosphoribosyltransferase (NAMPT, EC 2.4.2.12) can reversibly convert PRPP and NAM to NMN and PPᵢ (Reaction 4).

PRPP+NAM [symbol] NMN+PPᵢ [4]

NR can be synthesized by using organic chemical methods (Sauve, A. A. and T. Yang (2006). Nicotinoyl riboside compositions and methods of use. WO2007061798A2; Felczak K Z (2017) Synthetic methods for the preparation of nicotinamide riboside and related compounds. WO2017218580A1; Gelman, D., A. ZOABI, A. Zeira and R. Abu-Reziq (2017). Process for preparation of nicotinamide riboside (NR) and cosmetic composition comprising NR and a phosphate-binding agent. WO2017145151A1). However, organic synthesis is complex, requiring the addition and removal of protection groups, and using environmentally-unfriendly organic solvents. NR can be synthesized by enzymes pentosyl transferases (Velasquez J E, Green P R, Wos J A (2015) Method for preparing nicotinamide riboside. US20170121746A1), but it requires a lot of methods to shift the equilibrium reactions toward NR for relatively high yields, for example, in situ removal of phosphate ions. It can also be produced by engineered microorganisms (Brenner, C., P. Belenky and K. L. Bogan (2009). Yeast strain and method for using the same to produce nicotinamide riboside. U.S. Pat. No. 8,114,626B2; Lawrence, A. and C. ViAROUGE (2017). Microbial production of nicotinamide riboside. WO2018211051A1), but its titers and yields are low.

NMN can be synthesized from NR by chemical synthesis (Sauve, A. and F. S. Mohammed (2015). Efficient synthesis of nicotinamide mononucleotide. WO2016160524A1), by using an enzyme mutant of phosphoribosylpyrophosphate (PRPP) synthetase (Wu L, Sinclair D A, Meetze K (2015) Enzymatic systems and methods for synthesizing nicotinamide mononucleotide and nicotinic acid mononucleotide. WO2016198948A1.), nicotinamide riboside kinase (Tao A, Fu M, Liang X-L (2016) Method for preparing beta-nicotinamide mononucleotide by using enzymic method. WO2018120069A1, CN201611245619), or a combination of nicotinamide phosphoribosyltransferase, phosphoribose pyrophosphokinase, and ribokinase (Fu R-Z, and Zhang Q (2016) Method for preparing nicotinamide mononucleotide. WO2017185549A1), or by engineered organism containing overexpressed overexpresses an enzyme nicotinamide phosphoribosyltransferase (Sinclair D A, and Ear P H (2014) Biological production of NAD precursors and analogs. US20160287621A1, WO2015069860A1).

NAD was purified from microorganisms but its production costs were very high. It can be overexpressed by fermentation of engineered microorganisms (Sinclair, D A and Ear, P H (2014) Biological production of NAD precursors and analogs. US20160287621A1, WO2015069860A1). A U.S. Pat. No. 4,411,995 (Whitesides, G. and D. R. Walt (1981). Synthesis of nicotinamide cofactors. U.S. Pat. No. 4,411,995) describes an enzymatic process for producing NAD, but such a method, while efficient in its yield, requires carefully controlled conditions and the addition of costly enzymes. Recently, it is mainly produced from NMN and ATP by an NMNAT enzyme (Tao, A., B. Li, X. Ju, X. Liang and J. Zhuang (2013). Method for preparing oxidized coenzyme I. WO2014146250A1) or even one enzyme from a hyper-thermophilic microorganism (Fu, R. Z. (2013). Nicotinamide mononucleotide adenylyltransferase (Nmnat) mutant as well as coding gene and application thereof. CN103710321B). It can also be synthesized from ATP, deamide-NAD and ammonia by the Bacillus stearothermophilus NAD synthetase (EC 6.3.1.5) (Takahashi, M., H. Misaki, S. Imamura and K. Matsuura (1987). “Novel NAD synthetase, assay method using said novel NAD synthetase and a process for production thereof. U.S. Pat. No. 4,921,786A). It also can be synthesized by using an in vitro reconstituted mammalian NAD biosynthesis system comprising of NAMPT and NMNAT from NAM, PRPP and ATP (Imai, S.-I., J. R. Revollo and A. A. Grimm (2005). NAD biosynthesis systems. US20090246803A1. WO2006041624A2). NADP can be synthesized with ATP or polyphosphate and NAD by using NAD kinase (Kawai, S., K. Murata, H. Matsukawa, S. Tomisako, Y. Ando and Y. Matsuo (2001). Process for preparing nicotinamide adenine dinucleotide phosphate (NADP). U.S. Pat. No. 7,863,014B2; Tao, A., B. Li, L. Xie, J. Zhuang, Y. Zhou, C. Zhang and G. Liu (2012). Enzymatic preparation method of oxidized coenzyme II. CN102605027B).

To make NMN and its derivatives NAD and NADP at low costs and in high yields, the present invention was demonstrated to address the below issues:
(1) make less-costly precursor D-ribose 1-phosphate from low-cost abundant feedstocks;
(2) push the artificial enzymatic pathways toward the desired products by in situ removing by-products by adding their respective enzymes and/or choosing the irreversible reactions at the last biochemical reactions;
(3) regenerate ATP from less costly substrates, such as acetyl phosphate and polyphosphate, and/or decrease the use of ATP as the substrate; and
(4) make the less-costly precursor 5-phospho-alpha-D-ribose-1-diphosphate (PRPP) from low-cost nucleotides.

### SUMMARY OF THE INVENTION

The present invention utilizes available and low-cost starting materials, isolated enzymes, either freely soluble or immobilized for stability and recoverable for reuse, or microorganisms containing said enzymes, and allows for the integrated multiple-enzyme reactions (called artificial enzymatic pathways in one pot or multiple-enzyme one pot) to push the overall reaction toward the synthesis of desired products in high yields.

In one embodiment, NMN can be synthesized from nicotinamide (NAM) and D-ribose-1-phosphate (R1P) catalyzed by two enzymes—nicotinamide riboside phosphorylase (NRP, EC 2.4.2.1) and nicotinamide riboside kinase (NRK, EC 2.7.1.22), along with ATP regeneration (FIG. 2). The cost-effective synthesis of NMN needs two inputs: R1P and ATP. R1P can be produced from substrates via a lot of enzymatic pathways (FIGS. 3-7). ATP can be regenerated via a few approaches (FIG. 8) (Chen, H.-G., and Zhang, Y.-H. P. J. (2020) “Enzymatic Regeneration and Conservation of ATP: Challenges and Opportunities” Critical Review in Biotechnology, Epub, doi.org/10.1080/07388551.07382020.01826403). The consolidation of Reactions 1 and 2 as well as in vitro R1P generation and ATP regeneration (FIG. 2) can have relatively high NMN yields because the last unidirectional reaction (Reaction 2) can push the synthesis of NMN forward.

In one embodiment, alpha-D-ribose-1-phosphate (R1P) can be generated from purine nucleosides (e.g., inosine, guanosine and adenosine) catalyzed by purine nucleoside phosphorylase (PNP) and/or guanosine nucleoside phosphorylase (GP, 2.4.2.15) (FIG. 3). Purine nucleosides and their phosphorylases may be replaced with pyrimidine nucleosides (e.g., urdine or thymidine) and pyrimidine-nucleoside phosphorylase (PyNP, EC 2.4.2.2), uridine phosphorylase (UP, EC 2.4.2.3) or thymidine phosphorylase (TP, EC 2.4.2.4), respectively.

In one embodiment, R1P can be generated from nucleotides (e.g., inosine monophosphate (IMP), guanosine monophosphate (GMP), or adenosine monophosphate (AMP)) catalyzed by pyrimidine/purine nucleotide 5′-monophosphate nucleosidase (PPMPN, EC 3.2.2.10) and phosphopentomutase (PPM, EC 5.4.2.7) (FIG. 4). PPMPN may be replaced with inosinate nucleosidase (EC 3.2.2.12) or AMP nucleosidase (EC 3.2.2.4) or their mixture.

In one embodiment, R1P can be generated from D-ribose and ATP catalyzed by ribokinase (RK, EC 2.7.1.15) and phosphopentomutase (PPM, EC 5.4.2.7) (FIG. 5 step a), along with ATP regeneration (FIG. 8).

In one embodiment, R1P can be generated from D-xylose, which can be converted to D-ribose by D-xylose isomerase (D-XI, EC 5.3.1.5), pentose 3-epimerase (P3E, EC 5.1.3.31), and D-ribose isomerase (D-RI, EC 5.3.1.20) or mannose 6-phosphate isomerase (MPI, EC 5.3.1.8) (FIG. 5 step b), followed enzymatic catalysis by RK and PPM (FIG. 5 step a).

In one embodiment, R1P can be generated from D-xylose and polyphosphate (FIG. 6), whereas the artificial enzymatic pathway is comprised of D-xylose isomerase (D-XI, EC 5.3.1.5), polyphosphate xylulokinase (XK, EC 2.7.1.17), D-xylulose 5-phosphate 3-epimerase (RuPE, EC 5.1.3.1), ribose-5-phosphate isomerase (RPI, EC 5.3.1.6), and phosphopentomutase (PPM, EC 5.4.2.7).

In one embodiment, R1P can be generated from starch and phosphate (FIG. 7), whereas the artificial enzymatic pathway is comprised of αGP (alpha-glucan phosphorylase, EC 2.4.1.1), PGM (phosphoglucomutase, EC 5.4.2.2), G6PDH (glucose-6-phosphate dehydrogenase, EC 1.1.1.49), 6-phosphogluconolactonase (6PGL, EC 3.1.1.31), 6PGDH (6-phosphogluconate dehydrogenase, EC 1.1.1.44), RPI (ribose 5-phosphate isomerase, EC 5.3.1.6), PPM (phosphopentomutase, EC 5.4.2.7), and two reduced NAD(P)H may be removed by H₂O-forming NAD(P)H oxidase (NOX, EC 1.6.3.4 or EC 1.6.3.2).

In one embodiment, R1P can be generated from cellodextrin/cellobiose and phosphate (FIG. 7), whereas the artificial enzymatic pathway is comprised of CDP (cellodextrin phosphorylase, EC 2.4.1.49), CBP (cellobiose phosphorylase, EC 2.4.1.20), PGM, G6PDH, 6PGL, 6PGDH, RPI, PPM, and two reduced NAD(P)H may be removed by NOX.

In one embodiment, R1P can be generated from sucrose and phosphate (FIG. 7), whereas the artificial enzymatic pathway is comprised of SP (sucrose phosphorylase, EC 2.4.1.7), PGM, G6PDH, 6PGL, 6PGDH, RPI, PPM, and two reduced NAD(P)H may be removed by NOX.

In one embodiment, R1P can be generated from D-glucose and polyphosphate (FIG. 7), whereas the artificial enzymatic pathway is comprised of PPGK (polyphosphate glucokinase, EC 2.7.1.63), G6PDH, 6PGL, 6PGDH, RPI, PPM, and two reduced NAD(P)H may be removed by NOX.

In one embodiment, R1P can be generated from D-fructose and polyphosphate (FIG. 7), whereas the artificial enzymatic pathway is comprised of D-XI (xylose isomerase, EC 5.3.1.5), PPGK, G6PDH, 6PGL, 6PGDH, RPI, PPM, and two reduced NAD(P)H may be removed by NOX.

In one embodiment, R1P can be generated from a mixture of hexoses and phosphate/or polyphosphate, with a mixture of the above enzymes (FIG. 7).

In one embodiment, ATP can be regenerated from acetyl phosphate catalyzed by acetate kinase (AK, EC 2.7.2.1) (FIG. 8 step a), where the ATP consumption example was the synthesis of NMN from NR catalyzed by NR kinase (NRK, EC 2.7.1.22). ATP consumption example is not limited to the reaction catalyzed by NRK.

In one embodiment, ATP can be recycled for in-depth use by using adenylate kinase (ADK, EC 2.7.4.3) and adenosine kinase (AdK, EC 2.7.1.20) (FIG. 8 step b), where the ATP consumption example was the synthesis of NMN from NR catalyzed by NR kinase (NRK, EC 2.7.1.22). ATP consumption example is not limited to the reaction catalyzed by NRK.

In one embodiment, adenosine is hydrolyzed to adenine and ribose catalyzed by adenosine nucleosidase (AN, EC 3.2.2.7) or purine nucleosidase (PN, EC 3.2.2.1).

In one embodiment, ATP can be regenerated from polyphosphate by using polyphosphate kinase (PPK, EC 2.7.4.1) (FIG. 8 step c), where the ATP consumption example was the synthesis of NMN from NR catalyzed by NR kinase (NRK, EC 2.7.1.22). ATP consumption example is not limited to the reaction catalyzed by NRK.

In one embodiment, ATP can be regenerated from polyphosphate by using polyphosphate: AMP phosphotransferase (PPT, EC 2.7.4.B2) and adenylate kinase (ADK, EC 2.7.4.3) (FIG. 8 step d), where the ATP consumption example was the synthesis of NMN from NR catalyzed by NR kinase (NRK, EC 2.7.1.22). ATP consumption example is not limited to the reaction catalyzed by NRK.

In one embodiment, ATP can be regenerated by using permeabilized cells (e.g., Escherichia coli, Saccharomyces cerevisiae) (FIG. 8 step e), where the ATP consumption example was the synthesis of NMN from NR catalyzed by NR kinase (NRK, EC 2.7.1.22). ATP consumption example is not limited to the reaction catalyzed by NRK.

In one embodiment, ATP can be regenerated by using artificial or natural organelles (e.g., light-driven chloroplasts and mitochondria) that continuously make ATP.

In one embodiment, NMN can be synthesized from inosine monophosphate (IMP) and nicotinamide (NAM) catalyzed by two enzymes: hypoxanthine/guanine phosphoribosyltransferase (HGPRT, EC 2.4.2.8) and nicotinamide phosphoribosyltransferase (NAMPT, EC 2.4.2.12) (FIG. 9). The consolidation of Reactions 3 and 4 in one pot can recycle PRPP and diphosphate (PPᵢ), resulting in Reaction 5.

IMP+NAM [symbol] NMN+hypoxanthine [5]

In one embodiment, NMN can be synthesized from guanosine monophosphate (GMP) and nicotinamide (NAM) catalyzed by two enzymes: hypoxanthine/guanine phosphoribosyltransferase (HGPRT, EC 2.4.2.8) and nicotinamide phosphoribosyltransferase (NAMPT, EC 2.4.2.12) (FIG. 9). Similar to Reaction 5, the consolidation of Reactions 3 and the said reaction in one pot can recycle PRPP and diphosphate (PPᵢ), resulting in Reaction 6.

GMP+NAM [symbol] NMN+guanosine [6]

In one embodiment, NAD can be synthesized from NMN and ATP catalyzed by nicotinamide nucleotide adenylyltransferase (NMNAT, EC 2.7.7.1) with a removal of diphosphate by diphosphatase (DPP, EC 3.6.1.1) (FIG. 10). NMNAT may be replaced with nicotinate-nucleotide adenylyltransferase (EC 2.7.7.18).

In one embodiment, NADP can be synthesized from NAD and ATP or polyphosphate catalyzed by ATP-dependent or polyphosphate-dependent NAD kinase (NADK, EC 2.7.1.23) (FIG. 10).

In one embodiment, NADP can be synthesized from NMN and ATP/polyphosphate catalyzed by NMNAT, DPP, and NADK (FIG. 10).

In one embodiment, NR can be hydrolyzed from NMN, cogenerating Pᵢ, catalyzed by 5′-nucleotidase (EC 3.1.3.5), nucleotide phosphatase (EC 3.1.3.31), or acid phosphatase (EC 3.1.3.2).

In one embodiment, adenosine can be converted to adenine and ribose by using adenosine nucleosidase (AN, EC 3.2.2.7) or purine nucleosidase (PN, EC 3.2.2.1) (FIG. 11 step a).

In one embodiment, guanine can be converted to urate by using guanine deaminase (GDA, EC 3.5.4.3), xanthine oxidase (XO, EC 1.17.3.2), and catalase (CA, EC 1.11.1.7, EC 1.11.1.21) (FIG. 11 step b).

In one embodiment, hypoxanthine can be converted to xanthine and urate by using XO and CA (FIG. 11 step c).

In one embodiment, hypoxanthine can be converted to xanthine by xanthine dehydrogenase (XDH, EC 1.17.1.4), H₂O-forming NADH oxidase (NOX, EC 1.6.3.4 or EC 1.6.3.2) (FIG. 11 step d). NOX may be replaced with other NADH-consuming enzymes, for example, hydrogenase, H₂O₂ formation NADH oxidase (EC 1.6.3.3).

In one embodiment, diphosphate can be converted to two phosphate ions by (inorganic) diphosphatase (DPP, EC 3.6.1.1).

In one embodiment, NMN can be synthesized from NAM, IMP and acetyl-phosphate catalyzed by five enzymes: inosinate nucleosidase (IMPN, EC 3.2.2.12), PPM, NRP, NRK, and AK (FIG. 12). The substrate IMP and its respective enzyme IMPN may be replaced with GMP and pyrimidine/purine nucleotide monophosphate nucleosidase (PPMPN, EC 3.2.2.10), respectively.

In one embodiment, NMN can be synthesized from NAM, IMP and ATP catalyzed by eight enzymes: IMPN, PPM, NRP, NRK, ADK, AdK, NO, and CA (FIG. 13). The substrate IMP and its respective enzyme IMPN may be replaced with GMP and pyrimidine/purine nucleotide monophosphate nucleosidase (PPMPN, EC 3.2.2.10), respectively. To improve NMN yield, hypoxanthine could be removed by the addition of more enzymes as shown in FIG. 11 steps c & d.

For example, two supplementary enzymes may be xanthine dehydrogenase (XDH) and H₂O-forming NADH oxidase (NOX).

In one embodiment, NMN can be synthesized from NAM, IMP and polyphosphate catalyzed by five enzymes: IMPN, PPM, NRP, NRK, PPK (FIG. 14). The substrate IMP and its respective enzyme IMPN may be replaced with GMP and PPMPN, respectively. To improve NMN yield, hypoxanthine could be removed by the addition of more enzymes as shown in FIG. 11 steps c & d. For example, two supplementary enzymes may be xanthine dehydrogenase (XDH) and H₂O-forming NADH oxidase (NOX).

In one embodiment, NAD can be synthesized from NAM, IMP and ATP catalyzed by IMPN, PPM, NRP, NRK, NMNAT and DPP (FIG. 15). Also, a bifunctional enzyme consists of NRK and NMNAT. The substrate IMP and its respective enzyme IMPN may be replaced with GMP and PPMPN, respectively. To improve NMN yield, hypoxanthine could be removed by the addition of more enzymes as shown in FIG. 11 steps c & d. For example, two supplementary enzymes may be xanthine dehydrogenase (XDH) and H₂O-forming NADH oxidase (NOX).

In one embodiment, NMN can be synthesized from IMP and NAM catalyzed by four enzymes: hypoxanthine/guanine phosphoribosyltransferase (HGPRT, EC 2.4.2.8), nicotinamide phosphoribosyltransferase (NAMPT, EC 2.4.2.12), xanthine oxidase (XO), and catalase (CA) (FIG. 16).

In one embodiment, NMN can be synthesized from IMP and NAM catalyzed by four enzymes: HGPRT, NAMPT, xanthine dehydrogenase (XDH), and H₂O-forming NADH oxidase (NOX) (FIG. 17). NAD may be replaced with NADP. NOX may be replaced by NAD(P)H-consuming enzymes, for example, hydrogenase, H₂O₂-forming NAD(P)H oxidase, etc.

In one embodiment, NMN can be synthesized from GMP and NAM catalyzed by five enzymes: HGPRT, NAMPT, guanine deaminase (GDA, EC 3.5.4.3), XO, and CA (FIG. 18).

In one embodiment, NAD can be synthesized from IMP, ATP, and NAM catalyzed by five enzymes: hypoxanthine/guanine phosphoribosyltransferase (HGPRT, EC 2.4.2.8), nicotinamide phosphoribosyltransferase (NAMPT, EC 2.4.2.12), nicotinamide nucleotide adenylyltransferase (NMNAT, EC 2.7.7.1), XDH and NOX (FIG. 19). NMNAT may be replaced with nicotinate-nucleotide adenylyltransferase (EC 2.7.7.18). Diphosphatase (DPP) may be added to catalyze diphosphate to two phosphate.

In one embodiment, NAD can be synthesized from GMP, ATP, and NAM catalyzed by six enzymes: HGPRT, NAMPT, NMNAT, GDA, XO and CA (FIG. 20). NMNAT may be replaced with nicotinate-nucleotide adenylyltransferase. Diphosphatase (DPP) may be added to catalyze diphosphate to two phosphate.

In one embodiment, NADP can be synthesized from IMP (or GMP), ATP, NAM and polyphosphate catalyzed by four enzymes: HGPRT, NAMPT, NMNAT, and NADK (FIG. 21). Also, guanine can be removed by using GDA, XO and CA; hypoxanthine can be removed by using XDH and NOX or XO and CA. Diphosphatase (DPP) may be added to catalyze diphosphate to two phosphate ions. Polyphosphate-dependent NADK may be replaced with ATP-dependent NADK supplemented with ATP regeneration system (FIG. 8).

One-pot biosynthesis comprised of multiple enzymes and even artificial enzymatic pathways is an emerging biomanufacturing platform. The integration of numerous enzymes in one pot or bioreactor or vessel consolidate multiple-step bioreactions, having multiple benefits, such as no separation of intermediates, enhanced volumetric productivity. But the careful design of pathways featuring the unidirectional last reaction step or effectively complete removal of byproducts by unidirectional reactions can increase the yields of desired products.

### BRIEF DESCRIPTION OF THE DRAWINGS

FIG. 1. Names and structures of vitamin B3 (i.e., niacin (NA) and nicotinamide (NAM)) and their derivatives (nicotinamide riboside (NR), nicotinamide mononucleotide (NMN), nicotinamide adenine dinucleotide (NAD), and nicotinamide adenine dinucleotide phosphate (NADP)).

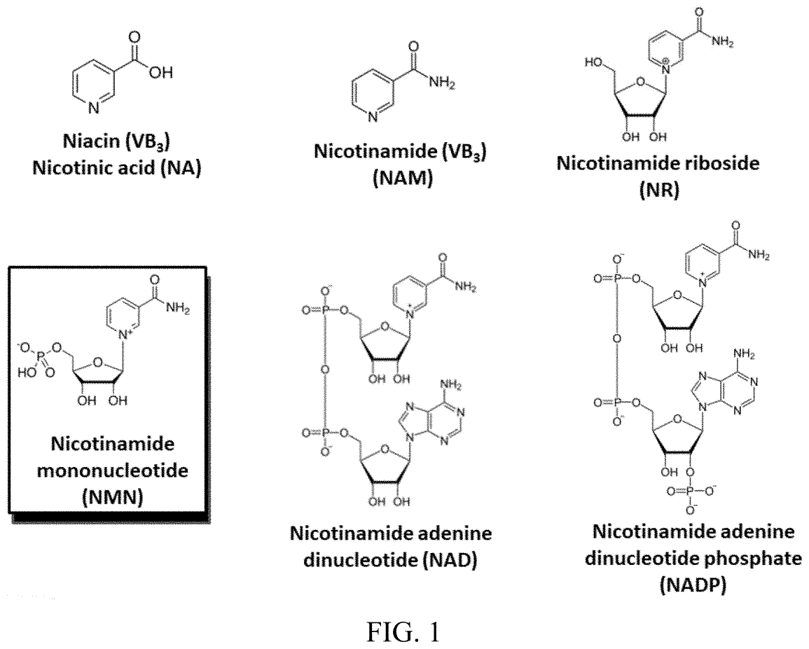

FIG. 2. Scheme of one-pot NMN synthesis from D-ribose 1-phosphate (R1P) and NAM catalyzed by nicotinamide riboside phosphorylase (NRP) and nicotinamide riboside kinase (NRK, EC 2.7.1.22) plus two supporting systems (i.e., in vitro R1P generation and ATP regeneration). NRP may be replaced with purine nucleoside phosphorylase (PNP, EC 2.4.2.1) or guanosine phosphorylase (GP, EC 2.4.2.15) or pyrimidine nucleoside phosphorylase (EC 2.4.2.2) or uridine phosphorylase (UP, EC 2.4.2.3) or thymidine phosphorylase (TP, EC 2.4.2.4); NRK may be replaced with nicotinate riboside kinase (NaRK, EC 2.7.1.173).

FIG. 3. Scheme of the in vitro generation of R1P from nucleosides (e.g., inosine, guanosine and adenosine) catalyzed by purine nucleoside phosphorylase (PNP, EC 2.4.2.1) and/or guanosine nucleoside phosphorylase (GP, EC 2.4.2.5). Purine nucleosides and their phosphorylases may be replaced with pyrimidine nucleosides and pyrimidine-nucleoside phosphorylase (PyNP, EC 2.4.2.2), uridine phosphorylase (UP, EC 2.4.2.3) or thymidine phosphorylase (TP, EC 2.4.2.4), respectively.

FIG. 4. Scheme of the in vitro generation of R1P from nucleotides (e.g., inosine monophosphate (IMP), guanosine monophosphate (GMP), or adenosine monophosphate (AMP)) catalyzed by pyrimidine/purine nucleotide 5′-monophosphate nucleosidase (PPMPN, EC 3.2.2.10) and phosphopentomutase (PPM, EC 5.4.2.7). PPMPN may be replaced with inosinate nucleosidase (EC 3.2.2.12) or AMP nucleosidase (EC 3.2.2.4).

FIG. 5. Scheme of the in vitro generation of R1P from (a) D-ribose catalyzed by ribokinase (RK, EC 2.7.1.15) and phosphopentomutase (PPM, EC 5.4.2.7), along with ATP regeneration and from (b) D-xylose to D-ribose catalyzed by three enzymes: D-xylose isomerase (D-XI, EC 5.3.1.5), pentose 3-epimerase (P3E, EC 5.1.3.31), and D-ribose isomerase (D-RI, EC 5.3.1.20) or mannose 6-phosphate isomerase (MPI, EC 5.3.1.8).

FIG. 6. Scheme of the in vitro generation of R1P from D-xylose and polyphosphate catalyzed by the enzymes: D-xylose isomerase (D-XI, EC 5.3.1.5), polyphosphate xylulokinase (XK, EC 2.7.1.17), D-xylulose 5-phosphate 3-epimerase (RuPE, EC 5.1.3.1), ribose-5-phosphate isomerase (RPI, EC 5.3.1.6), and phosphopentomutase (PPM, EC 5.4.2.7).

FIG. 7. Scheme of the in vitro generation of R1P from hexoses (e.g., starch, sucrose, cellodextrins, glucose, fructose) via ATP-free substrate phosphorylation followed by a partial pentose phosphate pathway with cogeneration of CO₂ and 2 NAD(P)H, which can be removed by H₂O-forming NAD(P)H oxidase (NOX, EC 1.6.3.4 or EC 1.6.3.2). The enzymes are αGP (alpha-glucan phosphorylase, EC 2.4.1.1), SP (sucrose phosphorylase, EC 2.4.1.7), CDP (cellodextrin phosphorylase, EC 2.4.1.49), CBP (cellobiose phosphorylase, EC 2.4.1.20), PPGK (polyphosphate glucokinase, EC 2.7.1.63), D-XI (xylose isomerase, EC 5.3.1.5), PGM (phosphoglucomutase, EC 5.4.2.2), G6PDH (glucose-6-phosphate dehydrogenase, EC 1.1.1.49), 6-phosphogluconolactonase (6PGL, EC 3.1.1.31), 6PGDH (6-phosphogluconate dehydrogenase, EC 1.1.1.44), RPI (ribose 5-phosphate isomerase, EC 5.3.1.6), and PPM (phosphopentomutase, EC 5.4.2.7).

FIG. 8. Scheme of the representative ATP regeneration from acetyl-phosphate catalyzed by acetate kinase (AK, EC 2.7.2.1) (a); from ATP catalyzed by adenylate kinase (ADK, EC 2.7.4.3) and adenosine kinase (AdK, EC 2.7.1.20) (b); from polyphosphate catalyzed by polyphosphate kinase (PPK, EC 2.7.4.1) (c); from polyphosphate catalyzed by polyphosphate: AMP phosphotransferase (PPT, EC 2.7.4.B2) and adenylate kinase (ADK, EC 2.7.4.3) (d); and from permeabilized cells (e).

FIG. 9. Scheme of synthesis of NMN from inosine monophosphate (IMP) or GMP catalyzed by hypoxanthine/guanine phosphoribosyltransferase (HGPRT, EC 2.4.2.8) and nicotinamide phosphoribosyltransferase (NAMPT, EC 2.4.2.12), where PRPP is 5-phospho-alpha-D-ribose 1-diphosphate. The substrate IMP or GMP and its respective enzyme HGPRT may be replaced with AMP and adenine phosphoribosyltransferase (APRT, EC 2.4.2.7), respectively.

FIG. 10. Scheme of the synthesis of NMN-derivatives: NAD production from NMN catalyzed by nicotinamide nucleotide adenylyltransferase (NMNAT, EC 2.7.7.1) with a removal of diphosphate by diphosphatase (DPP, EC 3.6.1.1) (a); and NADP production from NAD catalyzed by ATP-dependent or polyphosphate-dependent NAD kinase (NADK, EC 2.7.1.23) (b).

FIG. 11. Scheme of efficient removal of undesired products: adenosine by using adenosine nucleosidase (AN, EC 3.2.2.7) (a); guanine deaminase (GDA, EC 3.5.4.3), xanthine oxidase (XO, EC 1.17.3.2), and catalase (CA, EC 1.11.1.6, EC 1.11.1.21, or EC 1.11.1.7) (b); hypoxanthine by XO and CA (c); and hypoxanthine by xanthine dehydrogenase (XDH, EC 1.17.1.4) and H₂O-forming NADH oxidase (NOX, EC 1.6.3.4 or EC 1.6.3.2) (d). AN may be replaced with nucleoside oxidase (NO, EC 1.1.3.39) and catalase (CA) or purine nucleosidase (PN, EC 3.2.2.1).

FIG. 12. Scheme of one-pot synthesis of NMN from NAM, IMP and acetyl-phosphate catalyzed by five enzymes: inosinate nucleosidase (IMPN, EC 3.2.2.12), phosphopentomutase (PPM, EC 5.4.2.7), nicotinamide riboside phosphorylase (NRP, EC 2.4.2.1), nicotinamide riboside kinase (NRK, EC 2.7.1.22), and acetate kinase (AK, EC 2.7.2.1). The substrate IMP and its respective enzyme IMPN may be replaced with GMP and pyrimidine/purine nucleotide monophosphate nucleosidase (PPMPN, EC 3.2.2.10), respectively.

FIG. 13. Scheme of one-pot synthesis of NMN from NAM, IMP and ATP catalyzed by seven enzymes: inosinate nucleosidase (IMPN, EC 3.2.2.12), phosphopentomutase (PPM, EC 5.4.2.7), nicotinamide riboside phosphorylase (NRP), nicotinamide riboside kinase (NRK, EC 2.7.1.22), adenylate kinase (ADK, EC 2.7.4.3), adenosine kinase (AdK, EC 2.7.1.20), and adenosine nucleosidase (AN, EC 3.2.2.7). The substrate IMP and its respective enzyme IMPN may be replaced with GMP and PPMPN, respectively. AN may be replaced with nucleoside oxidase (NO, EC 1.1.3.39) and catalase (CA) or purine nucleosidase (PN, EC 3.2.2.1).

FIG. 14. Scheme of one-pot synthesis of NMN from NAM, IMP and polyphosphate catalyzed by five enzymes: inosinate nucleosidase (IMPN, EC 3.2.2.12), phosphopentomutase (PPM, EC 5.4.2.7), nicotinamide riboside phosphorylase (NRP), nicotinamide riboside kinase (NRK, EC 2.7.1.22), and polyphosphate kinase (PPK, EC 2.7.4.1). The substrate IMP and its respective enzyme IMPN may be replaced with GMP and PPMPN, respectively. To improve NMN yield, hypoxanthine could be removed by the addition of more enzymes as shown in FIG. 11 steps c&d. For example, two supplementary enzymes may be xanthine dehydrogenase (XDH) and H₂O-forming NADH oxidase (NOX).

FIG. 15. Scheme of one-pot synthesis of NAD from NAM, IMP, and ATP catalyzed by six enzymes: inosinate nucleosidase (IMPN, EC 3.2.2.12), phosphopentomutase (PPM, EC 5.4.2.7), nicotinamide riboside phosphorylase (NRP), nicotinamide riboside kinase (NRK, EC 2.7.1.22), nicotinamide nucleotide adenylyltransferase (NMNAT, EC 2.7.7.1), and diphosphatase (DPP, EC 3.6.1.1). Also, a bifunctional enzyme consists of NRK and NMNAT. The substrate IMP and its respective enzyme IMPN may be replaced with GMP and PPMPN, respectively. To improve NMN yield, hypoxanthine could be removed by the addition of more enzymes as shown in FIG. 11 steps c&d. For example, two supplementary enzymes may be xanthine dehydrogenase (XDH) and H₂O-forming NADH oxidase (NOX).

FIG. 16. Scheme of one-pot ATP-free synthesis of NMN from IMP and NAM catalyzed by four enzymes: hypoxanthine/guanine phosphoribosyltransferase (HGPRT, EC 2.4.2.8), nicotinamide phosphoribosyltransferase (NAMPT, EC 2.4.2.12), xanthine oxidase (XO), and catalase (CA).

FIG. 17. Scheme of one-pot ATP-free synthesis of NMN from IMP and NAM catalyzed by four enzymes: hypoxanthine/guanine phosphoribosyltransferase (HGPRT, EC 2.4.2.8), nicotinamide phosphoribosyltransferase (NAMPT, EC 2.4.2.12), xanthine dehydrogenase (XDH), and H₂O-forming NADH oxidase (NOX). NAD may be replaced with NADP. NOX may be replaced by NAD(P)H-consuming enzymes, for example, hydrogenase, H₂O₂-forming NAD(P)H oxidase, etc.

FIG. 18. Scheme of one-pot ATP-free synthesis of NMN from GMP and NAM catalyzed by five enzymes: hypoxanthine/guanine phosphoribosyltransferase (HGPRT, EC 2.4.2.8), nicotinamide phosphoribosyltransferase (NAMPT, EC 2.4.2.12), guanine deaminase (GDA, EC 3.5.4.3), XO, and CA.

FIG. 19. Scheme of one-pot one-ATP-use synthesis of NAD from IMP, ATP, and NAM catalyzed by five enzymes: hypoxanthine/guanine phosphoribosyltransferase (HGPRT, EC 2.4.2.8), nicotinamide phosphoribosyltransferase (NAMPT, EC 2.4.2.12), nicotinamide nucleotide adenylyltransferase (NMNAT, EC 2.7.7.1), xanthine dehydrogenase (XDH), and H₂O-forming NADH oxidase (NOX). NMNAT may be replaced with nicotinate-nucleotide adenylyltransferase (EC 2.7.7.18). Diphosphatase (DPP) may be added to catalyze diphosphate to two phosphate.

FIG. 20. Scheme of one-pot synthesis of NAD from GMP, ATP, and NAM catalyzed by six enzymes: hypoxanthine/guanine phosphoribosyltransferase (HGPRT, EC 2.4.2.8), nicotinamide phosphoribosyltransferase (NAMPT, EC 2.4.2.12), nicotinamide nucleotide adenylyltransferase (NMNAT, EC 2.7.7.1), GDA, XO and CA. NMNAT may be replaced with nicotinate-nucleotide adenylyltransferase (EC 2.7.7.18). Diphosphatase (DPP) may be added to catalyze diphosphate to two phosphate.

FIG. 21. Scheme of one-pot synthesis of NADP from IMP (or GMP), ATP, NAM and polyphosphate catalyzed by four enzymes: hypoxanthine/guanine phosphoribosyltransferase (HGPRT, EC 2.4.2.8), nicotinamide phosphoribosyltransferase (NAMPT, EC 2.4.2.12), nicotinamide nucleotide adenylyltransferase (NMNAT, EC 2.7.7.1), polyphosphate-dependent NAD kinase (NADK, EC 2.7.1.23). Guanine can be removed by using GDA, XO and CA. Hypoxanthine can be removed by using XDH/NOX or XO/CA. Diphosphatase (DPP) may be added to catalyze diphosphate to two phosphate. Polyphosphate-dependent NADK may be replaced with ATP-dependent NADK supplemented with ATP regeneration system (FIG. 8).

### DETAILED DESCRIPTION OF THE INVENTION

### I. Definitions

As used herein, “artificial enzymatic pathway” refers to the manmade enzyme mixture, whereas the product of one enzyme is the substrate of another enzyme. Via the intermediates (i.e., the product of one enzyme and the substrate of another enzyme), several enzymes make up the artificial enzymatic pathway.

As used herein, “one pot” or “one vessel” refers to the multiple-step bioreactions catalyzed by multiple enzymes or the artificial enzymatic pathway occurring in one bioreactor or even in one microbial cell (also called a microbioreactor), whereas they work together in trade-off reaction conditions for all of enzyme components. Sometimes, it was called the consolidated bioreactions in one pot.

As used herein, the term “nucleoside” refers to a glycosylamine having a nitrogenous base, such as a purine or pyrimidine, linked to a 5-carbon sugar (e.g. D-ribose or 2-deoxy-D-ribose) via a β-glycosidic linkage. Nucleosides are also referred as “ribonucleosides” when the sugar moiety is D-ribose, for example, inosine, guanosine, adenosine, nicotinamide riboside.

As used herein, the term “purine nucleoside” refers to a nucleoside, wherein the nitrogenous base is a purine.

As used herein, the term “pyrimidine nucleoside” refers to a nucleoside, wherein the nitrogenous base is a pyrimidine.

As used herein, the term “nucleotide”, also known as “nucleoside monophosphate”, refers to a compound having a nucleoside esterified to an orthophosphate group via the hydroxyl group bound to the 5-carbon of the sugar moiety. Nucleotides are also referred as “ribonucleotides”. Non-limiting examples of nucleotide include, but not limited to for example, inosine monophosphate (IMP), guanosine monophosphate (GMP), adenosine monophosphate (AMP), nicotinamide mononucleoside (NMN).

As used herein, the term “nitrogenous base”, refers to a compound containing a nitrogen atom that has the chemical properties of a base. Non-limiting examples of nitrogenous bases are compounds comprising pyridine, purine, or pyrimidine moieties, including, but not limited to adenine, guanine, hypoxanthine, thymine, cytosine, uracil, and nicotinamide.

As used herein, the term “inorganic orthophosphate” refers to a compound composed of four oxygen atoms arranged in an almost regular tetrahedral array about a central phosphorus atom. Inorganic orthophosphate or phosphate or phosphate ions may be present in several ionic forms, depending on the pH of the solution, including [PO₄]³⁻, [HPO₄]²⁻, [H₂PO₄]⁻, and H₃PO₄.

As used herein, the term “inorganic diphosphate”, also known as “inorganic pyrophosphate”, “pyrophosphate”, “PPᵢ” refers to a compound containing one P—O—P bond generated by corner sharing of two PO₄ tetrahedra. Inorganic diphosphate may be present in several ionic forms, depending on the pH of the solution, including [P₂O₇]⁴⁻, [HP₂O₇]³⁻, [H₂P₂O₇]²⁻, [H₃P₂O₇]⁻, and H₄P₂O₇.

As used herein, the term “conservative substitution” refers to the substitution in a polypeptide of an amino acid with a functionally similar amino acid. A conservative substitution involves replacement of an amino acid residue with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), β-branched side chains (e.g., threonine, valine, and isoleucine) and aromatic side chains (e.g., tyrosine, phenylalanine, tryptophan, and histidine).

As used herein, the term “converting” refers to a chemical transformation from one molecule to another, primarily catalyzed by an enzyme or enzymes, although other organic or inorganic catalysts may be used.

As used herein, the term “conversion,” in the context of chemical transformations, refers to the ratio in % between the molar amount of the desired product and the molar amount of the limiting reagent.

As used herein, the term “endogenous” refers to polynucleotides, polypeptides, or other compounds that are expressed naturally or originate within an organism or cell. That is, endogenous polynucleotides, polypeptides, or other compounds are not exogenous. For instance, an “endogenous” polynucleotide or peptide is present in the cell when the cell was originally isolated from nature.

As used herein, the term “exogenous” refers to any polynucleotide or polypeptide that is not naturally found or expressed in the particular cell or organism where expression is desired. Exogenous polynucleotides, polypeptides, or other compounds are not endogenous.

As used herein, the term “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

As used herein, the term “isolated enzyme” refers to enzymes free of a living organism. Isolated enzymes of the invention may be suspended in solution following lysing of the cell they were expressed in, partially or highly purified, soluble or bound to an insoluble matrix.

As used herein, the term “naturally-occurring” refers to an object that can be found in nature. For example, a polypeptide or polynucleotide sequence that is present in an organism (including viruses) that can be isolated from a source in nature and which has not been intentionally modified by man in the laboratory is naturally-occurring. As used herein, “naturally-occurring” and “wild-type” are synonyms.

As used herein, a recombinant gene that is “over-expressed” produces more RNA and/or protein than a corresponding naturally-occurring gene in the microorganism. Methods of measuring amounts of RNA and protein are known in the art. Over-expression can also be determined by measuring protein activity such as enzyme activity. Depending on the embodiment of the invention, “over-expression” is an amount at least 3%, at least 5%, at least 10%, at least 20%, at least 25%, or at least 50% more. An over-expressed polynucleotide is generally a polynucleotide native to the host cell, the product of which is generated in a greater amount than that normally found in the host cell. Over-expression is achieved by, for instance and without limitation, operably linking the polynucleotide to a different promoter than the polynucleotide's native promoter or introducing additional copies of the polynucleotide into the host cell.

As used herein, the term “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T”. Put another way, “polynucleotide” refers to a polymer of nucleotides removed from other nucleotides (a separate fragment or entity) or can be a component or element of a larger nucleotide construct, such as an expression vector or a polycistronic sequence. Polynucleotides include DNA, RNA and cDNA sequences.

As used herein, the term “polypeptide” refers to a polymer composed of amino acid residues which may or may not contain modifications such as phosphates and formyl groups.

As used herein, “recombinant polynucleotide” refers to a polynucleotide having sequences that are not joined together in nature. A recombinant polynucleotide may be included in a suitable vector, and the vector can be used to transform a suitable host cell. A host cell that comprises the recombinant polynucleotide is referred to as a “recombinant host cell.” The polynucleotide is then expressed in the recombinant host cell to produce, e.g., a “recombinant polypeptide”.

As used herein, the term “recombinant expression vector” refers to a DNA construct used to express a polynucleotide that, e.g., encodes a desired polypeptide. A recombinant expression vector can include, for example, a transcriptional subunit comprising: i) an assembly of genetic elements having a regulatory role in gene expression, for example, promoters and enhancers; ii) a structural or coding sequence which is transcribed into mRNA and translated into protein; and iii) appropriate transcription and translation initiation and termination sequences. Recombinant expression vectors are constructed in any suitable manner. The nature of the vector is not critical, and any vector may be used, including plasmid, virus, bacteriophage, and transposon. Possible vectors for use in the invention include, but are not limited to, chromosomal, nonchromosomal and synthetic DNA sequences, e.g., bacterial plasmids; phage DNA; yeast plasmids; and vectors derived from combinations of plasmids and phage DNA, DNA from viruses such as vaccinia, adenovirus, fowl pox, baculovirus, SV40, and pseudorabies.

As used herein, a “recombinant gene” is not a naturally-occurring gene. A recombinant gene is man-made. A recombinant gene includes a protein coding sequence operably linked to expression control sequences. Embodiments include, but are not limited to, an exogenous gene introduced into a microorganism, an endogenous protein coding sequence operably linked to a heterologous promoter (i.e., a promoter not naturally linked to the protein coding sequence) and a gene with a modified protein coding sequence (e.g., a protein coding sequence encoding an amino acid change or a protein coding sequence optimized for expression in the microorganism). The recombinant gene is maintained in the genome of the microorganism, on a plasmid in the microorganism or on a phage in the microorganism.

As used herein, the term “substantially homologous” or “substantially identical” in the context of two nucleic acids or polypeptides, generally refers to two or more sequences or subsequences that have at least 40%, 50%, 60%, 70%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

### II. Abbreviations

[symbol] denotes a reversible reaction.
→ denotes an irreversible reaction with a K_{eq} (equilibrium constant) value of more than 100.

Compounds:
AMP: adenosine monophosphate
GMP: guanosine monophosphate
HEPES: 4-(2-hydroxyethyl)-1-piperazineethanesulfonic acid/sodium salt
IMP: inosine monophosphate
MES: 2-(N-morpholino)ethanesulfonic acid/sodium salt
MOPS: 3-(N-morpholino)propanesulfonic acid/sodium salt
NA or Na: nicotinic acid or niacin
NAM: nicotinamide
NMP: nucleoside monophosphate or nucleotide
NR: nicotinamide riboside, including both reduced form (NRH) and oxidized form (NR⁺)
NMN: nicotinamide mononucleotide, including both reduced form (NMNH) and oxidized form (NMN⁺)
NAD: nicotinamide adenine dinucleotide, including both reduced form (NADH) and oxidized form (NAD)
NADP: nicotinamide adenine dinucleotide phosphate, including both reduced form (NADPH) and oxidized form (NADP⁺)
Pᵢ: inorganic orthophosphate ion
Poly(P)ₙ: polyphosphate with a degree of polymerization of n
PPᵢ: inorganic pyrophosphate, diphosphate
R1P: alpha-D-ribose-1-phosphate
R5P: alpha-D-ribose-5-phosphate, 5-O-phosphono-alpha-D-ribofuranose
PRPP: 5-phospho-alpha-D-ribose-1-diphosphate, 5-phosphoribosyl 1-pyrophosphate, phosphoribosylpyrophosphate, phosphoribosyl diphosphate
Tris: tris(hydroxymethyl)aminomethane

Enzymes:
6PGDH: 6-phosphogluconate dehydrogenase (EC 1.1.1.44)
6PGL: 6-phosphogluconolactonase (EC 3.1.1.31)
αGP: alpha-glucan phosphorylase (EC 2.4.1.1)
ADK: adenylate kinase (EC 2.7.4.3)
AdK: adenosine kinase (EC 2.7.1.20)
AK: acetate kinase (EC 2.7.2.1)
APRT: adenine phosphoribosyltransferase (EC 2.4.2.7)
CA: catalase (EC 1.11.1.6, EC 1.11.1.21, or EC 1.11.1.7)
CBP: cellobiose phosphorylase (EC 2.4.1.20)
CDP: cellodextrin phosphorylase (EC 2.4.1.49)
D-RI: D-ribose isomerase (EC 5.3.1.20)
D-XI D-xylose isomerase (EC 5.3.1.5)
DPP: diphosphatase (EC 3.6.1.1)
G6PDH: glucose-6-phosphate dehydrogenase (EC 1.1.1.49)
GDA: guanine deaminase (EC 3.5.4.3), nucleoside deaminase (cytosine/guanine deaminase)
GP: guanosine phosphorylase (EC 2.4.2.15)
HGPRT: hypoxanthine/guanine phosphoribosyltransferase (EC 2.4.2.8)
MPI: mannose 6-phosphate isomerase (EC 5.3.1.8)
IMPN: inosinate nucleosidase (EC 3.2.2.12)
NADK: NAD kinase (EC 2.7.1.23)
NAMPT: nicotinamide phosphoribosyltransferase (EC 2.4.2.12)
NMNAT: nicotinamide nucleotide adenylyltransferase (EC 2.7.7.1)
NaRK: nicotinate riboside kinase (EC 2.7.1.173)
NRK: nicotinamide riboside kinase (EC 2.7.1.22)
NRP: nicotinamide riboside phosphorylase (EC 2.4.1.4)
NO: nucleoside oxidase (NO, EC 1.1.3.39)
NOX: NADH oxidase (H₂O-forming) (EC 1.6.3.4), NAD(P)H oxidase (EC 1.6.3.2)
P3E: pentose 3-epimerase (EC 5.1.3.31)
PGM: phosphoglucomutase (EC 5.4.2.2)
PNP: purine nucleoside phosphorylase (EC 2.4.2.1)
PPGK: polyphosphate glucokinase (EC 2.7.1.63)
PPM: phosphopentomutase (EC 5.4.2.7)
PPMPN: pyrimidine/purine nucleotide 5′-monophosphate nucleosidase (EC 3.2.2.10)
PPK: polyphosphate kinase (PPK, EC 2.7.4.1)
PPT: polyphosphate: AMP phosphotransferase (EC 2.7.4.B2)
PyNP: pyrimidine-nucleoside phosphorylase (EC 2.4.2.2)
RP: ribose-5-phosphate isomerase (EC 5.3.1.6)
RuPE: D-xylulose 5-phosphate 3-epimerase (EC 5.1.3.1)
RK: ribokinase (EC 2.7.1.15)
SP: sucrose phosphorylase (EC 2.4.1.7)
TP: thymidine phosphorylase (EC 2.4.2.4)
UP: uridine phosphorylase (EC 2.4.2.3)
XDH: xanthine dehydrogenase (EC 1.17.1.4)
XK: polyphosphate xylulokinase (EC 2.7.1.17)
XO: xanthine oxidase (EC 1.17.3.2)

### III. NMN Synthesis Via α-D-Ribose-1-Phosphate as Intermediate

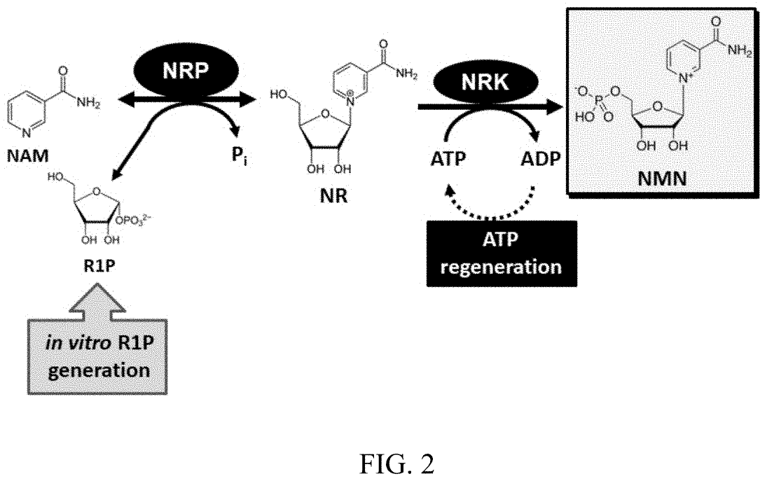

The current invention provides enzymatic reactions and methods to produce NMN via an intermediate α-D-ribose-1-phosphate and then nicotinamide riboside is converted to NMN catalyzed by nicotinamide riboside kinase (NRK, EC 2.4.1.22) (FIG. 2). Non-limiting examples of nicotinamide riboside kinase are listed in Table 1. Exemplary amino acid sequences of NRKs from Saccharomyces cerevisiae, Myceliophthora thermophila and Pseudomonas aeruginosa known in the art are respectively set out in SEQ ID NOs: 61-63. NRK may be replaced with promiscuous activity of some nicotinate riboside kinases (EC 2.7.1.173).

RIP Generation from Nucleosides

Inosine, guanosine, and adenosine are purine nucleosides comprising a respective purine base (hypoxanthine, guanine, and adenine) attached to a D-ribose ring via a β-N₉-glycosidic bond. They are cheap bulk biochemicals produced by industrial microbial fermentation. Inosine has a higher water solubility of 15.8-21 g/L at 20° C. than guanosine (0.7 g/L at 18° C.). Hypoxanthine has a low water solubility of 0.7 g/L at 20° C. while guanine is water insoluble but is soluble in diluted acid.

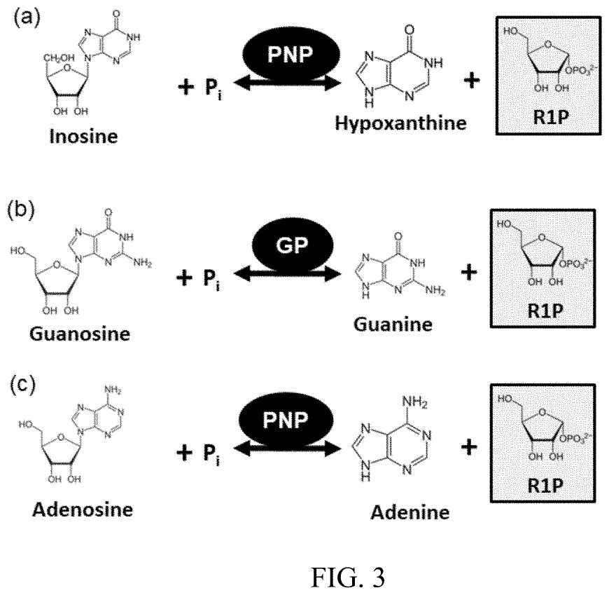

In one embodiment, alpha-D-ribose-1-phosphate (R1P) is generated from inosine, guanosine or adenosine catalyzed by purine nucleoside phosphorylase (PNP, EC 2.4.2.1) and/or guanosine nucleoside phosphorylase (GP, 2.4.2.15) (FIG. 3). Non-limiting examples of purine nucleoside phosphorylase (PNP) and/or guanosine nucleoside phosphorylase (GP, 2.4.2.15) are listed in Table 1. Exemplary amino acid sequences of PNPs from Bacillus halodurans, Thermus thermophilus, Thermotoga maritima MSB8, Meiothermus silvanus, Clostridium thermocellum, Geobacillus thermodenitrificans, and Deinococcus geothermalis known in the art are respectively set out in SEQ ID NOs: 29-35.

In one embodiment, alpha-D-ribose-1-phosphate (R1P) is generated from pyrimidine nucleosides and phosphate ions catalyzed by their respective pyrimidine-nucleoside phosphorylase (PyNP, EC 2.4.2.2), uridine phosphorylase (UP, EC 2.4.2.3) or thymidine phosphorylase (TP, EC 2.4.2.4).

RIP Generation from Nucleotides

Inosine monophosphate (IMP) is an ester of phosphoric acid with hypoxanthine. It is widely used as an umami flavor enhancer with E number (food additive) reference E630. It used to be made from chicken byproducts or other meat industry waste but it is produced by microbial fermentation. Guanosine monophosphate (GMP) is another flavor enhancer with E number reference E626. A blend of fermented products GMP, IMP and monosodium glutamate (MSG, E621) is widely used in Asian cuisine. Industrial production of both IMP and GMP was made either by RNA breakdown and nucleotide extraction and is produced by microbial fermentation using different microorganisms such as Corynebacterium, Bacillus, or Escherichia coli. Approximately 22,000 tons of GMP and IMP were produced in year 2010. Adenosine monophosphate (AMP) can be produced from RNA breakdown and nucleotide extraction and is produced by microbial fermentation using different microorganisms. The other NMPs (e.g., cytidine monophosphate, CMP; and uridine monophosphate, UMP) are not as cheap as IMP, GMP and AMP.

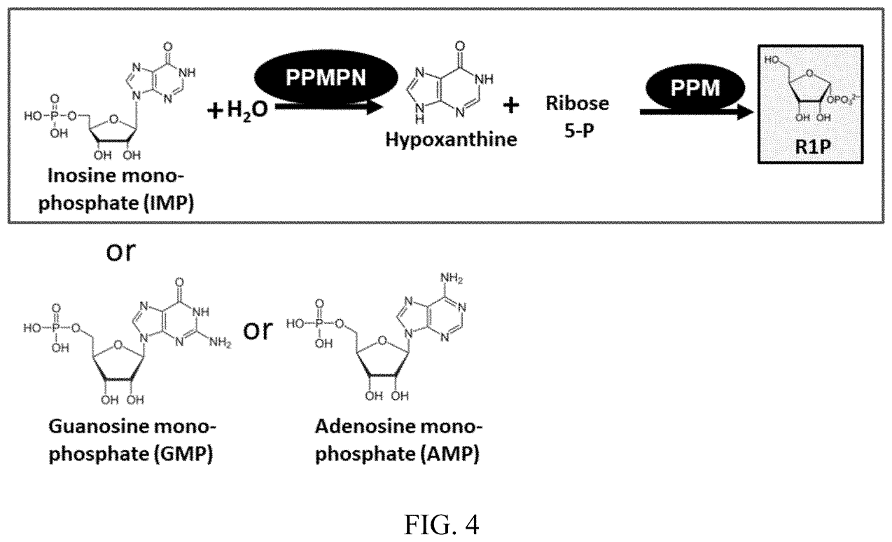

In one embodiment, alpha-D-ribose-1-phosphate is generated from a nucleotide inosine monophosphate (IMP) catalyzed by pyrimidine/purine nucleotide 5′-monophosphate nucleosidase (PPMPN, EC 3.2.2.10) or inosinate nucleosidase (EC 3.2.2.12) followed by phosphopentomutase (PPM, EC 5.4.2.7) (FIG. 4). Non-limiting examples of pyrimidine/purine nucleotide 5′-monophosphate nucleosidase and phosphopentomutase are listed in Table 1. Exemplary amino acid sequences of PPMPNs from Escherichia coli K-12, Shigella flexneri and Mannheimia succiniciproducens known in the art are respectively set out in SEQ ID NOs: 98-100. Exemplary amino acid sequences of PPMs from Thermotoga maritima, Thermus thermophilus and Clostridium thermocellum known in the art are respectively set out in SEQ ID NOs: 120-122.

In one embodiment, alpha-D-ribose-1-phosphate is generated from a nucleotide guanosine monophosphate (GMP) catalyzed by pyrimidine/purine nucleotide 5′-monophosphate nucleosidase (PPMPN, EC 3.2.2.10) and phosphopentomutase (PPM, EC 5.4.2.7) (FIG. 4). Exemplary amino acid sequences of PPMPNs from Escherichia coli K-12, Shigella flexneri and Mannheimia succiniciproducens known in the art are respectively set out in SEQ ID NOs: 98-100. Exemplary amino acid sequences of PPMs from Thermotoga maritima, Thermus thermophilus and Clostridium thermocellum known in the art are respectively set out in SEQ ID NOs: 120-122.

In one embodiment, alpha-D-ribose-1-phosphate is generated from a nucleotide adenosine monophosphate (AMP) catalyzed by pyrimidine/purine nucleotide 5′-monophosphate nucleosidase (PPMPN) or AMP nucleosidase (EC 3.2.2.4) and phosphopentomutase (PPM, EC 5.4.2.7) (FIG. 4). Exemplary amino acid sequences of PPMPNs from Escherichia coli K-12, Shigella flexneri and Mannheimia succiniciproducens known in the art are respectively set out in SEQ ID NOs: 98-100. Exemplary amino acid sequences of PPMs from Thermotoga maritima, Thermus thermophilus and Clostridium thermocellum known in the art are respectively set out in SEQ ID NOs: 120-122.

RIP Generation from D-Pentoses

D-ribose is an affordable pentose, which may be produced from glucose by microbial fermentation or converted from D-xylose catalyzed by three cascade enzymes: D-xylose isomerase (D-XI, EC 5.3.1.5), pentose 3-epimerase (P3E, EC 5.1.3.31), and D-ribose isomerase (D-RI, EC 5.3.1.20) or mannose 6-phosphate isomerase (MPI, EC 5.3.1.8).

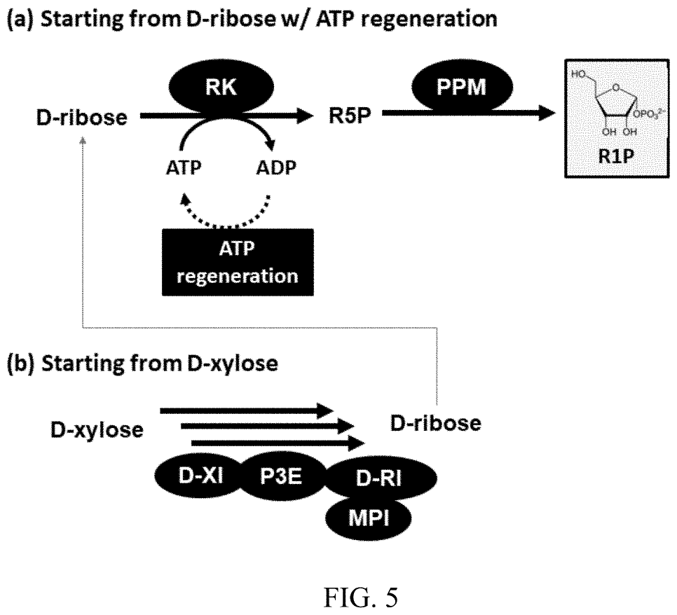

In one embodiment, alpha-D-ribose-1-phosphate is generated from D-ribose and ATP catalyzed by ribokinase (RK, EC 2.7.1.15) and phosphopentomutase (PPM, EC 5.4.2.7) (FIG. 5 step a). Non-limiting examples of ribokinase and phosphopentomutase are listed in Table 1. Exemplary amino acid sequences of RKs from Escherichia coli K12, Thermoanaerobacterium thermosaccharolyticum, and Thermus thermophilus known in the art are respectively set out in SEQ ID NOs: 53-55. Exemplary amino acid sequences of PPMs from Thermotoga maritima, Thermus thermophilus and Clostridium thermocellum known in the art are respectively set out in SEQ ID NOs: 120-122.

D-xylose is the most abundant pentose in nature. It is produced from hydrolysis of nonfood hemicellulose. It is called wood sugar and its prices were as low as sucrose in 1940s.

In one embodiment, alpha-D-ribose-1-phosphate is generated from D-xylose, which can be converted to D-ribose by D-xylose isomerase (D-XI, EC 5.3.1.5), pentose 3-epimerase (P3E, EC 5.1.3.31), and D-ribose isomerase (D-RI, EC 5.3.1.20) or mannose 6-phosphate isomerase (MPI, EC 5.3.1.8) (FIG. 5 step b), followed by two-enzyme biocatalysis by RK and PPM. Non-limiting examples of D-XI, P3E, D-RI, and MPI are listed in Table 1. Exemplary amino acid sequences of D-XIs from Thermus thermophilus HB8, Thermotoga neapolitana and Geobacillus stearothermophilus known in the art are respectively set out in SEQ ID NOs: 111-113. Exemplary amino acid sequences of P3Es from Pseudomonas cichorii and Rhodobacter sphaeroides known in the art are respectively set out in SEQ ID NOs: 109-110. Exemplary amino acid sequence of D-RI from Mycobacterium smegmatis known in the art is respectively set out in SEQ ID NO: 117. Exemplary amino acid sequence of MPI from Geobacillus thermodenitrificans known in the art is respectively set out in SEQ ID NO: 116.

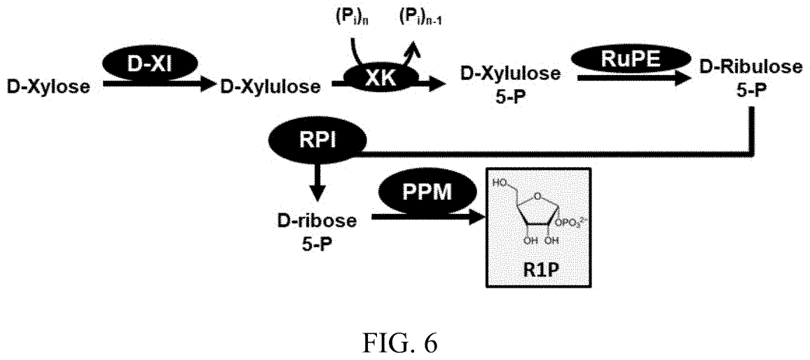

In one embodiment, alpha-D-ribose-1-phosphate is generated from D-xylose and polyphosphate (FIG. 6), whereas the artificial enzymatic pathway is comprised of D-xylose isomerase (D-XI, EC 5.3.1.5), polyphosphate xylulokinase (XK, EC 2.7.1.17), D-xylulose 5-phosphate 3-epimerase (RuPE, EC 5.1.3.1), ribose-5-phosphate isomerase (RPI, EC 5.3.1.6), and phosphopentomutase (PPM, EC 5.4.2.7). Non-limiting examples of D-XI, XK, RuPE, RPI, and PPM are listed in Table 1. Exemplary amino acid sequences of D-XIs from Thermus thermophilus HB8, Thermotoga neapolitana and Geobacillus stearothermophilus known in the art are respectively set out in SEQ ID NOs: 111-113. Exemplary amino acid sequences of polyphosphate XKs from Thermotoga maritima and Geobacillus stearothermophilus known in the art are respectively set out in SEQ ID NOs: 56-57. Exemplary amino acid sequences of RuPEs from Clostridium thermocellum and Thermotoga maritima known in the art are respectively set out in SEQ ID NOs: 107-108. Exemplary amino acid sequences of RPIs from Clostridium thermocellum and Thermotoga maritima known in the art are respectively set out in SEQ ID NOs: 114-115. Exemplary amino acid sequences of PPMs from Thermotoga maritima, Thermus thermophilus and Clostridium thermocellum known in the art are respectively set out in SEQ ID NOs: 120-122.

RIP Generation from Hexoses

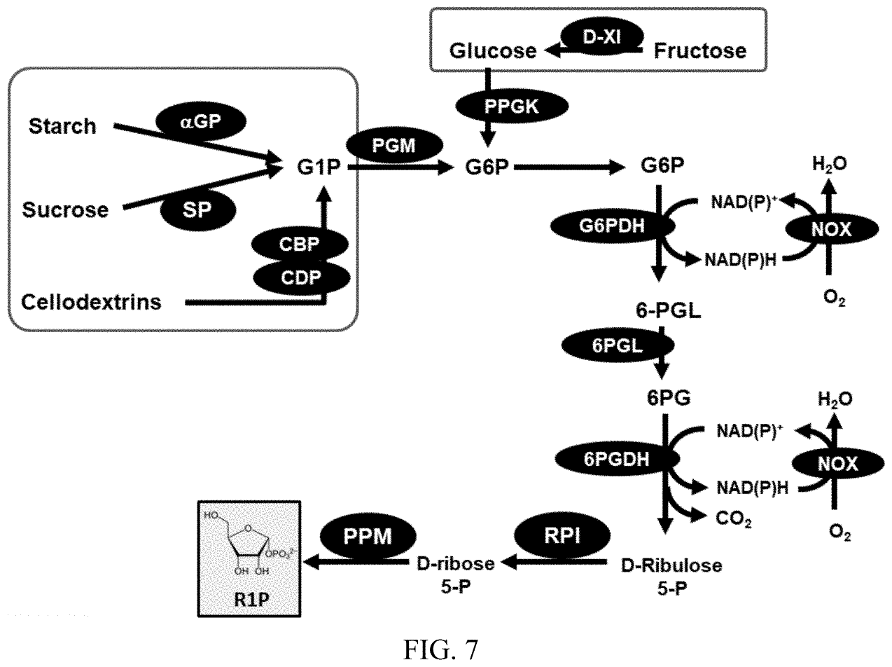

Hexoses are among the least costly natural sugars. Low-cost and representative hexoses can be classified into monosaccharides (e.g., D-glucose and D-fructose), oligosaccharides (e.g., sucrose, cellobiose, cellodextrins, maltose, maltodextrin, and amylodextrin), and polysaccharides (e.g., cellulose, starch and soluble starch). A few oligosaccharides and starch can be converted to glucose 1-phosphate via their respective phosphorylases without ATP. The examples of glucans and their glucan phosphorylases are starch, maltodextrin and amylopectin catalyzed by alpha-glucan phosphorylase (αGP, EC 2.4.1.1); cellodextrin and cellobiose catalyzed by cellodextrin phosphorylase (CDP, EC 2.4.1.49) and cellobiose phosphorylase (CBP, EC 2.4.1.20); sucrose phosphorylase (SP, EC 2.4.1.7), and cellulose catalyzed by engineered cellulose phosphorylase. Then glucose 1-phosphate is converted glucose 6-phosphate and then it is converted to ribose 5-phosphate via a partial pentose phosphate pathway that can convert one glucose 6-phosphate to one ribose 5-phosphate, two reduced NAD(P)H, and one CO₂ (FIG. 7). Then ribose 5-phosphate is converted to alpha-D-ribose-1-phosphate catalyzed by phosphopentomutase (PPM). For two most abundant monomer hexoses (i.e., glucose and fructose), they are converted to glucose 6-phosphate without ATP. Glucose is converted to glucose 6-phosphate with polyphosphate catalyzed by polyphosphate glucokinase (PPGK, EC 2.7.1.63). To utilize fructose, it is converted to glucose by xylose isomerase (D-XI, EC 5.3.1.5) followed by polyphosphate glucokinase. For water-insoluble cellulose, it is hydrolyzed to water soluble cellodextrin and cellobiose by beta-glucosidase-free cellulase mixture or partial acid hydrolysis.

In one embodiment, alpha-D-ribose-1-phosphate is generated from starch, maltodextrin, or amylodextrin and phosphate (FIG. 7), whereas the artificial enzymatic pathway is comprised of αGP (alpha-glucan phosphorylase, EC 2.4.1.1), PGM (phosphoglucomutase, EC 5.4.2.2), G6PDH (glucose-6-phosphate dehydrogenase, EC 1.1.1.49), 6-phosphogluconolactonase (6PGL, EC 3.1.1.31), 6PGDH (6-phosphogluconate dehydrogenase, EC 1.1.1.44), RPI (ribose 5-phosphate isomerase, EC 5.3.1.6), PPM (phosphopentomutase, EC 5.4.2.7), and H₂O-forming NAD(P)H oxidase (NOX, EC 1.6.3.4 or EC 1.6.3.2). Non-limiting examples of αGP, PGM, G6PDH, 6PGL, 6PGDH, RPI, PPM and NOX are listed in Table 1. Exemplary amino acid sequences of αGPs from Clostridium thermocellum, Thermotoga neapolitana and Thermococcus kodakarensis KOD1 known in the art are respectively set out in SEQ ID NOs: 16-18. Exemplary amino acid sequences of PGMs from Clostridium thermocellum and Thermus thermophilus known in the art are respectively set out in SEQ ID NOs: 118-119. Exemplary amino acid sequences of G6PDHs from Thermotoga maritima and Zymomonas mobilis known in the art are respectively set out in SEQ ID NOs: 3-4. Exemplary amino acid sequences of 6PGL from Thermotoga maritima known in the art are respectively set out in SEQ ID NOs: 94. Exemplary amino acid sequences of 6PGDHs from Thermotoga maritima and Moorella thermoacetica known in the art are respectively set out in SEQ ID NOs: 1-2. Exemplary amino acid sequences of RPIs from Clostridium thermocellum and Thermotoga maritima known in the art are respectively set out in SEQ ID NOs: 114-115. Exemplary amino acid sequences of PPMs from Thermotoga maritima, Thermus thermophilus and Clostridium thermocellum known in the art are respectively set out in SEQ ID NOs: 120-122. Exemplary amino acid sequences of NOXs from Clostridium aminovalericum, Clostridium acetobutylicum and Streptococcus mutans known in the art are respectively set out in SEQ ID NOs: 5-7.

In another embodiment, soluble starch or maltodextrin is debranched to linear amylodextrin by pullulanase (EC 3.2.1.41) and/or isoamylase (EC 3.2.1.68).

In another embodiment, maltose and maltotriose is converted to long-chain amylodextrin catalyzed by 4-alpha-glucanotransferase (EC 2.4.1.25).

In one embodiment, alpha-D-ribose-1-phosphate is generated from cellodextrin/cellobiose and phosphate (FIG. 7), whereas the artificial enzymatic pathway is comprised of CDP (cellodextrin phosphorylase, EC 2.4.1.49), CBP (cellobiose phosphorylase, EC 2.4.1.20), PGM, G6PDH, 6PGL, 6PGDH, RPI, PPM, and NOX. Non-limiting examples of CDP, CBP, PGM, G6PDH, 6PGL, 6PGDH, RPI, PPM and NOX are listed in Table 1. Exemplary amino acid sequences of CDPs from Clostridium thermocellum and Thermosipho africanus known in the art are respectively set out in SEQ ID NOs: 23-24. Exemplary amino acid sequences of CBPs from Clostridium thermocellum and Thermosipho africanus known in the art are respectively set out in SEQ ID NOs: 22 and 24. Exemplary amino acid sequences of PGMs from Clostridium thermocellum and Thermus thermophilus known in the art are respectively set out in SEQ ID NOs: 118-119. Exemplary amino acid sequences of G6PDHs from Thermotoga maritima and Zymomonas mobilis known in the art are respectively set out in SEQ ID NOs: 3-4. Exemplary amino acid sequences of 6PGL from Thermotoga maritima known in the art are respectively set out in SEQ ID NOs: 94. Exemplary amino acid sequences of 6PGDHs from Thermotoga maritima and Moorella thermoacetica known in the art are respectively set out in SEQ ID NOs: 1-2. Exemplary amino acid sequences of RPIs from Clostridium thermocellum and Thermotoga maritima known in the art are respectively set out in SEQ ID NOs: 114-115. Exemplary amino acid sequences of PPMs from Thermotoga maritima, Thermus thermophilus and Clostridium thermocellum known in the art are respectively set out in SEQ ID NOs: 120-122. Exemplary amino acid sequences of NOXs from Clostridium aminovalericum, Clostridium acetobutylicum and Streptococcus mutans known in the art are respectively set out in SEQ ID NOs: 5-7.

In another embodiment, water soluble cellodextrin and cellobiose is made through the enzymatic hydrolysis of cellulose catalyzed by that beta-glucosidase-free cellulase mixture containing endo-glucanase (EC 3.2.1.4) and cellobiohydrolase (EC 3.2.1.91).

In another embodiment, water soluble cellodextrin and cellobiose is made through the acidic hydrolysis of cellulose, wherein the strong acid may be fuming HCl, a mixture of concentrated HCl and concentrated sulfuric acid, a mixture of concentrated HCl and concentrated phosphoric acid, and/or their mixture, and so on.

In one embodiment, alpha-D-ribose-1-phosphate is generated from sucrose and phosphate (FIG. 7), whereas the artificial enzymatic pathway is comprised of SP (sucrose phosphorylase, EC 2.4.1.7), PGM, G6PDH, 6PGL, 6PGDH, RPI, PPM, and NOX. Non-limiting examples of SP, CBP, PGM, G6PDH, 6PGL, 6PGDH, RPI, PPM and NOX are listed in Table 1. Exemplary amino acid sequences of SPs from Bifidobacterium adolescentis, Leuconostoc mesenteroides and Thermoanaerobacterium thermosaccharolyticum known in the art are respectively set out in SEQ ID NOs: 19-21. Exemplary amino acid sequences of PGMs from Clostridium thermocellum and Thermus thermophilus known in the art are respectively set out in SEQ ID NOs: 118-119. Exemplary amino acid sequences of G6PDHs from Thermotoga maritima and Zymomonas mobilis known in the art are respectively set out in SEQ ID NOs: 3-4. Exemplary amino acid sequences of 6PGL from Thermotoga maritima known in the art are respectively set out in SEQ ID NOs: 94. Exemplary amino acid sequences of 6PGDHs from Thermotoga maritima and Moorella thermoacetica known in the art are respectively set out in SEQ ID NOs: 1-2. Exemplary amino acid sequences of RPIs from Clostridium thermocellum and Thermotoga maritima known in the art are respectively set out in SEQ ID NOs: 114-115. Exemplary amino acid sequences of PPMs from Thermotoga maritima, Thermus thermophilus and Clostridium thermocellum known in the art are respectively set out in SEQ ID NOs: 120-122. Exemplary amino acid sequences of NOXs from Clostridium aminovalericum, Clostridium acetobutylicum and Streptococcus mutans known in the art are respectively set out in SEQ ID NOs: 5-7.

In one embodiment, alpha-D-ribose-1-phosphate is generated from glucose and polyphosphate (FIG. 7), whereas the artificial enzymatic pathway is comprised of PPGK (polyphosphate glucokinase, EC 2.7.1.63), G6PDH, 6PGL, 6PGDH, RPI, PPM, and NOX. Non-limiting examples of PPGK, CBP, PGM, G6PDH, 6PGL, 6PGDH, RPI, PPM and NOX are listed in Table 1. Exemplary amino acid sequences of PPGK from Thermobifida fusca known in the art is set out in SEQ ID NO: 70. Exemplary amino acid sequences of PGMs from Clostridium thermocellum and Thermus thermophilus known in the art are respectively set out in SEQ ID NOs: 118-119. Exemplary amino acid sequences of G6PDHs from Thermotoga maritima and Zymomonas mobilis known in the art are respectively set out in SEQ ID NOs: 3-4. Exemplary amino acid sequences of 6PGL from Thermotoga maritima known in the art are respectively set out in SEQ ID NOs: 94. Exemplary amino acid sequences of 6PGDHs from Thermotoga maritima and Moorella thermoacetica known in the art are respectively set out in SEQ ID NOs: 1-2. Exemplary amino acid sequences of RPIs from Clostridium thermocellum and Thermotoga maritima known in the art are respectively set out in SEQ ID NOs: 114-115. Exemplary amino acid sequences of PPMs from Thermotoga maritima, Thermus thermophilus and Clostridium thermocellum known in the art are respectively set out in SEQ ID NOs: 120-122. Exemplary amino acid sequences of NOXs from Clostridium aminovalericum, Clostridium acetobutylicum and Streptococcus mutans known in the art are respectively set out in SEQ ID NOs: 5-7.

In one embodiment, alpha-D-ribose-1-phosphate is generated from fructose and polyphosphate (FIG. 7), whereas the artificial enzymatic pathway is comprised of D-XI (xylose isomerase, EC 5.3.1.5), PPGK, G6PDH, 6PGL, 6PGDH, RPI, PPM, and NOX. Non-limiting examples of D-XI, PPGK, CBP, PGM, G6PDH, 6PGL, 6PGDH, RPI, PPM and NOX are listed in Table 1. Exemplary amino acid sequences of D-XIs from Thermus thermophilus HB8, Thermotoga neapolitana and Geobacillus stearothermophilus known in the art are respectively set out in SEQ ID NOs: 111-113. Exemplary amino acid sequences of PPGK from Thermobifida fusca known in the art is set out in SEQ ID NO: 70. Exemplary amino acid sequences of PGMs from Clostridium thermocellum and Thermus thermophilus known in the art are respectively set out in SEQ ID NOs: 118-119. Exemplary amino acid sequences of G6PDHs from Thermotoga maritima and Zymomonas mobilis known in the art are respectively set out in SEQ ID NOs: 3-4. Exemplary amino acid sequences of 6PGL from Thermotoga maritima known in the art are respectively set out in SEQ ID NOs: 94. Exemplary amino acid sequences of 6PGDHs from Thermotoga maritima and Moorella thermoacetica known in the art are respectively set out in SEQ ID NOs: 1-2. Exemplary amino acid sequences of RPIs from Clostridium thermocellum and Thermotoga maritima known in the art are respectively set out in SEQ ID NOs: 114-115. Exemplary amino acid sequences of PPMs from Thermotoga maritima, Thermus thermophilus and Clostridium thermocellum known in the art are respectively set out in SEQ ID NOs: 120-122. Exemplary amino acid sequences of NOXs from Clostridium aminovalericum, Clostridium acetobutylicum and Streptococcus mutans known in the art are respectively set out in SEQ ID NOs: 5-7.

In one embodiment, alpha-D-ribose-1-phosphate is generated from a mixture of hexoses (e.g. starch, sucrose, glucose, fructose) and phosphate/or polyphosphate, with a mixture of the above enzymes (FIG. 7).

### IV. ATP Regeneration Systems

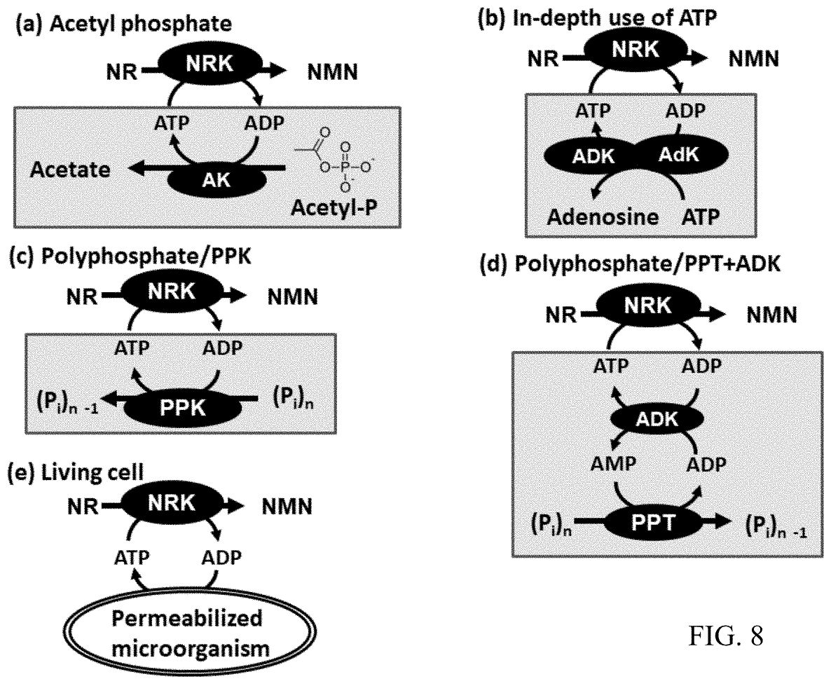

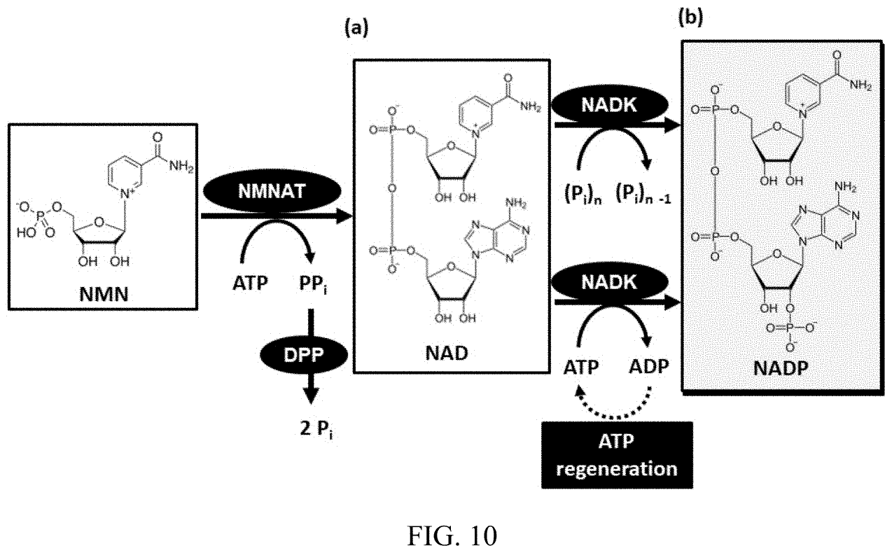

The current invention disclosure presents several commonly-used ATP regeneration (FIG. 8). These ATP regeneration methods can be used to produce NMN from NR (FIG. 2), produce NADP from NAD (FIG. 10 step b), and activate D-ribose to produce D-ribose 5-phosphate (FIG. 5 step a).

In one embodiment, ATP is regenerated from acetyl phosphate catalyzed by acetate kinase (AK, EC 2.7.2.1) (FIG. 8 step a), where the ATP consumption example was the synthesis of NMN from NR catalyzed by NR kinase (NRK, EC 2.7.1.22). ATP consumption example is not limited to the reaction catalyzed by NRK. Non-limiting examples of acetate kinase (AK, EC 2.7.2.1) are listed in Table 1. Exemplary amino acid sequences of AKs from Geobacillus stearothermophilus, Methanosarcina thermophila and Thermotoga maritima known in the art are respectively set out in SEQ ID NOs: 71-73.

In one embodiment, ATP is recycled for in-depth use by using adenylate kinase (ADK, EC 2.7.4.3) and adenosine kinase (AdK, EC 2.7.1.20) (FIG. 8 step b), where the ATP consumption example was the synthesis of NMN from NR catalyzed by NR kinase (NRK, EC 2.7.1.22). ATP consumption example is not limited to the reaction catalyzed by NRK. Non-limiting examples of ADK and AdK are listed in Table 1. Exemplary amino acid sequences of ADKs from Clostridium thermocellum, Sulfolobus acidocaldarius and Thermotoga maritima known in the art are respectively set out in SEQ ID NOs: 85-87. Exemplary amino acid sequences of AdKs from Myceliophthora thermophila, Thermothielavioides terrestris and Saccharomyces cerevisiae known in the art are respectively set out in SEQ ID NOs: 58-60.

In one embodiment, adenosine is hydrolyzed to adenine and ribose catalyzed by adenosine nucleosidase (AN, EC 3.2.2.7) or purine nucleosidase (PN, EC 3.2.2.1). Non-limiting examples of adenosine nucleosidase (AN, EC 3.2.2.7) or purine nucleosidase (PN, EC 3.2.2.1) are listed in Table 1. Exemplary amino acid sequences of ANs from Trypanosoma brucei brucei, Bacillus thuringiensis, and Escherichia coli K-12 known in the art are respectively set out in SEQ ID NOs: 95-97. Also, a typical AN can be isolated from Coffee arabica.

In one embodiment, ATP is regenerated from polyphosphate by using polyphosphate kinase (PPK, EC 2.7.4.1) (FIG. 8 step c), where the ATP consumption example was the synthesis of NMN from NR catalyzed by NR kinase (NRK, EC 2.7.1.22). ATP consumption example is not limited to the reaction catalyzed by NRK. Non-limiting examples of polyphosphate kinase (PPK, EC 2.7.4.1) are listed in Table 1. Exemplary amino acid sequences of PPKs from Corynebacterium glutamicum ATCC 13032, Cytophaga hutchinsonii, Deinococcus radiodurans, Meiothermus ruber, Deinococcus geothermalis, Mycobacterium tuberculosis, Pseudomonas aeruginosa ATCC 15692, and Thermus thermophilus HB27 known in the art are respectively set out in SEQ ID NOs: 74-82.

In one embodiment, ATP is regenerated from polyphosphate by using polyphosphate: AMP phosphotransferase (PPT, EC 2.7.4.B2) and adenylate kinase (ADK, EC 2.7.4.3) (FIG. 8 step d), where the ATP consumption example was the synthesis of NMN from NR catalyzed by NR kinase (NRK, EC 2.7.1.22). ATP consumption example is not limited to the reaction catalyzed by NRK. Non-limiting examples of polyphosphate: AMP phosphotransferase (PPT, EC 2.7.4.B2) and adenylate kinase (ADK, EC 2.7.4.3) are listed in Table 1. Exemplary amino acid sequences of PPTs from Acinetobacter johnsonii 210A and Myxococcus xanthus known in the art are respectively set out in SEQ ID NOs: 83-84. Exemplary amino acid sequences of ADKs from Clostridium thermocellum, Sulfolobus acidocaldarius, and Thermotoga maritima known in the art are respectively set out in SEQ ID NOs: 85-87.

In one embodiment, ATP is regenerated by using permeabilized cells (FIG. 8 step e), where the ATP consumption example was the synthesis of NMN from NR catalyzed by NR kinase (NRK, EC 2.7.1.22). ATP consumption example is not limited to the reaction catalyzed by NRK. The living cells (e.g., Escherichia coli, Saccharomyces cerevisiae) that is treated by an organic solvent can make ATP continuously.

In one embodiment, ATP is regenerated by using artificial or natural organelles (e.g., light-driven chloroplasts and mitochondria) that can make ATP continuously.

### V. NMN Synthesis with 5-Phospho-Alpha-D-Ribose-1-Diphosphate (PRPP) as Intermediate

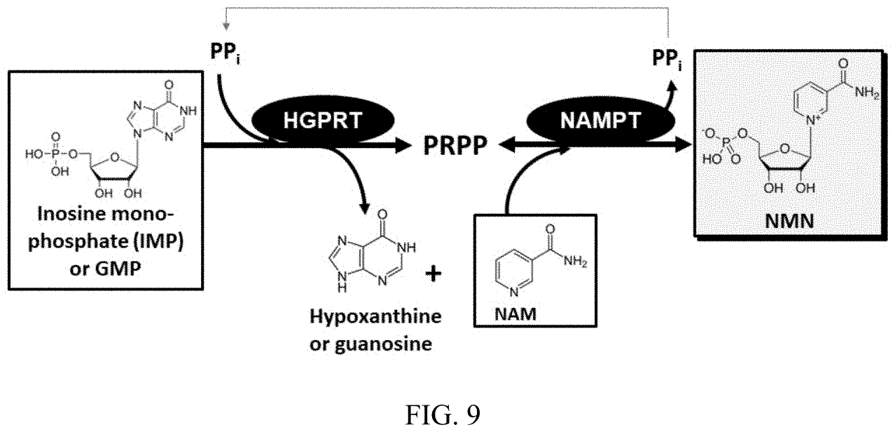

The current invention provides enzymatic reactions and methods to produce nicotinamide mononucleotide via an intermediate 5-phospho-α-D-ribose-1-diphosphate (PRPP). NMN can be synthesized from inosine monophosphate (IMP) or guanosine monophosphate (GMP) and nicotinamide catalyzed by two enzymes: hypoxanthine/guanine phosphoribosyltransferase (HGPRT, EC 2.4.2.8) and nicotinamide phosphoribosyltransferase (NAMPT, EC 2.4.2.12) (FIG. 9). The consolidation of these two reactions in one pot recycles both PRPP and PPᵢ.

In one embodiment, NMN can be synthesized from inosine monophosphate or guanosine monophosphate and nicotinamide catalyzed by two enzymes HGPRT (EC 2.4.2.8) and NAMPT (EC 2.4.2.12) in one pot. Non-limiting examples of HGPRTs and NAMPTs are listed in Table 1. Exemplary amino acid sequences of HGPRTs from Giardia intestinalis ATCC 50803, Trypanosoma cruzi, Gallus gallus (chick), Clostridium thermocellum, Haloferax volcanii and Halobacterium salinarum known in the art are respectively set out in SEQ ID NOs: 36-45. Exemplary amino acid sequences of NAMPTs from Haemophilus ducreyi, Shewanella oneidensis, Homo sapiens, Meiothermus ruber DSM 1279, Tenacibaculum maritimum, Marivirga tractuosa and Synechocystis sp. PCC 6803 known in the art are respectively set out in SEQ ID NOs: 46-52.

### VI. Synthesis of NMN Derivatives

NMN derivatives include nicotinamide adenine dinucleotide (NAD), nicotinamide adenine dinucleotide phosphate (NADP), and nicotinamide riboside (NR). NAD is reversibly synthesized from NMN catalyzed by nicotinamide nucleotide adenylyltransferase (NMNAT, EC 2.7.7.1). To increase NAD yield, the byproduct diphosphate is removed by diphosphatase (DPP, EC 3.6.1.1). NADP is reversibly synthesized from NAD and ATP or polyphosphate. To increase NADP yield, it is important to remove the byproduct. NR is synthesized from NMN catalyzed by 5′-nucleotidase (EC 3.1.3.5), nucleotide phosphatase (EC 3.1.3.31), or acid phosphatase (EC 3.1.3.2). This reaction is unidirectional so that the removal of other byproduct is not important to increase its yield.

In one embodiment, NAD is synthesized from NMN and ATP catalyzed by nicotinamide nucleotide adenylyltransferase (NMNAT, EC 2.7.7.1) (FIG. 10 step a). Non-limiting examples of NMNATs are listed in Table 1. Exemplary amino acid sequences of NMNATs from Methanocaldococcus jannaschii, Saccharomyces cerevisiae and Leishmania braziliensis known in the art are respectively set out in SEQ ID NOs: 88-90.

In one embodiment, NAD is synthesized from NR and ATP catalyzed by a bifunctional enzyme having activities of nicotinamide riboside kinase (NRK, EC 2.7.1.22) and nicotinamide nucleotide adenylyltransferase (NMNAT, EC 2.7.7.1). Non-limiting examples of these bifunctional enzymes (NRK/NMNATs) are listed in Table 1. Exemplary amino acid sequences of NRK/NMNATs from Clostridium thermocellum, Escherichia coli (strain K12) and Salmonella typhimurium known in the art are respectively set out in SEQ ID NOs: 91-93.

In one embodiment, diphosphate is hydrolyzed to two phosphate ions by diphosphatase (DPP, EC 3.6.1.1) (FIG. 10 step a). Non-limiting examples of DPPs are listed in Table 1. Exemplary amino acid sequences of DPPs from Thermoplasma acidophilum and Pyrococcus furiosus known in the art are respectively set out in SEQ ID NOs: 105-106.

In one embodiment, NADP is synthesized from NAD and ATP or polyphosphate catalyzed by ATP-dependent or polyphosphate-dependent NAD kinase (NADK, EC 2.7.1.23) (FIG. 10 step b). Non-limiting examples of NADKs are listed in Table 1. Exemplary amino acid sequences of NADKs from Clostridium thermocellum, Thermotoga maritima, Thermococcus kodakarensis, Pyrococcus horikoshii, Thermoanaerobacterium saccharolyticum and Microroccus luteus known in the art are respectively set out in SEQ ID NOs: 64-69.

In one embodiment, NADP is synthesized from NMN and ATP/polyphosphate catalyzed by NMNAT, DPP, and NADK (FIG. 10). Non-limiting examples of NMNATs, DPPs, and NADKs are listed in Table 1. Exemplary amino acid sequences of NMNATs from Methanocaldococcus jannaschii, Saccharomyces cerevisiae and Leishmania braziliensis known in the art are respectively set out in SEQ ID NOs: 88-90. Exemplary amino acid sequences of DPPs from Thermoplasma acidophilum and Pyrococcus furiosus known in the art are respectively set out in SEQ ID NOs: 105-106. Exemplary amino acid sequences of NADKs from Clostridium thermocellum, Thermotoga maritima, Thermococcus kodakarensis, Pyrococcus horikoshii, Thermoanaerobacterium saccharolyticum and Microroccus luteus known in the art are respectively set out in SEQ ID NOs: 64-69.

In one embodiment, NR is hydrolyzed from NMN, releasing inorganic phosphate (Pᵢ), catalyzed by 5′-nucleotidase (EC 3.1.3.5), nucleotide phosphatase (EC 3.1.3.31), acid phosphatase (EC 3.1.3.2), the same function enzyme, or their mixture.

### VII. Removal of Byproducts of Equilibrium Reactions

To shift the equilibrium reaction toward the formation of desired products, it is useful to in situ remove the byproducts of these equilibrium reactions by using the enzymes.

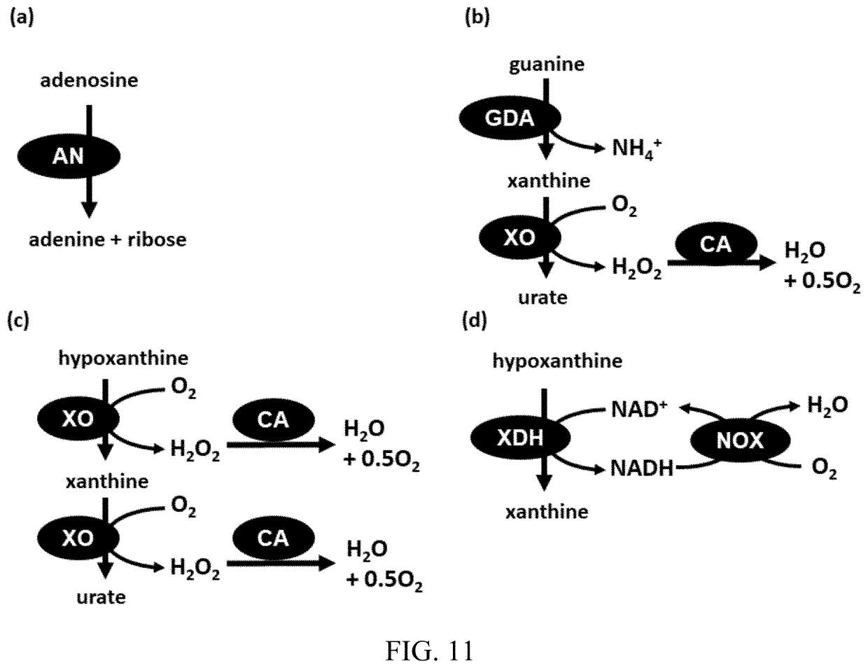

In one embodiment, adenosine is hydrolyzed to adenine and ribose by adenosine nucleosidase (AN, EC 3.2.2.7) or purine nucleosidase (PN, EC 3.2.2.1) (FIG. 11 step a). This hydrolysis reaction is unidirectional. Non-limiting examples of adenosine nucleosidase or purine nucleosidase are listed in Table 1. Exemplary amino acid sequences of ANs from Trypanosoma brucei brucei, Bacillus thuringiensis, and Escherichia coli K-12 known in the art are respectively set out in SEQ ID NOs: 95-97.

In one embodiment, guanine is converted to xanthine by guanine deaminase (GDA, EC 3.5.4.3) (FIG. 11 step b). Non-limiting examples of guanine deaminase are listed in Table 1. Exemplary amino acid sequences of GDAs from Bacillus subtilis 168, Haloferax volcanii, and Halobacterium salinarum known in the art are respectively set out in SEQ ID NOs: 101-104.

In one embodiment, xanthine is converted to urate by xanthine oxidase (XO, EC 1.17.3.2) or xanthan dehydrogenase (XDH, EC 1.17.3.4) followed by catalase (CA, EC 1.11.1.6, EC 1.11.1.21, or EC 1.11.1.7) (FIG. 11 step b). Non-limiting examples of xanthine oxidase, xanthan dehydrogenase and catalase are listed in Table 1. Exemplary amino acid sequences of XOs and XDHs from Bos taurus (bovine), Homo sapiens, Rattus norvegicus, Escherichia coli K-12 and Blastobotrys adeninivorans known in the art are respectively set out in SEQ ID NOs: 11-15. Exemplary amino acid sequences of CAs from Thermus brockianus, Bacillus pumilus and Geobacillus stearothermophilus known in the art are respectively set out in SEQ ID NOs: 8-10.

In one embodiment, hypoxanthine can be converted to xanthine and then to urate by xanthine oxidase (XO, EC 1.17.3.2) and catalase (FIG. 11 step c). Non-limiting examples of xanthine oxidase and catalase are listed in Table 1. Exemplary amino acid sequences of XOs from Bos taurus (bovine), Homo sapiens, Rattus norvegicus, Escherichia coli K-12 and Blastobotrys adeninivorans known in the art are respectively set out in SEQ ID NOs: 11-15. Exemplary amino acid sequences of CAs from Thermus brockianus, Bacillus pumilus and Geobacillus stearothermophilus known in the art are respectively set out in SEQ ID NOs: 8-10.

In one embodiment, hypoxanthine can be converted to xanthine by xanthine dehydrogenase (XDH, EC 1.17.1.4) and H₂O-forming NADH oxidase (NOX, EC 1.6.3.4 or EC 1.6.3.2) (FIG. 11 step d). Non-limiting examples of XDHs and NOXs are listed in Table 1. Exemplary amino acid sequences of XOs from Bos taurus (bovine), Homo sapiens, Rattus norvegicus, Escherichia coli K-12 and Blastobotrys adeninivorans known in the art are respectively set out in SEQ ID NOs: 11-15. Exemplary amino acid sequences of NOXs from Clostridium aminovalericum, Clostridium acetobutylicum and Streptococcus mutans known in the art are respectively set out in SEQ ID NOs: 5-7.

In one embodiment, NOX may be replaced with other NAD(P)H-consuming enzymes. For example, hydrogenase replaces a mixture of H₂O₂ formation NADH oxidase (EC 1.6.3.3) and catalase.

In one embodiment, diphosphate can be converted to two phosphate ions by (inorganic) diphosphatase (DPP, EC 3.6.1.1). The hydrolysis of diphosphate is unidirectional. Non-limiting examples of DPPs are listed in Table 1. Exemplary amino acid sequences of DPPs from Thermoplasma acidophilum and Pyrococcus furiosus known in the art are respectively set out in SEQ ID NOs: 105-106.

### VIII. Enzymes

Enzyme Variants

Enzymes disclosed in the invention are naturally occurring in various organisms. While specific enzymes with the desired activity are used in the examples, the invention is not limited to these enzymes as other enzymes may have similar activities and can be used. For example, nucleoside phosphorylases catalyze the reversible phosphorolysis of purines and pyrimidines. Specifically, purine nucleoside phosphorylase has been demonstrated to catalyze the similar reaction on nicotinamide riboside, although nicotinamide is neither a purine nor a pyrimidine. It may be discovered that some pyrimidine nucleoside phosphorylases may also catalyze this reaction and is disclosed in this invention. Other reactions described in this invention may be catalyzed by enzymes not described in the embodiment, and are also incorporated into the embodiment.

In certain embodiments, variants of these enzymes in which the catalytic activity has been modified, e.g., to make it more active and stable in acidic conditions, may be used in the invention. Amino acid sequence variants of the polypeptide include substitution, insertion, or deletion variants, and variants may be substantially homologous or substantially identical to the unmodified polypeptides.

In certain embodiments, the variants retain at least some of the biological activity, e.g., catalytic activity, of the polypeptide. Other variants include variants of the polypeptide that retain at least about 50%, preferably at least about 75%, more preferably at least about 90%, of the biological activity.

In certain embodiments, substitutional variants typically exchange one amino acid for another at one or more sites within the protein. Substitutions of this kind can be conservative, that is, one amino acid is replaced with one of similar shape and charge. Conservative substitutions include, for example, the changes of: alanine to serine; arginine to lysine; asparagine to glutamine; aspartate to glutamate; cysteine to serine; glutamine to asparagine; glutamate to aspartate; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. An example of the nomenclature used herein to indicate an amino acid substitution is “S345F ThrA” wherein the naturally occurring serine occurring at position 345 of the naturally occurring ThrA enzyme which has been substituted with a phenylalanine.

A polypeptide or polynucleotide “derived from” an organism contains one or more modifications to the naturally-occurring amino acid sequence or nucleotide sequence and exhibits similar, if not better, activity compared to the native enzyme (e.g., at least 70%, at least 80%, at least 90%, at least 95%, at least 100%, or at least 110% the level of activity of the native enzyme). For example, enzyme activity is improved in some contexts by directed evolution of a parent/naturally-occurring sequence. Additionally or alternatively, an enzyme coding sequence is mutated to achieve less product inhibition.

Forms of the Enzymes

The isolated enzymes used in this invention are water soluble. It is often preferable to use immobilized enzymes. Immobilized enzymes are often more stable and robust, including examples of purine nucleoside phosphorylases (Hori, N., Watanabe, M., Sunagawa, K., Uehara, K., Mikami, Y. (1991) Production of 5-methyluridine by immobilized thermostable purine nucleoside phosphorylase and pyrimidine nucleoside phosphorylase from Bacillus stearothermophilus JTS 859. Journal of Biotechnology 17: 121-131). Immobilized enzymes are also easier to recover and use in multiple catalytic cycles, thus lowering the cost of an industrial process. Multiple means of enzyme immobilization are known in the art, as summarized (Es, I., Vieira, J. D. G., Amaral, A. C., (2015) “Principles, techniques, and applications of biocatalyst immobilization for industrial application” Applied Microbiology and Biotechnology 99:2065-2082). Enzymes may also be crossed linked to form Cross Linked Enzyme Aggregates (CLEAs) (Schoevaart, R., Wolbers, M. W., Golubovic, M., Ottens, M., Kieboom, A. P. G., van Rantwijk, F., van der Wielen, L. A. M., Sheldon, R. A., (2004) “Preparation, optimization, and structures of cross-linked enzyme aggregates (CLEAs)” Biotechnology and Bioengineering 87 (6): 754-762) which are often more stable and are easier to recover and reuse. Many of these enzymes are found together in organisms which can be used as biocatalysts to produce nucleosides (Trelles, J. A., Fernindez-Lucas, J., Condezo, L. A., Sinisterra, J. V. (2004) “Nucleoside synthesis by immobilized bacterial whole cells” Journal of Molecular Catalysis B: Enzymatic 30: 219-227), but they may also be heterologously expressed in engineered microorganisms, which then can be used as biocatalysts. Methods of immobilization and crosslinking of enzymes catalyzing the reactions disclosed in this invention are incorporated herein. The cell lysates made from said enzyme-overexpressed whole cells, which may be treated by heat or selective filtration to remove other cellular components (e.g., other proteins, membrane), can be mixed to reconstitute the artificial enzymatic pathways to synthesize the desired products.

Several whole cells contained the one or several said enzymes that may be permeabilized by heat, enzymes, or an organic solvent can be reconstituted to make the artificial enzymatic pathways to synthesize the desired products.

The purified enzymes, immobilized enzymes, permeabilized whole cells containing the said enzymes, or cell lysates or whole-cells or permeabilized whole-cells, and mixtures thereof, can be mixed to make the artificial enzymatic pathways to synthesize the desired products.

One whole-cell contained the all said enzymes that may be permeabilized by heat, enzymes, or an organic solvent can be used to make the artificial enzymatic pathways to synthesize the desired products.

### IX. One-Pot Process of Producing NMN, NAD, and NADP

Process Optimization

The reactions disclosed herein can be performed simultaneously within one bioreactor or pot or vessel. The bioreactor can be operated in the exposure of air or in anoxic conditions. The anoxic conditions are preferred, whereas the deoxygenated aqueous pH-controlled buffer containing said metal ions is a preferred aqueous solvent. The experimental conditions are optimized to trade off their different optimal conditions. The conditions including, but not limited to temperature, pH, solvent, timing of addition of reactants, length of reaction, concentration, agitation and deoxygenation (by vacuum or heating and nitrogen or CO₂ flashing) may be optimized for the consolidated bioreaction in one pot.

Production in Engineered Organisms

Enzymes catalyzing some or all of the reactions described in this invention may be expressed in non-natural, engineered heterologous organisms for the production of desired products. Specifically, genes coding for the enzymatic pathway enzymes may be isolated, inserted into expression vectors used to transform a production organism, may be incorporated into the genome, and direct expression of the enzymes. Protocols used to manipulate organisms are known in the art and explained in publications, such as Current Protocols in Molecular Biology, Online ISBN: 9780471142720, John Wiley and Sons, Inc., and Microbial Metabolic Engineering: Methods and Protocols, Qiong Cheng Ed., Springer.

Those familiar to the art can culture the engineered microbial cells to convert feedstocks such as carbohydrates or other precursors into nicotinamide riboside, and recover the nicotinamide mononucleotide. Guidance and protocols can be found in publications such as Fermentation and Biochemical Engineering Handbook: Principles, Process Design, and Equipment, 2d Edition, Henry C. Vogel and Celeste L. Todaro, Noyes Publications 1997.

NMN, NAD, NADP, or nicotinamide riboside may be separated from enzymes and reactants, and recovered from the reaction medium using a variety of procedures known in the art. These include, but are not limited to, crystallization, adsorption and release from ionic, hydrophobic and size exclusion resins, filtration, microfiltration, extraction, precipitation as salts or with solvents, or combinations thereof. The extent of the separation required may be limited to the removal of the enzymes, and a mixture of some or all of the remaining products and reactants including nicotinamide riboside, nicotinic acid riboside, 1,4-dihydronicotinamide riboside, nicotinamide, nicotinic acid, 1,4-dihydronicotinamide, inorganic orthophosphate, inorganic diphosphate, α-D-ribose-1-phosphate, 5-phospho-α-D-ribose-1-diphosphate, D-ribose-5-phosphate, β-nicotinamide D-ribonucleotide, and nitrogenous bases may be useful without further purification. Recovery of nicotinamide mononucleotide, NAD, NADP and NR with or without other products and reactants is a further embodiment of the invention.

### X. Examples

Chemicals and materials. All chemicals were reagent grade or higher, purchased from Sigma-Aldrich (St. Louis, Mo., USA) or Sinopharm (Shanghai, China), unless otherwise noted. Genomic DNA samples of microorganisms, such as Clostridium thermocellum, Thermotoga maritima, Aquifex aeolicus and so on were purchased from the American Type Culture Collection (Manassas, Va., USA), DSMZ—German Collection of Microorganisms and Cell Cultures (Braunschweig, Germany), and China General Microbiological Culture Collection Center (CGMCC, Beijing, China), and Japan Collection of Microorganisms (Ibaraki, Japan).

Escherichia coli TOP10 and E. coli DH5alpha (Thermo Fisher Scientific, Maltham, Mass., USA) were used for DNA manipulation and plasmid amplification. E. coli BL21(DE3) (Invitrogen, Carlsbad, Calif., USA) was used for recombinant protein expression. The Luria-Bertani (LB) medium (Miller formula) supplemented with either 100 μg/L ampicillin or 50 μg/L kanamycin was used for E. coli cell cultures and recombinant protein expression. All enzymes for molecular biology experiments were purchased from New England Biolabs (NEB, Ipswich, Mass., USA), unless otherwise used. Super GelRed™ was used as DNA gel stain (US Everbright Inc., Suzhou, China).

### Example 1. HPLC Assay

Samples containing NAM and NR were injected directly in the Shimadzu HPLC equipped with a Princeton Chromatograph Inc. SPHER-60 AMINO (NH₂) column (250×4.6 mm) (Princeton Chromatograph Inc., Cranbury, N.J., USA). The mobile phase was 20 mM KH₂PO₄. NAM and NR were detected spectroscopically at 261 nm by a Waters 2487 detector (Waters, Milford, Mass., USA) and quantified by comparison to standards from Sigma-Aldrich.

### Example 2. HPLC Assay

The concentrations of NAM, NMN, NAD, and PRPP in sample can also be measured by HPLC equipped with a Supelco Supelcosil LC-18-T (C18) reversed-phase column (250×4.6 mm; Supelco, Bellefonte, Pa., USA) and a Waters 2487 detector for the absorbance at 261 nm and quantified by comparison to standards. The mobile phase was used by mixing Buffer 1 (0.1 M potassium phosphate buffer, pH 6.0) and Buffer 2 (buffer 1 containing 20% methanol).

### Example 3. Sample Preparation Before HPLC Assays and Enzymatic Assays

500 of the aqueous solution after the enzymatic reaction was quenched by adding 125 μl of 1 M HClO₄. After precipitation at 18,000 g, and 500 μL of the supernatant was neutralized with 40 μl of 3 M K₂CO₃. After centrifugation, the supernatants were used for HPLC assays and enzymatic assays.

### Example 4. NAD/NADH Colorimetric Assay Kit

The concentrations of NAD (including NAD⁺ and NADH) were measured by the BioVision Inc. NAD/NADH Quantitation Colorimetric Kit (Catalog K337) (BioVision Inc. Milpitas, Calif., USA). This kit used the NAD-specific dehydrogenase that reduced NAD to NADH. NADH reacts with a colorimetric probe that produces a colored product which can be measured at 450 nm. There was no interference of NADP, NMN and NR.

### Example 5. NADP/NADPH Colorimetric Assay Kit

The concentrations of NADP (including NADP⁺ and NADPH) were measured by the Cell Biolabs Inc. NADP/NADPH quantitation colorimetric Kit (Catalog MET-5018) (Cell Biolabs Inc., San Diego, Calif. 92126 USA). This kit used the NADP-specific dehydrogenase that reduced NADP⁺ to NADPH. NADPH reacts with a colorimetric probe that produces a colored product which can be measured at 450 nm. There was no interference of NAD, NMN and NR.

### Example 6. Plasmid Construction

All recombinant enzymes were over-expressed by E. coli BL21(DE3). The pET plasmids encoding corresponding enzymes whose amino acid sequences were referred in SEQ ID. 1-122 were prepared by prolonged overlap extension PCR (You, C., X.-Z. Zhang and Y.-H. P. Zhang (2012). “Simple Cloning: direct transformation of PCR product (DNA multimer) to Escherichia coli and Bacillus subtilis.” Applied and Environmental Microbiology. 78: 1593-1595). Some of their DNA sequences were amplified by PCR based on available genomic DNA templates, especially for bacteria microorganisms, such as Clostridium thermocellum, Bacillus subtilis, E. coli, T. maritima, and so on. The other DNA sequences were synthesized with codon optimization by some gene-synthesized companies, such as GenScript (Piscataway, N.J., USA) and Qinglan Biotech (Wuxi, Jiangsu, China)

### Example 7. Production and Purification of Recombinant His-Tagged Enzymes

All recombinant enzymes overexpressed by E. coli BL21(DE3) were purified based on their His-tags through affinity adsorption on nickel-charged resin, unless otherwise noted. E. coli BL21 cells harboring pET plasmid encoding a target protein were cultivated in 250 mL of the LB medium at 37° C. Protein expression was induced by adding isopropyl-β-d-thiogalacto-pyranoside (IPTG) to a final concentration of 0.1 mM until A₆₀₀ reached ˜0.6-0.8. Protein expression was cultivated at 37° C. for 6 h or 18° C. for 16 h. Cell pellets were harvested by centrifugation and then were re-suspended in 50 mM HEPES buffer (pH 7.5) containing 0.1 M NaCl and 10 mM imidazole. After sonication and centrifugation, the supernatant was loaded onto the column packed with nickel-charged resins. The purified protein was eluted with 50 mM HEPES buffer (pH 7.5) containing 0.1 M NaCl and 150-500 mM imidazole. Mass concentration of protein was measured by the Bradford assay with bovine serum albumin as the standard. Protein expression and purity of recombinant proteins were examined by SDS-PAGE and analyzed by using densitometry analysis of the Image Lab software (Bio-Rad, Hercules, Calif., USA).

### Example 8. Simple Purification for Thermophilic Enzymes

Some of recombinant enzymes that originated from the thermophilic microorganisms were partially purified by simple heat treatment (e.g., 50-80° C. for 10-30 min). After centrifugation, the supernatants of cell lysates containing the said enzymes were used to implement the enzyme-catalyzed biosynthesis in one pot.

### Example 9: Synthesis of NMN from NAM, R1P and ATP

One-pot biosynthesis of NMN was conducted at 37° C. in a 100 mM HEPES buffer (pH 7.0) containing 10 mM MgCl₂, 20 mM NAM, 10 mM R1P, and 10 mM ATP. The recombinant enzymes were 1 U/mL nicotinamide riboside phosphorylase (NRP) and 1 U/mL nicotinamide riboside kinase (NRK). An aliquot of mixed recombinant NRP (E.C. 2.4.2.1, SEQ ID NOs: 25-28) and NRK (EC 2.7.1.22, SEQ ID NOs: 61-63) was added to the solution to initiate the reaction. The biotransformation was incubated at 37° C. until significant amount of NMN was generated. NMN concentration was measured by both HPLC and enzymatic assays.

### Example 10: Synthesis of R1P from Inosine and Phosphate

The synthesis of R1P was carried out at 37° C. in a 100 mM HEPES buffer (pH 7.0) containing 10 mM MgCl₂, 50 mM inosine and 20 mM inorganic sodium phosphate. The recombinant enzyme was 1 U/mL purine nucleoside phosphorylase (PNP, EC 2.4.2.1, SEQ ID NOs: 29-35). An aliquot of PNP was added to the solution to initiate the reaction. The biotransformation was incubated at 37° C. until significant amount of R1P was generated. R1P concentration was measured by HPLC.

### Example 11: Synthesis of R1P from Guanosine and Phosphate

The synthesis of R1P was carried out at 37° C. in a 100 mM HEPES buffer (pH 7.0) containing 10 mM MgCl₂, 10 mM guanosine and 20 mM inorganic sodium phosphate. The recombinant enzyme was 1 U/mL purine nucleoside phosphorylase (PNP, EC 2.4.2.1) or guanosine phosphorylase (GP, EC 2.4.2.5, SEQ ID NOs: 29-35). An aliquot of PNP or GP was added to the solution to initiate the reaction. The biotransformation was incubated at 37° C. until significant amount of R1P was generated. R1P concentration was measured by HPLC.

### Example 12: Synthesis of R1P from Adenosine and Phosphate

The synthesis of R1P was carried out at 37° C. in a 100 mM HEPES buffer (pH 7.0) containing 10 mM MgCl₂, 50 mM adenosine and 20 mM inorganic sodium phosphate. The recombinant enzyme was 1 U/mL purine nucleoside phosphorylase (PNP, EC 2.4.2.1, SEQ ID NOs: 29-35). An aliquot of PNP was added to the solution to initiate the reaction. The biotransformation was incubated at 37° C. until significant amount of R1P was generated. R1P concentration was measured by HPLC.

### Example 13: Synthesis of R1P from Inosine Monophosphate

The synthesis of R1P was carried out at 37° C. in a 100 mM HEPES buffer (pH 7.0) containing 10 mM MgCl₂ and 20 mM inosine monophosphate. The recombinant enzymes were 1 U/mL pyrimidine/purine nucleotide 5′-monophosphate nucleosidase (PPMPN, EC 3.2.2.10, SEQ ID NOs: 98-100) and 1 U/mL phosphopentomutase (PPM, EC 5.4.2.7, SEQ ID NOs: 120-122). An aliquot of mixed PPMPN and PPM was added to the solution to initiate the reaction. The biotransformation was incubated at 37° C. until significant amount of R1P was generated. R1P concentration was measured by HPLC.

### Example 14: Synthesis of R1P from Guanosine Monophosphate

The synthesis of R1P was carried out at 37° C. in a 100 mM HEPES buffer (pH 7.0) containing 10 mM MgCl₂ and 20 mM guanosine monophosphate. The recombinant enzymes were 1 U/mL pyrimidine/purine nucleotide 5′-monophosphate nucleosidase (PPMPN, EC 3.2.2.10, SEQ ID NOs: 98-100) and 1 U/mL phosphopentomutase (PPM, EC 5.4.2.7, SEQ ID NOs: 120-122). An aliquot of mixed PPMPN and PPM was added to the solution to initiate the reaction. The biotransformation was incubated at 37° C. until significant amount of R1P was generated. R1P concentration was measured by HPLC.

### Example 15: Synthesis of R1P from Adenosine Monophosphate

The synthesis of R1P was carried out at 37° C. in a 100 mM HEPES buffer (pH 7.0) containing 10 mM MgCl₂ and 40 mM adenosine monophosphate. The recombinant enzymes were 1 U/mL pyrimidine/purine nucleotide 5′-monophosphate nucleosidase (PPMPN, EC 3.2.2.10, SEQ ID NOs: 98-100) and 1 U/mL phosphopentomutase (PPM, EC 5.4.2.7, SEQ ID NOs: 120-122). An aliquot of mixed PPMPN and PPM was added to the solution to initiate the reaction. The biotransformation was incubated at 37° C. until significant amount of R1P was generated. R1P concentration was measured by HPLC.

### Example 16: Synthesis of R1P from D-Ribose and ATP

The synthesis of R1P was carried out at 37° C. in a 100 mM HEPES buffer (pH 7.0) containing 10 mM MgCl₂, 20 mM ATP, and 50 mM ribose. The recombinant enzymes were 1 U/mL ribokinase (RK, EC 2.7.1.15, SEQ ID NOs: 53-55) and 1 U/mL phosphopentomutase (PPM, EC 5.4.2.7, SEQ ID NOs: 120-122). An aliquot of mixed RK and PPM was added to the solution to initiate the reaction. The biotransformation was incubated at 37° C. until significant amount of R1P was generated. R1P concentration was measured by HPLC.

### Example 17: Synthesis of R1P from D-Xylose and ATP

The synthesis of R1P was carried out at 37° C. in a 100 mM HEPES buffer (pH 7.0) containing 10 mM MgCl₂, 20 mM ATP, and 100 mM D-xylose. The recombinant enzymes were 1 U/mL D-xylose isomerase (D-XI, EC 5.3.1.5, SEQ ID NOs: 111-113), pentose 3-epimerase (P3E, EC 5.1.3.31, SEQ ID NOs: 109-110), and D-ribose isomerase (D-RI, EC 5.3.1.20, SEQ ID No: 117) or mannose 6-phosphate isomerase (MPI, EC 5.3.1.8, SEQ ID No: 116), and 1 U/mL phosphopentomutase (PPM, EC 5.4.2.7, SEQ ID Nos: 120-122). An aliquot of mixed enzymes was added to the solution to initiate the reaction. The biotransformation was incubated at 37° C. until significant amount of R1P was generated. R1P concentration was measured by HPLC.

### Example 18: Synthesis of R1P from D-Ribose and Polyphosphate

The synthesis of R1P was carried out at 37° C. in a 100 mM HEPES buffer (pH 7.0) containing 30 mM MgCl₂, 20 mM polyphosphate, and 50 mM ribose. The recombinant enzymes were 1 U/mL D-xylose isomerase (D-XI, EC 5.3.1.5, SEQ ID Nos: 111-113), 1 U/mL polyphosphate xylulokinase (XK, EC 2.7.1.17, SEQ ID Nos: 56-57), 1 U/mL D-xylulose 5-phosphate 3-epimerase (RuPE, EC 5.1.3.1, SEQ ID Nos: 107-108), 1 U/mL ribose-5-phosphate isomerase (RPI, EC 5.3.1.6, SEQ ID Nos: 114-115), and 1 U/mL phosphopentomutase (PPM, EC 5.4.2.7, SEQ ID Nos: 120-122). An aliquot of mixed enzymes was added to the solution to initiate the reaction. The biotransformation was incubated at 37° C. until significant amount of R1P was generated. R1P concentration was measured by HPLC.

### Example 18: Synthesis of R1P from Starch and Phosphate

The synthesis of R1P was carried out at 37° C. in a 100 mM HEPES buffer (pH 7.0) containing 10 mM MgCl₂, 10 mM phosphate, 5 mM NAD(P)+, and 20 mM starch (maltodextrin). The enzymes added (1 U/mL) were αGP (alpha-glucan phosphorylase, EC 2.4.1.1, SEQ ID Nos: 16-18), PGM (phosphoglucomutase, EC 5.4.2.2, SEQ ID Nos: 118-119), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 19: Synthesis of R1P from Starch and Phosphate

The synthesis of R1P was carried out at 37° C. in a 100 mM HEPES buffer (pH 7.0) containing 10 mM MgCl₂, 10 mM phosphate, 5 mM NAD(P)+, and 20 mM starch (maltodextrin). Starch or maltodextrin may be treated by isoamylase or pullulanase before its use. The enzymes added (1 U/mL, each) were αGP (alpha-glucan phosphorylase, EC 2.4.1.1, SEQ ID Nos: 16-18), PGM (phosphoglucomutase, EC 5.4.2.2, SEQ ID Nos: 118-119), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. RIP concentration was measured by HPLC. Debranched starch treated by isoamylase or pullulanase resulted in about 10-30% more R1P than non-treated starch.

### Example 20: Synthesis of R1P from Sucrose and Phosphate

The generation of R1P was carried out at 37° C. in a 100 mM HEPES buffer (pH 7.0) containing 10 mM MgCl₂, 10 mM phosphate, 5 mM NAD(P)⁺, and 50 mM sucrose. The enzymes added (1 U/mL, each) were SP (sucrose phosphorylase, EC 2.4.1.7, SEQ ID Nos: 19-21), PGM (phosphoglucomutase, EC 5.4.2.2, SEQ ID Nos: 118-119), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. RIP concentration was measured by HPLC.

### Example 21: Synthesis of R1P from Cellobiose and Phosphate

The synthesis of R1P was carried out at 37° C. in a 100 mM HEPES buffer (pH 7.0) containing 10 mM MgCl₂, 10 mM phosphate, 5 mM NAD(P)+, and 20 mM cellobiose. The enzymes added (1 U/mL each) were CBP (cellobiose phosphorylase, EC 2.4.1.20, SEQ ID Nos: 22 or 24), PGM (phosphoglucomutase, EC 5.4.2.2, SEQ ID Nos: 118-119), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 22: Synthesis of R1P from Cellodextrin and Phosphate

The synthesis of R1P was carried out at 37° C. in a 100 mM HEPES buffer (pH 7.0) containing 10 mM MgCl₂, 10 mM phosphate, 5 mM NAD⁺, and 30 mM cellodextrins (including cellobiose and long-chain cellodextrins). The enzymes added (1 U/mL each) were CDP (cellodextrin phosphorylase, EC 2.4.1.49, SEQ ID Nos: 23-24), CBP (cellobiose phosphorylase, EC 2.4.1.20, SEQ ID Nos: 22 or 24), PGM (phosphoglucomutase, EC 5.4.2.2, SEQ ID Nos: 118-119), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 23: Synthesis of R1P from D-Glucose and Polyphosphate

The synthesis of R1P was carried out at 37° C. in a 100 mM HEPES buffer (pH 7.0) containing 20 mM MgCl₂, 20 mM polyphosphate, 5 mM NAD(P)+, and 50 mM glucose. The enzymes added (1 U/mL each) were PPGK (polyphosphate glucokinase, EC 2.7.1.63, SEQ ID No: 70), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 24: Synthesis of R1P from D-Fructose and Polyphosphate

The synthesis of R1P was carried out at 37° C. in a 100 mM HEPES buffer (pH 7.0) containing 20 mM MgCl₂, 20 mM polyphosphate, 5 mM NAD(P)*, and 50 mM fructose. The enzymes added (1 U/mL each) were D-XI (xylose isomerase, EC 5.3.1.5, SEQ ID Nos: 111-113), PPGK (polyphosphate glucokinase, EC 2.7.1.63, SEQ ID No: 70), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 25: Synthesis of R1P from Sucrose, Phosphate and Polyphosphate

The synthesis of R1P was carried out at 37° C. in a 100 mM HEPES buffer (pH 7.0) containing 30 mM MgCl₂, 10 mM phosphate, 20 mM polyphosphate, 5 mM NAD(P)+, 50 mM sucrose and 20 mM polyphosphate. The enzymes added (1 U/mL each) were SP (sucrose phosphorylase, EC 2.4.1.7, SEQ ID Nos: 19-21), PGM (phosphoglucomutase, EC 5.4.2.2, SEQ ID Nos: 118-119), D-XI (xylose isomerase, EC 5.3.1.5, SEQ ID Nos: 111-113), PPGK (polyphosphate glucokinase, EC 2.7.1.63, SEQ ID No: 70), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 26: Synthesis of R1P from Starch, Phosphate and Polyphosphate

The synthesis of R1P was carried out at 37° C. in a 100 mM HEPES buffer (pH 7.0) containing 30 mM MgCl₂, 10 mM phosphate, 20 mM polyphosphate, 5 mM NAD(P)⁺, and 50 mM starch (maltodextrin). Starch or maltodextrin may be treated by isoamylase or pullulanase. The enzymes added (1 U/mL, each) were αGP (alpha-glucan phosphorylase, EC 2.4.1.1, SEQ ID Nos: 16-18), PPGK (polyphosphate glucokinase, EC 2.7.1.63, SEQ ID No: 70), PGM (phosphoglucomutase, EC 5.4.2.2, SEQ ID Nos: 118-119), G6PDH (glucose-6-phosphate dehydrogenase, EC 1.1.1.49, SEQ ID Nos: 3-4), 6-phosphogluconolactonase (6PGL, EC 3.1.1.31, SEQ ID No: 94), 6PGDH (6-phosphogluconate dehydrogenase, EC 1.1.1.44, SEQ ID Nos: 1-2), RPI (ribose 5-phosphate isomerase, EC 5.3.1.6, SEQ ID Nos: 114-115), and PPM (phosphopentomutase, EC 5.4.2.7, SEQ ID Nos: 120-122) as well as NAD(P)H oxidase (NOX, EC 1.6.3.4, SEQ ID Nos: 5-7). An aliquot of mixed enzymes was added to the solution to initiate the reaction. The biotransformation was incubated at 37° C. until significant amount of R1P was generated. R1P concentration was measured by HPLC.

### Example 27: ATP Regeneration from Acetyl Phosphate

The regeneration of ATP was carried out at 37° C. in a 100 mM HEPES buffer (pH 7.0) containing 5 mM MgCl₂, 2 mM ADP, and 10 mM acetyl phosphate. The enzyme added (1 U/mL, each) was acetate kinase (AK, EC 2.7.2.1, SEQ ID Nos: 71-73). An aliquot of the enzyme was added to the solution to initiate the reaction. The biotransformation was incubated at 37° C. ATP net regeneration amount was measured by ATP enzymatic kit. Also, glucose and glucose kinase were added to consume regenerated ATP, wherein the product glucose 6-phosphate was measured by coupled with glucose 6-phosphate dehydrogenase in the presence of NAD⁺. The absorbency of NADH at 340 nm was used to calculate the amount of ATP net regeneration.

### Example 28: More ATP Regeneration from ATP

The regeneration of ATP was carried out at 37° C. in a 100 mM HEPES buffer (pH 7.0) containing 5 mM MgCl₂ and 0.1 mM ATP. The enzymes added (1 U/mL, each) were adenylate kinase (ADK, EC 2.7.4.3, SEQ ID Nos: 85-87) and adenosine kinase (AdK, EC 2.7.1.20, SEQ ID Nos: 58-60). ATP net generation amount was measured by ATP enzymatic kit. More ATP regeneration catalyzed by ADK and AdK than that by ADK only. Also, glucose and glucose kinase were added to consume regenerated ATP, wherein the product glucose 6-phosphate was measured by coupled by glucose 6-phosphate dehydrogenase in the presence of NAD⁺. The absorbency of NADH at 340 nm was used to calculate the amount of ATP regeneration.

### Example 29: ATP Regeneration from Polyphosphate

The regeneration of ATP was carried out at 37° C. in a 100 mM HEPES buffer (pH 7.0) containing 10 mM MgCl₂, 1 mM ADP, and 10 mM polyphosphate. The enzyme added (1 U/mL, each) was polyphosphate kinase (PPK, EC 2.7.4.1, SEQ ID Nos: 74-82). An aliquot of the enzyme was added to the solution to initiate the reaction. The biotransformation was incubated at 37° C. ATP generation amount was measured by the ATP enzymatic kit. Also, glucose and glucose kinase were added to consume regenerated ATP, wherein the product glucose 6-phosphate was measured by coupled by glucose 6-phosphate dehydrogenase in the presence of NAD⁺. The absorbency of NADH at 340 nm was used to calculate the amount of ATP regeneration.

### Example 30: ATP Regeneration from Polyphosphate

The regeneration of ATP was carried out at 37° C. in a 100 mM HEPES buffer (pH 7.0) containing 10 mM MgCl₂, 1 mM ADP, and 10 mM polyphosphate. The enzymes added (1 U/mL, each) were polyphosphate: AMP phosphotransferase (PPT, EC 2.7.4.B2; SEQ ID Nos: 83-84) and adenylate kinase (ADK, EC 2.7.4.3; SEQ ID Nos: 85-86). An aliquot of the enzyme was added to the solution to initiate the reaction. The biotransformation was incubated at 37° C. ATP generation amount was measured by ATP enzymatic kit. Also, glucose and glucose kinase were added to consume regenerated ATP, wherein the product glucose 6-phosphate was measured by coupled by glucose 6-phosphate dehydrogenase in the presence of NAD⁺. The absorbency of NADH at 340 nm was used to calculate the amount of ATP regeneration.

### Example 31: ATP Regeneration from Permeabilized Yeast

The regeneration of ATP was carried out at 37° C. in a 100 mM HEPES buffer (pH 7.0) containing 10 mM MgCl₂, 1 mM ADP, and living baker yeast that was treated by freezing-melting. The living yeast cells were gifted by a Chinese beer factory. The yeast cell added was 50 g/L weight cell weight. ATP generation amount of ATP was measured by ATP enzymatic kit. Also, glucose and glucose kinase were added to consume regenerated ATP, wherein the product glucose 6-phosphate was measured by coupled with glucose 6-phosphate dehydrogenase in the presence of NAD⁺. The absorbency of NADH at 340 nm was used to calculate the amount of ATP regeneration.

### Example 32: NMN Synthesis from Inosine Monophosphate and NAM

The synthesis of NMN was carried out at 37° C. in a 100 mM HEPES buffer (pH 7.0) containing 10 mM MgCl₂, 20 mM inosine monophosphate, 2 mM diphosphate, and 20 mM NAM. The enzymes added (1 U/mL, each) were hypoxanthine/guanine phosphoribosyltransferase (HGPRT, EC 2.4.2.8, SEQ ID Nos: 36-45) and nicotinamide phosphoribosyltransferase (NAMPT, EC 2.4.2.12, SEQ ID Nos: 46-52). An aliquot of the mixed enzymes was added to the solution to initiate the reaction. The biotransformation of NMN was incubated at 37° C. NMN concentrations were measured by both HPLC and enzymatic assay.

### Example 33: NMN Synthesis from Guanosine Monophosphate and NAM

The synthesis of NMN was carried out at 37° C. in a 100 mM HEPES buffer (pH 7.0) containing 10 mM MgCl₂, 20 mM guanosine monophosphate, 2 mM diphosphate, and 20 mM NAM. The enzymes added (1 U/mL, each) were hypoxanthine/guanine phosphoribosyltransferase (HGPRT, EC 2.4.2.8, SEQ ID Nos: 36-45) and nicotinamide phosphoribosyltransferase (NAMPT, EC 2.4.2.12, SEQ ID Nos: 46-52). An aliquot of the mixed enzymes was added to the solution to initiate the reaction. The biotransformation of NMN was incubated at 37° C. NMN concentrations were measured by both HPLC and enzymatic assay.

### Example 34: NAD Synthesis from NMN and ATP

The synthesis of NAD was carried out at 37° C. in a 100 mM HEPES buffer (pH 7.0) containing 5 mM MgCl₂, 20 mM NMN and 20 mM ATP. The enzyme added (1 U/mL, each) was nicotinamide nucleotide adenylyltransferase (NMNAT, EC 2.7.7.1, SEQ ID Nos. 88-90). An aliquot of the mixed enzymes was added to the solution to initiate the reaction. The biotransformation of NAD was incubated at 37° C. NAD concentrations were measured by both HPLC and enzymatic assay.

### Example 35: NAD Synthesis from NMN and ATP

The synthesis of NAD was carried out at 37° C. in a 100 mM HEPES buffer (pH 7.0) containing 5 mM MgCl₂, 20 mM NMN, and 20 mM ATP. The enzyme added (1 U/mL, each) was nicotinamide nucleotide adenylyltransferase (NMNAT, EC 2.7.7.1, SEQ ID Nos. 88-90) and diphosphate by diphosphatase (DPP, EC 3.6.1.1, SEQ ID Nos. 105-106). An aliquot of the mixed enzymes was added to the solution to initiate the reaction. The biotransformation of NAD was incubated at 37° C. NAD concentrations were measured by both HPLC and enzymatic assay. More NAD was synthesized when DPP was added.

### Example 36: NADP Synthesis from NMN and ATP

The synthesis of NADP was carried out at 37° C. in a 100 mM HEPES buffer (pH 7.0) containing 10 mM MgCl₂, 20 mM NMN, and 40 mM ATP. The enzymes added (1 U/mL, each) were nicotinamide nucleotide adenylyltransferase (NMNAT, EC 2.7.7.1, SEQ ID Nos. 88-90) and ATP-dependent NAD kinase (NADK, EC 2.7.1.23, SEQ ID Nos. 64-69). An aliquot of the mixed enzymes was added to the solution to initiate the reaction. The biotransformation of NADP was incubated at 37° C. NADP concentration was measured by both HPLC and enzymatic assay.

### Example 37: NADP Synthesis from NMN, ATP and Polyphosphate

The synthesis of NADP was carried out at 37° C. in a 100 mM HEPES buffer (pH 7.0) containing 10 mM MgCl₂, 20 mM NMN, 10 mM ATP and 20 mM polyphosphate. The enzymes added (1 U/mL, each) were nicotinamide nucleotide adenylyltransferase (NMNAT, EC 2.7.7.1, SEQ ID Nos. 88-90) and polyphosphate-dependent NAD kinase (NADK, EC 2.7.1.23, SEQ ID Nos. 64-69). An aliquot of the mixed enzymes was added to the solution to initiate the reaction. The biotransformation of NADP was incubated at 37° C. NADP concentration was measured by both HPLC and enzymatic assay.

### Example 38: NR Synthesis from NMN

The synthesis of NR was carried out at 37° C. in a 100 mM HEPES buffer (pH 7.0) containing 5 mM MgCl₂ and 20 mM NMN. The enzyme added (1 U/mL, each) was 5′-nucleotidase. An aliquot of the mixed enzymes was added to the solution to initiate the reaction. The biotransformation of NR was incubated at 37° C. NR concentration was measured by both HPLC and enzymatic assay.

### Example 39: Enzymatic Removal of Adenosine

The in situ selective removal of adenosine was carried out at 37° C. in a 100 mM HEPES buffer (pH 7.0) containing 5 mM MgCl₂ and 5 mM adenosine. The enzyme added (1 U/mL, each) was adenosine nucleosidase (AN, EC 3.2.2.7, SEQ ID No. 97). An aliquot of the mixed enzymes was added to the solution to initiate the reaction. The hydrolysis of adenosine was incubated at 37° C. D-Ribose concentration was measured by both HPLC equipped with Bio-Rad 87-H column with a refractive index detector. AN may be replaced with purine nucleosidase (PN, EC 3.2.2.1).

### Example 40: Enzymatic Removal of Guanine

The in situ selective removal of guanine was carried out at 37° C. in a 100 mM HEPES buffer (pH 7.0) containing 5 mM MgCl₂ and 5 mM guanine. The enzymes added (1 U/mL, each) were guanine deaminase (GDA, EC 3.5.4.3, SEQ ID Nos. 101-104), xanthine oxidase (XO, EC 1.17.3.2, SEQ ID Nos. 11-15), and catalase (CA, EC 1.11.1.6, EC 1.11.1.21, or EC 1.11.1.7, SEQ ID Nos. 8-10). An aliquot of the mixed enzymes was added to the solution to initiate the reaction. The hydrolysis of adenosine was incubated at 37° C. The concentration of ammonia, a product of GDA, was measured by the ammonia enzymatic kit (Sigma, AA0100-1KT).

### Example 41: Enzymatic Removal of Hypoxanthine

The in situ selective removal of guanine was carried out at 37° C. in a 100 mM HEPES buffer (pH 7.0) containing 5 mM MgCl₂, and 10 mM hypoxanthine. The enzymes added (1 U/mL, each) were xanthine oxidase (XO, EC 1.17.3.2, SEQ ID Nos. 11-15) and catalase (CA, EC 1.11.1.6, EC 1.11.1.21, or EC 1.11.1.7, SEQ ID Nos. 8-10). An aliquot of the mixed enzymes was added to the solution to initiate the reaction. The hydrolysis of hypoxanthine was incubated at 37° C. The disappearance of hypoxanthine was measured by HPLC.

### Example 42: Enzymatic Removal of Hypoxanthine

The in situ selective removal of guanine was carried out at 37° C. in a 100 mM HEPES buffer (pH 7.0) containing 5 mM MgCl₂, and 10 mM hypoxanthine. The enzymes added (1 U/mL, each) were xanthine dehydrogenase (XDH, EC 1.17.3.4, SEQ ID Nos. 11-15), and H₂O-forming NADH oxidase (NOX, EC 1.6.3.4, SEQ ID Nos. 5-7). An aliquot of the mixed enzymes was added to the solution to initiate the reaction. The hydrolysis of hypoxanthine was incubated at 37° C. The disappearance of hypoxanthine was measured by HPLC.

### Example 43: NMN Synthesis from Acetyl Phosphate, NAM, and Inosine Monophosphate

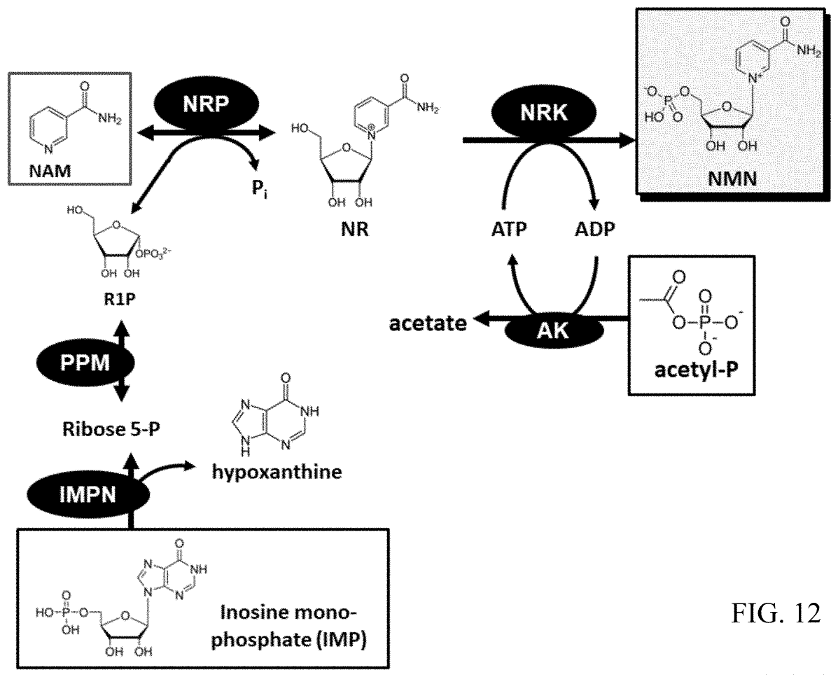

The synthesis of NMN was carried out at 37° C. in a 100 mM HEPES buffer (pH 7.0) containing 10 mM MgCl₂, 0.5 mM MnCl₂, 20 mM NAM, 20 mM inosine monophosphate, 0.5 mM ATP, and 10 mM acetyl phosphate (FIG. 12). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), and acetate kinase (AK, EC 2.7.2.1, SEQ ID Nos 71-73). An aliquot of the mixed enzymes was added to the solution to initiate the reaction. The biotransformation of NMN was incubated at 37° C. NMN concentration was measured by both HPLC and enzymatic assay.

### Example 44: NMN Synthesis from Acetyl Phosphate, NAM and Guanosine Monophosphate

The synthesis of NMN was carried out at 37° C. in a 100 mM HEPES buffer (pH 7.0) containing 10 mM MgCl₂, 0.5 mM MnCl₂, 20 mM NAM, 20 mM guanosine monophosphate, 1 mM ATP, and 10 mM acetyl phosphate. The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), and acetate kinase (AK, EC 2.7.2.1, SEQ ID Nos 71-73). An aliquot of the mixed enzymes was added to the solution to initiate the reaction. The biotransformation of NMN was incubated at 37° C. NMN concentration was measured by both HPLC and enzymatic assay.

### Example 45: NMN Synthesis from ATP, NAM and Inosine Monophosphate

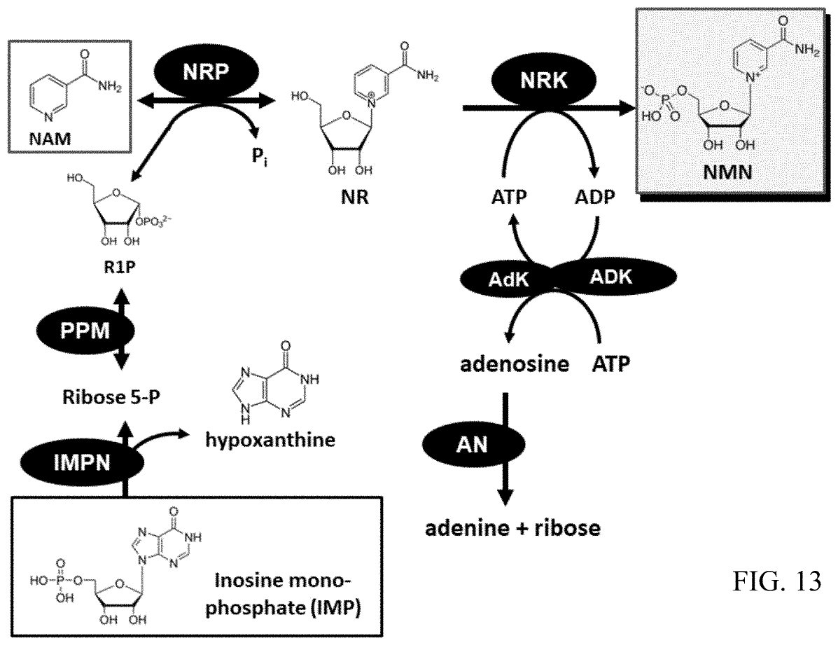

The synthesis of NMN was carried out at 37° C. in a 100 mM HEPES buffer (pH 7.0) containing 10 mM MgCl₂, 0.5 mM MnCl₂, 20 mM NAM, 30 mM inosine monophosphate, and 5 mM ATP (FIG. 13). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), adenylate kinase (ADK, EC 2.7.4.3, SEQ ID Nos 85-87), adenosine kinase (AdK, EC 2.7.1.20, SEQ ID Nos 58-60), and adenosine nucleosidase (AN, EC 3.2.2.7, SEQ ID No 97). An aliquot of the mixed enzymes was added to the solution to initiate the reaction. The biotransformation of NMN was incubated at 37° C. NMN concentration was measured by both HPLC and enzymatic assay and its synthesis was confirmed.

### Example 46: NMN Synthesis from ATP, NAM and Inosine Monophosphate

The synthesis of NMN was carried out at 37° C. in a 100 mM HEPES buffer (pH 7.0) containing 10 mM MgCl₂, 0.5 mM MnCl₂, 20 mM NAM, 30 mM inosine monophosphate, and 5 mM ATP (FIG. 13). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), adenylate kinase (ADK, EC 2.7.4.3, SEQ ID Nos 85-87), adenosine kinase (AdK, EC 2.7.1.20, SEQ ID Nos 58-60), and adenosine nucleosidase (AN, EC 3.2.2.7, SEQ ID No 97). An aliquot of the mixed enzymes was added to the solution to initiate the reaction. The biotransformation of NMN was incubated at 50° C. NMN concentration was measured by both HPLC and enzymatic assay and its synthesis was confirmed. Faster reaction rates were obtained at a reaction temperature of 50° C. than 37° C.

### Example 47: NMN Synthesis from Polyphosphate, NAM and Inosine Monophosphate

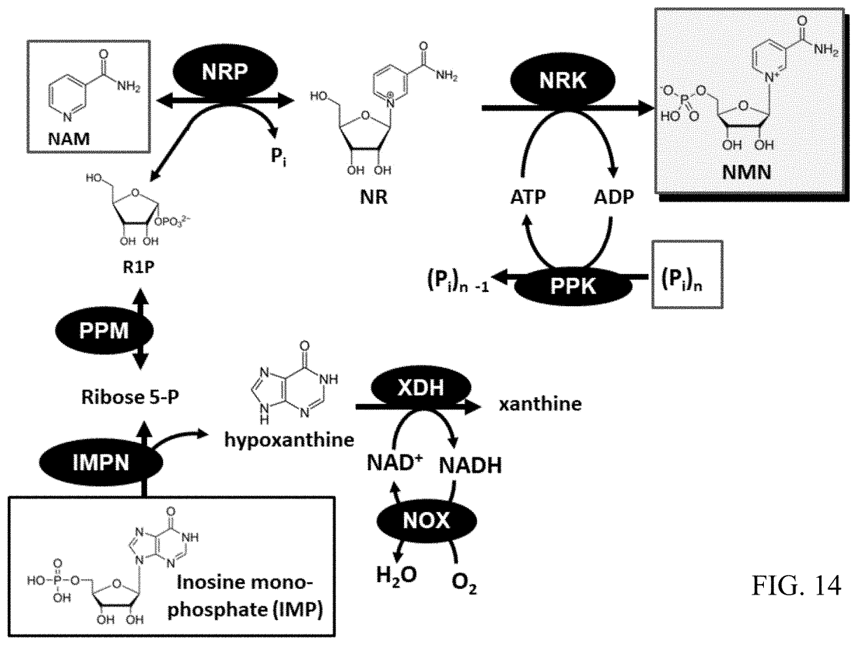

The synthesis of NMN was carried out at 37° C. in a 100 mM HEPES buffer (pH 7.0) containing 20 mM MgCl₂, 0.5 mM MnCl₂, 20 mM NAM, 20 mM inosine monophosphate, 20 mM polyphosphate, and 1 mM ATP (FIG. 14). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), and polyphosphate kinase (PPK, EC 2.7.4.1, SEQ ID Nos 74-82). The biotransformation of NMN was incubated at 37° C. An aliquot of the mixed enzymes was added to the solution to initiate the reaction. NMN concentration was measured by both HPLC and enzymatic assay and its synthesis was confirmed.

### Example 48: NMN Synthesis from Polyphosphate, NAM and Guanosine Monophosphate

The synthesis of NMN was carried out at 37° C. in a 100 mM HEPES buffer (pH 7.0) containing 20 mM MgCl₂, 0.5 mM MnCl₂, 20 mM NAM, 20 mM guanosine monophosphate, 20 mM polyphosphate, and 1 mM ATP. The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), and polyphosphate kinase (PPK, EC 2.7.4.1, SEQ ID Nos 74-82). The biotransformation of NMN was incubated at 37° C. An aliquot of the mixed enzymes was added to the solution to initiate the reaction. NMN concentration was measured by both HPLC and enzymatic assay and its synthesis was confirmed.

### Example 49: NMN Synthesis from Polyphosphate, NAM and Inosine Monophosphate

The synthesis of NMN was carried out at 37° C. in a 100 mM HEPES buffer (pH 7.0) containing 20 mM MgCl₂, 0.5 mM MnCl₂, 20 mM NAM, 20 mM inosine monophosphate, 20 mM polyphosphate, and 1 mM ATP. The enzymes added (1 U/mL, each) were pyrimidine/purine nucleotide 5′-monophosphate nucleosidase (PPMPN, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), polyphosphate kinase (PPK, EC 2.7.4.1, SEQ ID Nos 74-82), xanthine dehydrogenase (XDH, EC 1.17.3.4, SEQ ID Nos. 11-15), and H₂O-forming NADH oxidase (NOX, EC 1.6.3.4, SEQ ID Nos. 5-7). An aliquot of the mixed enzymes was added to the solution to initiate the reaction. NMN concentration was measured by both HPLC and enzymatic assay and its synthesis was confirmed. The supplementary addition of XDH and NOX improved NMN yield.

### Example 50: NMN Synthesis from Polyphosphate, NAM and Inosine Monophosphate

The synthesis of NMN was carried out at 50° C. in a 100 mM HEPES buffer (pH 7.0) containing 20 mM MgCl₂, 0.5 mM MnCl₂, 20 mM NAM, 30 mM inosine monophosphate, 20 mM polyphosphate, and 1 mM ATP (FIG. 14). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1, SEQ ID Nos 25-35), nicotinamide riboside kinase (NRK, EC 2.7.1.22, SEQ ID Nos 61-63), and polyphosphate kinase (PPK, EC 2.7.4.1, SEQ ID Nos 74-82). The biotransformation of NMN was incubated at 50° C. An aliquot of the mixed enzymes was added to the solution to initiate the reaction. NMN concentration was measured by both HPLC and enzymatic assay.

### Example 51: NAD Synthesis from NAM, Inosine Monophosphate, ATP and Polyphosphate

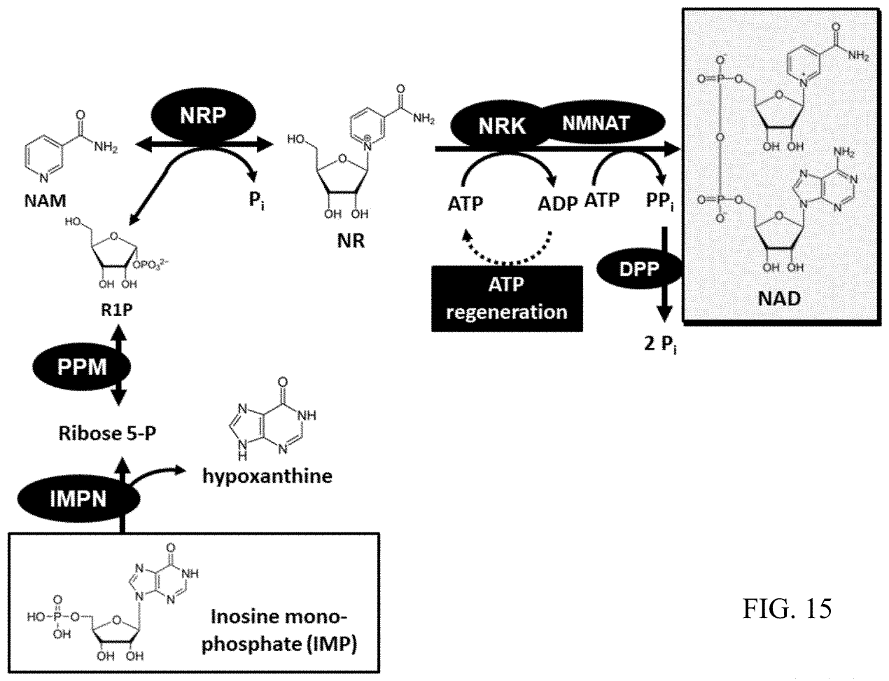

The synthesis of NMN was carried out at 37° C. in a 100 mM HEPES buffer (pH 7.0) containing 30 mM MgCl₂, 0.5 mM MnCl₂, 20 mM NAM, 30 mM inosine monophosphate, 20 mM polyphosphate, and 10 mM ATP (FIG. 15). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1), a bifunctional enzyme (SEQ ID Nos 91-93) including nicotinamide riboside kinase (NRK, EC 2.7.1.22) and nicotinamide nucleotide adenylyltransferase (NMNAT, EC 2.7.7.1), and polyphosphate kinase (PPK, EC 2.7.4.1, SEQ ID Nos 74-82). An aliquot of the mixed enzymes was added to the solution to initiate the reaction. NAD concentration was measured by both HPLC and enzymatic assay and its synthesis was confirmed.

### Example 52: NAD Synthesis from NAM, Inosine Monophosphate, ATP and Polyphosphate

The synthesis of NMN was carried out at 37° C. in a 100 mM HEPES buffer (pH 7.0) containing 20 mM MgCl₂, 0.5 mM MnCl₂, 20 mM NAM, 30 mM inosine monophosphate, 20 mM polyphosphate, and 10 mM ATP (FIG. 15). The enzymes added (1 U/mL, each) were inosinate nucleosidase (IMPN, EC 3.2.2.12, EC 3.2.2.10, SEQ ID Nos 98-100), phosphopentomutase (PPM, EC 5.4.2.7, SEQ ID Nos 120-122), nicotinamide riboside phosphorylase (NRP, EC 2.4.2.1), a bifunctional enzyme (SEQ ID Nos 91-93) including nicotinamide riboside kinase (NRK, EC 2.7.1.22) and nicotinamide nucleotide adenylyltransferase (NMNAT, EC 2.7.7.1), polyphosphate kinase (PPK, EC 2.7.4.1, SEQ ID Nos 74-82), and diphosphatase (DPP, EC 3.6.1.1, SEQ ID Nos. 105-106). An aliquot of the mixed enzymes was added to the solution to initiate the reaction. NAD concentrations were measured by both HPLC and enzymatic assay and its synthesis was confirmed. The additional enzyme DPP improved NAD yield.

### Example 53: NAD Synthesis from NAM, Guanosine Monophosphate, ATP and Polyphosphate

The synthesis of NMN was carried out at 37° C. in a 100 mM HEPES buffer (pH 7.0) containing 20 mM MgCl₂, 0.5 mM MnCl₂, 20 mM NAM, 20 mM guanosine monophosphate, 20 mM polyphosphate, and 10 mM ATP. The enzymes added (1 U/mL, each) were pyrimidine/purine nucleotide 5′-monophosphate nucleosidase (PPMPN, EC 3.2.2.10, SEQ ID Nos: 98-100), phosphopentomutase (PPM, EC 5.4.2.7, SEQ ID Nos 120-122), nicotinamide riboside phosphorylase (NRP, EC 2.4.2.1), a bifunctional enzyme (SEQ ID Nos 91-93) including nicotinamide riboside kinase (NRK, EC 2.7.1.22) and nicotinamide nucleotide adenylyltransferase (NMNAT, EC 2.7.7.1), polyphosphate kinase (PPK, EC 2.7.4.1, SEQ ID Nos 74-82), and diphosphatase (DPP, EC 3.6.1.1, SEQ ID Nos. 105-106). An aliquot of the mixed enzymes was added to the solution to initiate the reaction. NAD concentration was measured by both HPLC and enzymatic assay.

### Example 54: NMN Synthesis from NAM and Inosine Monophosphate

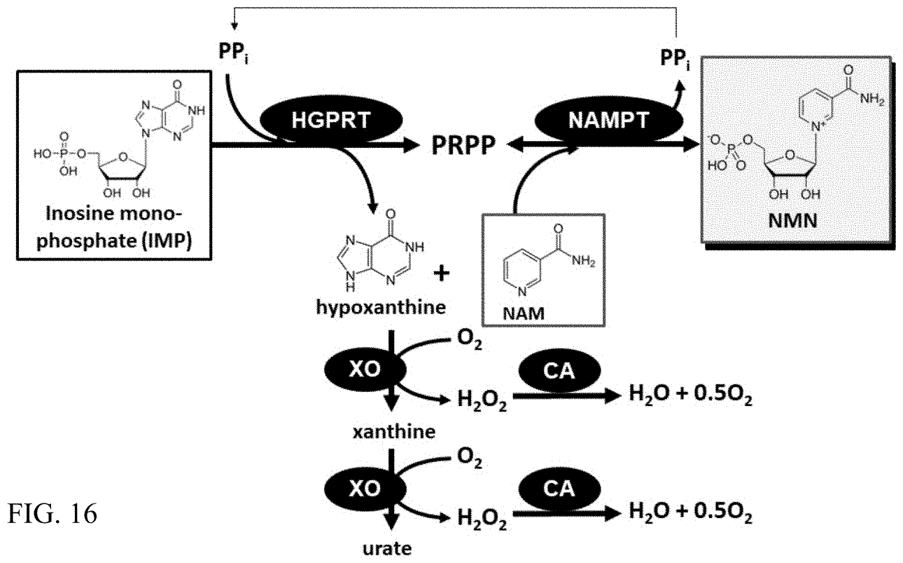

The synthesis of NMN was carried out at 37° C. in a 100 mM HEPES buffer (pH 7.0) containing 10 mM MgCl₂, 0.5 mM MnCl₂, 20 mM NAM, and 20 mM inosine monophosphate (FIG. 16). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), xanthine oxidase (XO, EC 1.17.3.2, SEQ ID Nos. 11-15) and catalase (CA, EC 1.11.1.6, EC 1.11.1.21, or EC 1.11.1.7, SEQ ID Nos. 8-10). An aliquot of the mixed enzymes was added to the solution to initiate the reaction. NMN concentrations were measured by both HPLC and enzymatic assay and its synthesis was confirmed. The supplementary addition of XO/CA enhanced NMN yield in comparison of the synthesis of NMN catalyzed by HGPRT/NAMPT.

### Example 55: NMN Synthesis from NAM and Inosine Monophosphate

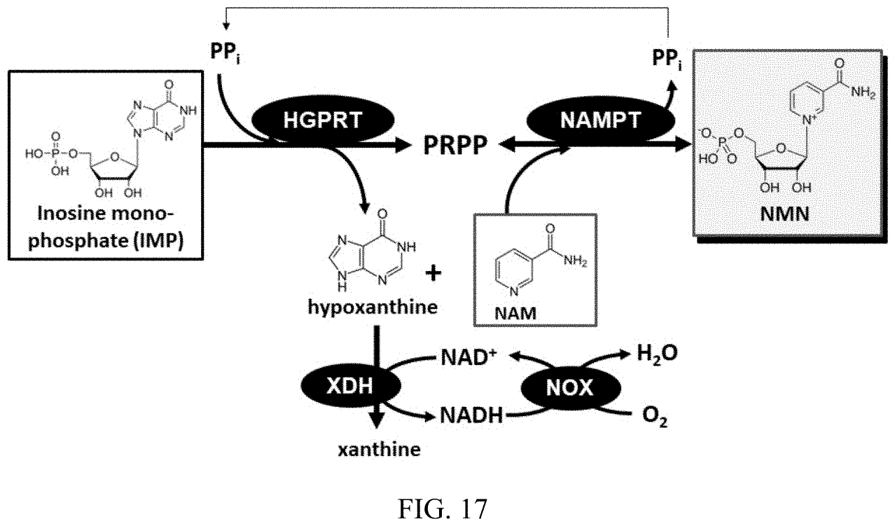

The synthesis of NMN was carried out at 37° C. in a 100 mM HEPES buffer (pH 7.0) containing 10 mM MgCl₂, 0.5 mM MnCl₂, 20 mM NAM, 20 mM inosine monophosphate and 1 mM NAD (FIG. 17). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), xanthine dehydrogenase (XDH, EC 1.17.3.4, SEQ ID Nos. 11-15) and H₂O-forming NADH oxidase (NOX, EC 1.6.3.4, SEQ ID Nos. 5-7). An aliquot of the mixed enzymes was added to the solution to initiate the reaction. NMN concentration was measured by both HPLC and enzymatic assay and its synthesis was confirmed. The supplementary addition of XDH/NOX enhanced NMN yield in comparison of the synthesis of NMN catalyzed by HGPRT/NAMPT.

### Example 56: NMN Synthesis from NAM and Guanosine Monophosphate

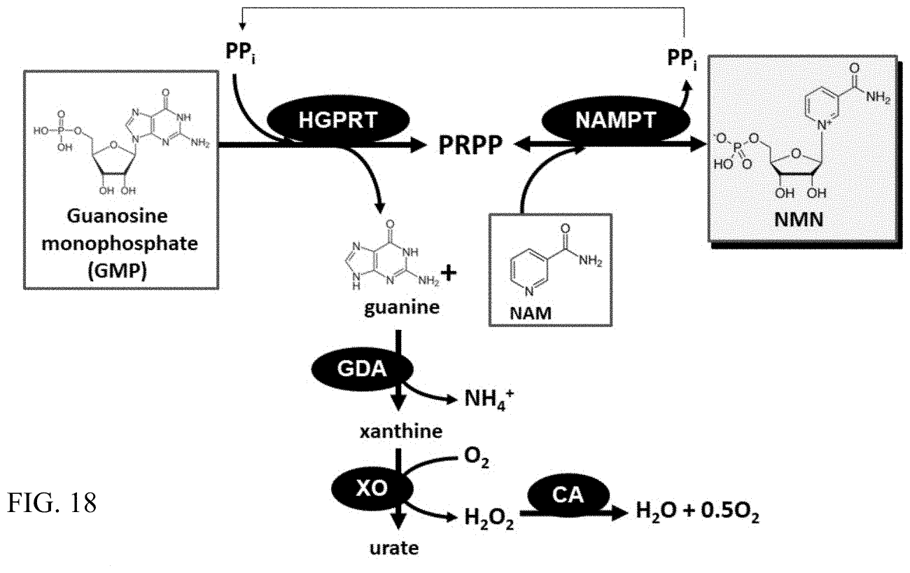

The synthesis of NMN was carried out at 37° C. in a 100 mM HEPES buffer (pH 7.0) containing 10 mM MgCl₂, 0.5 mM MnCl₂, 20 mM NAM, and 20 mM guanosine monophosphate (FIG. 18). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), guanine deaminase (GDA, EC 3.5.4.3, SEQ ID Nos. 101-104), xanthine dehydrogenase (XDH, EC 1.17.3.4, SEQ ID Nos. 11-15), and H₂O-forming NADH oxidase (NOX, EC 1.6.3.4, SEQ ID Nos. 5-7). An aliquot of the mixed enzymes was added to the solution to initiate the reaction. NMN concentration was measured by both HPLC and enzymatic assay and its synthesis was confirmed.

### Example 57: NAD Synthesis from NAM, ATP and Inosine Monophosphate

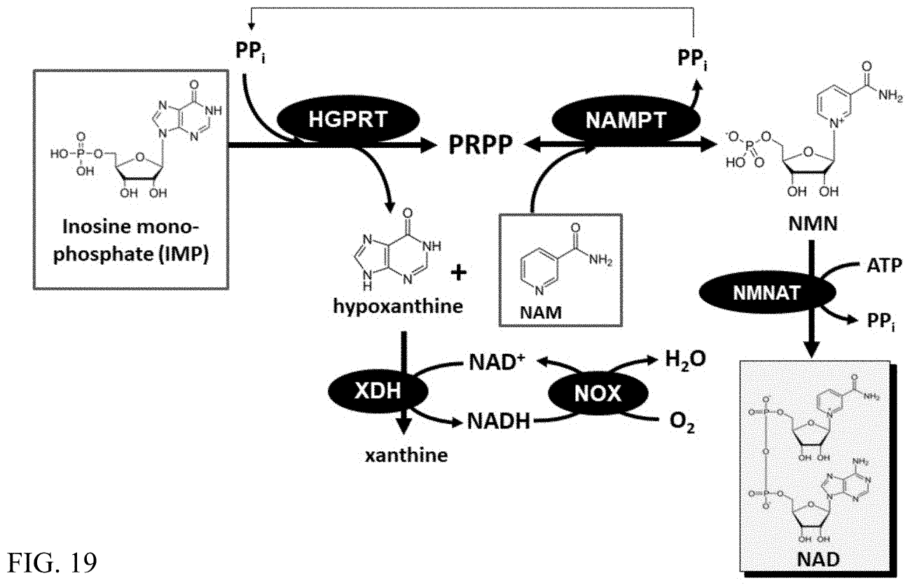

The synthesis of NAD was carried out at 37° C. in a 100 mM HEPES buffer (pH 7.0) containing 10 mM MgCl₂, 0.5 mM MnCl₂, 20 mM NAM, 20 mM inosine monophosphate, 10 mM ATP, and 0.05 mM NAD⁺ (FIG. 19). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), nicotinamide nucleotide adenylyltransferase (NMNAT, EC 2.7.7.1, SEQ ID Nos 88-90), xanthine dehydrogenase (XDH, EC 1.17.3.4, SEQ ID Nos. 11-15), and H₂O-forming NADH oxidase (NOX, EC 1.6.3.4, SEQ ID Nos. 5-7). An aliquot of the mixed enzymes was added to the solution to initiate the reaction. NAD concentration was measured by both HPLC and enzymatic assay and its synthesis was confirmed.

### Example 58: NAD Synthesis from NAM, ATP and Inosine Monophosphate

The synthesis of NAD was carried out at 37° C. in a 100 mM HEPES buffer (pH 7.0) containing 10 mM MgCl₂, 0.5 mM MnCl₂, 20 mM NAM, 20 mM inosine monophosphate and 10 mM ATP (FIG. 19). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), nicotinamide nucleotide adenylyltransferase (NMNAT, EC 2.7.7.1, SEQ ID Nos 88-90), xanthine dehydrogenase (XDH, EC 1.17.3.4, SEQ ID Nos. 11-15), and H₂O-forming NADH oxidase (NOX, EC 1.6.3.4, SEQ ID Nos. 5-7). An aliquot of the mixed enzymes was added to the solution to initiate the reaction. NAD concentration was measured by both HPLC and enzymatic assay and its synthesis was confirmed. Supplementary addition of a small amount of DPP (e.g., ˜0.01 U/mL) increased NAD yield.

### Example 59: NAD Synthesis from NAM, ATP and Guanosine Monophosphate

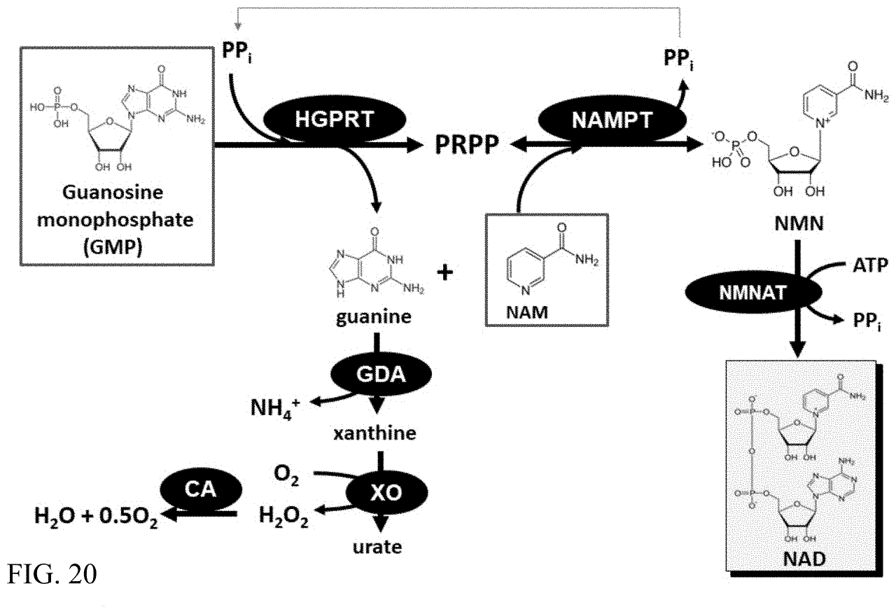

The synthesis of NAD was carried out at 37° C. in a 100 mM HEPES buffer (pH 7.0) containing 10 mM MgCl₂, 0.5 mM MnCl₂, 20 mM NAM, 20 mM guanosine monophosphate, and 10 mM ATP (FIG. 20). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), nicotinamide nucleotide adenylyltransferase (NMNAT, EC 2.7.7.1, SEQ ID Nos 88-90), guanine deaminase (GDA, EC 3.5.4.3, SEQ ID Nos. 101-104), xanthine oxidase (XO, EC 1.17.3.2, SEQ ID Nos. 11-15) and catalase (CA, EC 1.11.1.6, EC 1.11.1.21, or EC 1.11.1.7, SEQ ID Nos. 8-10). An aliquot of the mixed enzymes was added to the solution to initiate the reaction. NAD concentration was measured by both HPLC and enzymatic assay and its synthesis was confirmed. A combination of XO/CA may be replaced with a combination of GDH/NOX.

### Example 60: NADP Synthesis from NAM, ATP, Polyphosphate, and Inosine Monophosphate

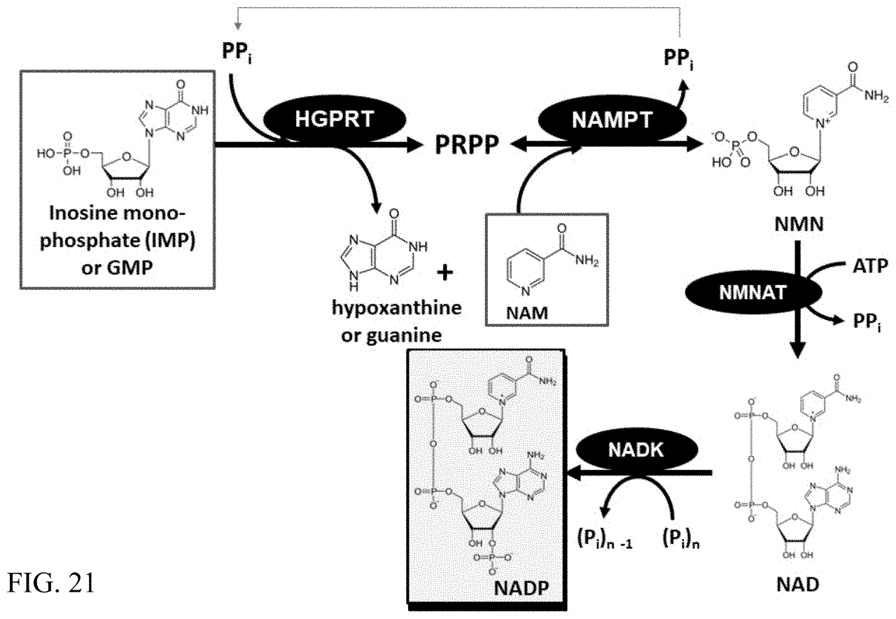

The synthesis of NADP was carried out at 37° C. in a 100 mM HEPES buffer (pH 7.0) containing 30 mM MgCl₂, 0.5 mM MnCl₂, 20 mM NAM, 20 mM polyphosphate, 20 mM inosine monophosphate, and 10 mM ATP (FIG. 21). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), nicotinamide nucleotide adenylyltransferase (NMNAT, EC 2.7.7.1, SEQ ID Nos 88-90), and polyphosphate-dependent NAD kinase (NADK, EC 2.7.1.23, SEQ ID Nos. 64-69). An aliquot of the mixed enzymes was added to the solution to initiate the reaction. NADP concentration was measured by both HPLC and enzymatic assay and its synthesis was confirmed.

### Example 61: NADP Synthesis from NAM, ATP, Polyphosphate, and Inosine Monophosphate

The synthesis of NADP was carried out at 37° C. in a 100 mM HEPES buffer (pH 7.0) containing 30 mM MgCl₂, 0.5 mM MnCl₂, 20 mM NAM, 20 mM polyphosphate, 20 mM inosine monophosphate and 10 mM ATP (FIG. 21). The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), nicotinamide nucleotide adenylyltransferase (NMNAT, EC 2.7.7.1, SEQ ID Nos 88-90), polyphosphate-dependent NAD kinase (NADK, EC 2.7.1.23, SEQ ID Nos. 64-69), xanthine dehydrogenase (XDH, EC 1.17.3.4, SEQ ID Nos. 11-15) and H₂O-forming NADH oxidase (NOX, EC 1.6.3.4, SEQ ID Nos. 5-7). An aliquot of the mixed enzymes was added to the solution to initiate the reaction. NADP concentration was measured by both HPLC and enzymatic assay and its synthesis was confirmed. The supplementary addition of XDH and NOX enhanced NADP yield slightly.

### Example 62: NADP Synthesis from NAM, ATP, Polyphosphate, and Guanosine Monophosphate

The synthesis of NADP was carried out at 37° C. in a 100 mM HEPES buffer (pH 7.0) containing 30 mM MgCl₂, 0.5 mM MnCl₂, 20 mM NAM, 20 mM polyphosphate, 20 mM guanosine monophosphate and 10 mM ATP. The enzymes added (1 U/mL, each) were hypoxanthine/guanine phosphoribosyltransferase (HGPRT, EC 2.4.2.8, SEQ ID Nos 36-45), nicotinamide phosphoribosyltransferase (NAMPT, EC 2.4.2.12, SEQ ID Nos 46-52), nicotinamide nucleotide adenylyltransferase (NMNAT, EC 2.7.7.1, SEQ ID Nos 88-90), and polyphosphate-dependent NAD kinase (NADK, EC 2.7.1.23, SEQ ID Nos. 64-69). An aliquot of the mixed enzymes was added to the solution to initiate the reaction. NADP concentration was measured by both HPLC and enzymatic assay and its synthesis was confirmed.

### XI. Tables

TABLE 1
List of exemplary enzymes and their UniProt IDs and amino acid lengths included in the
current disclosure.

| | | | | | |
|---|---|---|---|---|---|
| | | | | Length | Seq. |
| EC | Enzyme | Organism | UniProt | (AA) | No. |

1.1.1.44. 6-Phosphogluconate dehydrogenase (6PGDH): 6-phosphogluconate + NAD(P)⁺ =
NAD(P)H + ribulose 5-phosphate

| | | | | | |
|---|---|---|---|---|---|
| 1.1.1.4 | 6PGDH | Thermotoga maritima | Q9WYR9 | 469 | 1 |
| 4 | | | | | |
| 1.1.1.4 | 6PGDH | Moorella thermoacetica | Q2RIZ2 | 298 | 2 |
| 4 | | | | | |

1.1.1.49. Glucose 6-phosphate dehydrogenase (G6PDH): G6P + NAD(P)⁺ = NAD(P)H + 6-
phosphogluconolactone

| | | | | | |
|---|---|---|---|---|---|
| 1.1.1.4 | G6PDH | Thermotoga maritima | Q9X0N9 | 496 | 3 |
| 9 | | | | | |
| 1.1.1.4 | G6PDH | Zymomonas mobilis | P21907 | 485 | 4 |
| 9 | | | | | |

1.6.3.4. NADH oxidase (H₂O-forming) (NOX): NADH + ½ O₂ = NAD⁺ + H₂O

| | | | | | |
|---|---|---|---|---|---|
| 1.6.3.4 | NOX | Clostridium aminovalericum | Q2WFW5 | 448 | 5 |
| 1.6.3.4 | NOX | Clostridium acetobutylicum | Q97K92 | 392 | 6 |
| 1.6.3.4 | NOX | Streptococcus mutans | I6L920 | 457 | 7 |

1.11.1.6 Catalase (CA), 1.11.1.7, 1.11.1.21 (catalase-peroxidase): H₂O₂ = H₂O + 0.5 O₂

| | | | | | |
|---|---|---|---|---|---|
| 1.11.1. | CA | Thermus brockianus | Q596K8 | 286 | 8 |
| 6 | | | | | |
| 1.11.1. | CA | Bacillus pumilus | A0A143G97 | 491 | 9 |
| 6 | | | 6 | | |
| 1.11.1. | CA | Geobacillus stearothermophilus | A0A0K2HCN | 736 | 10 |
| 7 | | | 2 | | |

1.17.1.4. Xanthine dehydrogenase (XDH): xanthine + NAD⁺ = urate + NADH
1.7.3.2. Xanthine oxidase (XO): hypoxanthine + H₂O + O₂ = xanthine + H₂O₂; xanthine + H₂O +
O₂ = urate + H₂O₂

| | | | | | |
|---|---|---|---|---|---|
| 1.17.1. | XDH | Bos taurus (bovine) | P80457 | 1332 | 11 |
| 4 | | | | | |
| 1.17.1. | XDH | Homo sapiens | P47989 | 1333 | 12 |
| 4 | | | | | |
| 1.17.1. | XDH | Rattus norvegicus | P22985 | 1331 | 13 |
| 4 | | | | | |
| 1.17.1. | XDH | E. coli K-12 | Q46799; + | 752 | 14 |
| 4 | | | Q46800; + | 292 | |
| | | | Q46801 | 159 | |
| 1.7.3.2 | XDH | Blastobotrys adeninivorans | R4ZGN4 | 1405 | 15 |

2.4.1.1. alpha-glucan phosphorylase (αGP): (glu)ₙ + Pᵢ = (glu)ₙ₋₁ + glucose 1-phosphate

| | | | | | |
|---|---|---|---|---|---|
| 2.4.1.1 | αGp | Clostridium thermocellum | A3DCB6 | 855 | 16 |
| 2.4.1.1 | αGp | Thermotoga neapolitana | B9K9F0 | 823 | 17 |
| 2.4.1.1 | αGp | Thermococcus kodakarensis KOD1 | Q5JH18 | 830 | 18 |

2.4.1.7. sucrose phosphorylase (SP): sucrose + Pᵢ = glucose 1-phosphate + fructose

| | | | | | |
|---|---|---|---|---|---|
| 2.4.1.7 | SP | Bifidobacterium adolescentis | Q84H02 | 504 | 19 |
| 2.4.1.7 | SP | Leuconostoc mesenteroides | Q59495 | 490 | 20 |
| 2.4.1.7 | SP | Thermoanaerobacterium | D9TT09 | 488 | 21 |
| | | thermosaccharolyticum | | | |

2.4.1.20. Cellobiose phosphorylase (CBP): (glu)ₙ + Pᵢ = (glu)ₙ₋₁ + glucose 1-phosphate
2.4.1.49. Cellodextrin phosphorylase (CDP)

| | | | | | |
|---|---|---|---|---|---|
| 2.4.1.2 | CBP | Clostridium thermocellum | A3DC35 | 811 | 22 |
| 0 | | | | | |
| 2.4.1.4 | CDP | Clostridium thermocellum | O24780 | 980 | 23 |
| 9 | | | | | |
| 2.4.1.2 | CBP + | Thermosipho africanus | B7IED6 | 1019 | 24 |
| 0 | CDP | | | | |
| 2.4.1.4 | | | | | |
| 9 | | | | | |

2.4.2.1. Nicotinamide riboside phosphorylase (NRP): NAM + R1P = NR + Pᵢ

| | | | | | |
|---|---|---|---|---|---|
| 2.4.2.1 | NRP | Cellulomonas sp. | P81989 | 282 | 25 |
| 2.4.2.1 | NRP | Bos taurus (Beef liver) | P55859 | 289 | 26 |
| 2.4.2.1 | NRP | Homo sapiens | P00491 | 289 | 27 |
| 2.4.2.1 | NRP | Escherichia coli (strain K12) | P45563 | 277 | 28 |
| | (XapA) | | | | |
| 2.4.2.1 | PNP | Bacillus halodurans | Q9KCN8 | 275 | 29 |
| 2.4.2.1 | PNP | Thermus thermophilus | Q72L69 | 275 | 30 |
| 2.4.2.1 | PNP | Thermotoga maritima MSB8 | Q9X1T2 | 265 | 31 |
| 2.4.2.1 | PNP | Meiothermus silvanus | D7BIF5 | 275 | 32 |
| 2.4.2.1 | PNP | Clostridium thermocellum | A3DEQ4 | 275 | 33 |
| 2.4.2.1 | PNP | Geobacillus thermodenitrificans | A4IN93 | 236 | 34 |
| 2.4.2.1 | PNP | Deinococcus geothermalis | Q1IY92 | 261 | 35 |

2.4.2.8. Hypoxanthine/guanine phosphoribosyltransferase (HGPRT): IMP + PPᵢ = hypoxanthine +
PRPP

| | | | | | |
|---|---|---|---|---|---|
| 2.4.2.8 | HGPRT | Giardia intestinalis ATCC 50803 | E2RTU5 | 230 | 36 |
| 2.4.2.8 | HGPRT | Trypanosoma cruzi | Q4DGA3; | 221 | 37 |
| | | | Q4DRC4 | 241 | 38 |
| 2.4.2.8 | HGPRT | Gallus gallus (chicken) | Q9W719 | 218 | 39 |
| 2.4.2.8 | HGPRT | Clostridium thermocellum | A3DHM7 | 184 | 40 |
| 2.4.2.8 | HGPRT | Haloferax volcanii | D4GZW3 | 237 | 41 |
| | | | D4GVR1 | 188 | 42 |
| | | | D4GVW6 | 181 | 43 |
| 2.4.2.8 | HGPRT | Halobacterium salinarum | Q9HRT1 | 188 | 44 |
| | | | Q9HNG3 | 239 | 45 |

2.4.2.12. Nicotinamide phosphoribosyltransferase (NAMPT): NAM + PRPP = NMN + PPᵢ

| | | | | | |
|---|---|---|---|---|---|
| 2.4.2.1 | NAMPT | Haemophilus ducreyi | G1U9V7 | 495 | 46 |
| 2 | | | | | |
| 2.4.2.1 | NAMPT | Shewanella oneidensis | Q8EFJ1 | 490 | 47 |
| 2 | | | | | |
| 2.4.2.1 | NAMPT | Homo sapiens | P43490 | 491 | 48 |
| 2 | | | | | |
| 2.4.2.1 | NAMPT | Meiothermus ruber DSM 1279 | D3PPH8 | 463 | 49 |
| 2 | | | | | |
| 2.4.2.1 | NAMPT | Tenacibaculum maritimum | A0A2H1E8C | 485 | 50 |
| 2 | | | 7 | | |
| 2.4.2.1 | NAMPT | Mariyirga tractuosa | E4TQE1 | 488 | 51 |
| 2 | | | | | |
| 2.4.2.1 | NAMPT | Synechocystis sp. PCC 6803 | Q55929 | 462 | 52 |
| 2 | | | | | |

2.7.1.15. Ribokinase (RK): ribose + ATP = ribose 5-phosphate (RSP) + ADP

| | | | | | |
|---|---|---|---|---|---|
| 2.7.1.1 | RK | Escherichia coli (strain K12) | P0A9J6 | 309 | 53 |
| 5 | | | | | |
| 2.7.1.1 | RK | Thermoanaerobacterium | D9TQJ0 | 315 | 54 |
| 5 | | thermosaccharolyticum | | | |
| 2.7.1.1 | RK | Thermus thermophilus | Q72116 | 306 | 55 |
| 5 | | | | | |

2.7.1.17. D-xylulokinase (XK): D-xylulose + ATP = D-xylulose 5-phopshate + ADP

| | | | | | |
|---|---|---|---|---|---|
| 2.7.1.1 | XK | Thermotoga maritima | Q9WXX1 | 492 | 56 |
| 7 | | | | | |
| 2.7.1.1 | XK | Geobacillus stearothermophilus | A0A0K2HD7 | 523 | 57 |
| 7 | | | 4 | | |

2.7.1.20. Adenosine kinase (AdK): AMP + ADP = ATP + adenosine

| | | | | | |
|---|---|---|---|---|---|
| 2.7.1.2 | AdK | Myceliophthora thermophila | G2QBY2 | 348 | 58 |
| 0 | | | | | |
| 2.7.1.2 | AdK | Thermothielavioides terrestris | G2RAX0 | 347 | 59 |
| 0 | | | | | |
| 2.7.1.2 | AdK | Saccharomyces cerevisiae | P47143 | 340 | 60 |
| 0 | | | | | |

2.7.1.22. Nicotinamide riboside kinase (NRK): NR+ ATP = NMN + ADP

| | | | | | |
|---|---|---|---|---|---|
| 2.7.1.2 | NRK | Saccharomyces cereyisioe | P53915 | 240 | 61 |
| 2 | | | | | |
| 2.7.1.2 | NRK | Myceliophthora thermophila | G2QPS1 | 348 | 62 |
| 2 | | | | | |
| 2.7.1.2 | NRK | Pseudomonas alcaligenes | A0A1N6WJ3 | 173 | 63 |
| 2 | | | 2 | | |

2.7.1.23. NAD kinase (NADK): NAD + ATP = NADP + ADP;
NAD + poly(P)ₙ = NADP + poly(P)ₙ₋₁

| | | | | | |
|---|---|---|---|---|---|
| 2.7.1.2 | NADK | Clostridium thermocellum | A3DDM2 | 289 | 64 |
| 3 | | | | | |
| 2.7.1.2 | NADK | Thermotoga maritima | Q9X255 | 258 | 65 |
| 3 | | | | | |
| 2.7.1.2 | NADK | Thermococcus kodakarensis | Q5JEW5 | 278 | 66 |
| 3 | | | | | |
| 2.7.1.2 | NADK | Pyrococcus horikoshii | O58801 | 277 | 67 |
| 3 | | | | | |
| 2.7.1.2 | NADK | Thermoanaerobacterium saccharolyticum | I3VWN0 | 272 | 68 |
| 3 | | | | | |
| 2.7.1.2 | NADK | Microroccus luteus | Q8VUL9 | 362 | 69 |
| 3 | | | | | |

2.7.1.63. Polyphosphate glucokinase (PPGK): poly(P)ₙ + glucose = G6P + poly(P)ₙ₋₁

| | | | | | |
|---|---|---|---|---|---|
| 2.7.1.6 | PPGK | Thermobifida fusca | Q47NX5 | 262 | 70 |
| 3 | | | | | |

2.7.2.1. Acetate kinase (AK): acetyl-phosphate + ADP = acetate + ATP

| | | | | | |
|---|---|---|---|---|---|
| 2.7.2.1 | AK | Geobacillus stearothermophilus | A0A0K2H6Q | 396 | 71 |
| | | | 6 | | |
| 2.7.2.1 | AK | Methanosarcina thermophila | A0A0E3NHU | 408 | 72 |
| | | | 1 | | |
| 2.7.2.1 | AK | Thermotoga maritima | Q9WYB1 | 403 | 73 |

2.7.4.1 Polyphosphate kinase (PPK): ADP + poly(P)ₙ = ATP + poly(P)ₙ₋₁

| | | | | | |
|---|---|---|---|---|---|
| 2.7.4.1 | PPK | Corynebacterium glutamicum ATCC | Q8NM65 | 306 | 74 |
| | | 13032 | Q8NRX4 | 320 | 75 |
| 2.7.4.1 | PPK | Cytophaga hutchinsonii | A0A6N4SM | 305 | 76 |
| | | | B5 | | |
| 2.7.4.1 | PPK | Deinococcus radiodurans | Q9RY20 | 266 | 77 |
| 2.7.4.1 | PPK | Meiothermus ruber | M9XB82 | 267 | 78 |
| 2.7.4.1 | PPK | Deinococcus geothermalis | Q1IW43 | 267 | 79 |
| 2.7.4.1 | PPK | Mycobacterium tuberculosis | O05877 | 295 | 80 |
| 2.7.4.1 | PPK | Pseudomonas aeruginosa ATCC 15692 | Q9I6Z1 | 357 | 81 |
| 2.7.4.1 | PPK | Thermus thermophilus HB27 | Q72JY1 | 608 | 82 |

2.7.4.B2. Polyphosphate:AMP phosphotransferase (PPT): AMP + poly(P)ₙ = ADP + poly(P)ₙ₋₁

| | | | | | |
|---|---|---|---|---|---|
| 2.7.4.B | PPT | Acinetobacter johnsonii 210A | Q83XD3 | 475 | 83 |
| 2 | | | | | |
| 2.7.4.B | PPT | Myxococcus xanthus | Q4JGY7 | 259 | 84 |
| 2 | | | | | |

2.7.4.3. Adenylate kinase (ADK): 2 ADP = ATP + AMP

| | | | | | |
|---|---|---|---|---|---|
| 2.7.4.3 | ADK | Clostridium thermocellum | A3DJJ3 | 217 | 85 |
| 2.7.4.3 | ADK | Sulfolobus acidocaldarius | Q4JBH4 | 189 | 86 |
| 2.7.4.3 | ADK | Thermotoga maritima | Q9X1I8 | 220 | 87 |

2.7.7.1. Nicotinamide nucleotide adenylyltransferase (NMNAT): NMN + ATP = NAD + PPᵢ

| | | | | | |
|---|---|---|---|---|---|
| 2.7.7.1 | NMNAT | Methanocaldococcus jannaschii | Q57961 | 168 | 88 |
| 2.7.7.1 | NMNAT | Saccharomyces cerevisiae | Q06178 | 401 | 89 |
| 2.7.7.1 | NMNAT | Leishmania braziliensis | A4H990 | 307 | 90 |
| 2.7.7.1 + | NMNAT + | Clostridium thermocellum | A3DJ48 | 340 | 91 |
| 2.7.1. | NRK | | | | |
| 22 | | | | | |
| 2.7.7.1 + | NMNAT + | Escherichia coli (strain K12) | P27278 | 410 | 92 |
| 2.7.1. | NRK | | | | |
| 22 | | | | | |
| 2.7.7.1 + | NMNAT + | Salmonella typhimurium | P24518 | 410 | 93 |
| 2.7.1. | NRK | | | | |
| 22 | | | | | |

3.1.1.31. 6-phosphogluconolactonage (6PGL): 6-phosphogluconolactone + H₂O = 6-
phosphogluconante

| | | | | | |
|---|---|---|---|---|---|
| 3.1.1.3 | 6PGL | Thermotoga maritima | Q9X0N8 | 220 | 94 |
| 1 | | | | | |

3.2.2.7 Adenosine nucleosidase (AN), 3.2.2.1 (purine nucleosidase, PN): adenosine + H₂O =
adenine + ribose

| | | | | | |
|---|---|---|---|---|---|
| 3.2.2.1 | PN | Trypanosoma brucei brucei | Q57ZL6 | 327 | 95 |
| 3.2.2.1 | PN | Bacillus thuringiensis | A7UHH1 | 321 | 96 |
| 3.2.2.7 | AN | Coffea arabica | NA | | |
| 3.2.—.— | AN | Escherichia coli K-12 MG1655 | P22564 | 304 | 97 |

3.2.2.10. Pyrimidine/purine nucleotide monophosphate nucleosidase (PPMPN): purine 5′-
nucleotide + H₂O = purine base + R5P

| | | | | | |
|---|---|---|---|---|---|
| 3.2.2.1 | PPMPN | Escherichia coli K-12 MG1655 | P0ADR8 | 454 | 98 |
| 0 | | | | | |
| 3.2.2.1 | PPMPN | Shigella flexneri | P0ADS1 | 454 | 99 |
| 0 | | | | | |
| 3.2.2.1 | PPMPN | Mannheimia succiniciproducens | Q65TN5 | 485 | 100 |
| 0 | | | | | |

3.5.4.3. Guanine deaminase (GDA): guanine + H₂O + H⁺ = NH₄⁺ + xanthine

| | | | | | |
|---|---|---|---|---|---|
| 3.5.4.3 | GDA | Bacillus subtilis 168 | O34598 | 156 | 101 |
| 3.5.4.3 | GDA | Haloferax volcanii | D4GTA3 | 337 | 102 |
| | | | D4GXV5 | 437 | 103 |
| 3.5.4.3 | GDA | Halobacterium salinarum | Q9HR24 | 134 | 104 |

3.6.1.1. Inorganic diphosphatase (DPP): pyrophosphate + H₂O = 2 phosphate

| | | | | | |
|---|---|---|---|---|---|
| 3.6.1.1 | DPP | Thermoplasma acidophilum | P37981 | 179 | 105 |
| 3.6.1.1 | DPP | Pyrococcus furiosus | Q8U438 | 178 | 106 |

5.1.3.1. D-xylulose 5-phosphate 3-epimerase (RuPE): D-xylulose 5-phosphate = D-ribulose 5-
phosphate

| | | | | | |
|---|---|---|---|---|---|
| 5.1.3.1 | RuPE | Clostridium thermocellum | A3DCY3 | 220 | 107 |
| 5.1.3.1 | RuPE | Thermotoga maritima | Q9X243 | 220 | 108 |

5.1.3.31. Pentose 3-epimerase (P3E): D-xylulose = D-ribulose

| | | | | | |
|---|---|---|---|---|---|
| 5.1.3.3 | P3E | Pseudomonas cichorii | A0A3M4WA | 224 | 109 |
| 1 | | | L7 | | |
| 5.1.3.3 | P3E | Rhodobacter sphaeroides | Q31W04 | 295 | 110 |
| 1 | | | | | |

5.3.1.5. D-xylose isomerase (D-XI): D-xylose = D-xylulose

| | | | | | |
|---|---|---|---|---|---|
| 5.3.1.5 | D-XI | Thermus thermophilus | P26997 | 387 | 111 |
| 5.3.1.5 | D-XI | Thermotoga neapolitana | B9KBM8 | 307 | 112 |
| 5.3.1.5 | D-XI | Geobacillus stearothermophilus | P54273 | 441 | 113 |

5.3.1.6. Ribose 5-phosphate isomerase (RPI): D-ribulose 5-phosphate = D-ribose 5-phosphate

| | | | | | |
|---|---|---|---|---|---|
| 5.3.1.6 | RPI | Clostridium thermocellum | A3DIL8 | 149 | 114 |
| 5.3.1.6 | RPI | Thermotoga maritima | Q9X0G9 | 143 | 115 |

5.3.1.8. Mannose 6-phosphate isomerase (MPI): D-ribulose = D-ribose
5.3.1.20. D-ribose isomerase (D-RI): D-ribulose = D-ribose

| | | | | | |
|---|---|---|---|---|---|
| 5.3.1.8 | MPI | Geobacillus thermodenitrificans | A4ITT1 | 320 | 116 |
| 5.3.1.2 | D-RI | Mycobacterium smegmatis | I7G3K2 | 387 | 117 |
| 0 | | | | | |

5.4.2.2. Phosphoglucomutase (PGM): glucose 1-phosphate = glucose 6-phosphate

| | | | | | |
|---|---|---|---|---|---|
| 5.4.2.2 | PGM | Clostridium thermocellum | A3DEW8 | 578 | 118 |
| 5.4.2.2 | PGM | Thermus thermophilus | Q72H65 | 524 | 119 |

5.4.2.7. Phosphopentomutase (PPM): ribose 5-phopsphate (R5P) = ribose 1-phosphate (R1P)

| | | | | | |
|---|---|---|---|---|---|
| 5.4.2.7 | PPM | Thermotoga maritima | G4FH81 | 390 | 120 |
| 5.4.2.7 | PPM | Thermus thermophilus | Q72H36 | 381 | 121 |
| 5.4.2.7 | PPM | Clostridium thermocellum | A3DD83 | 388 | 122 |

The foregoing description is given for clearness of understanding only, and no unnecessary limitations should be understood therefrom, as modifications within the scope of the invention may be apparent to those having ordinary skill in the art.

The dimensions and values disclosed herein are not to be understood as being strictly limited to the exact numerical values recited. Instead, unless otherwise specified, each such dimension is intended to mean both the recited value and a functionally equivalent range surrounding that value. For example, a concentration disclosed as “10 mM” is intended to mean “about 10 mM.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making nicotinamide mononucleotide (NMN), NMN derivatives, or a mixture thereof, comprising generating a multi-enzyme reaction by mixing in an aqueous solution:
i. nicotinamide riboside phosphorylase and nicotinamide riboside kinase,
ii, nicotinamide,
iii, in vitro generated α-D-ribose-1-phosphate, α-D-ribose-1-phosphate derivatives, or a mixture thereof,
iv, an adenosine triphosphate (ATP) regeneration system, and
v. Mg²⁺,
wherein:
the nicotinamide riboside phosphorylase synthesizes nicotinamide riboside from nicotinamide and α-D-ribose-1-phosphate;
the nicotinamide riboside kinase synthesizes nicotinamide mononucleotide from nicotinamide riboside and ATP;
the ATP regeneration system synthesizes ATP from adenosine diphosphate (ADP) and inorganic orthophosphate anions; and
wherein byproducts of the multi-enzyme reaction are removed in-situ and converted to non-inhibitory chemicals with supplementary enzymes driving the multi-enzyme reaction toward the NMN synthesis.

2. The method of claim 1, wherein α-D-ribose-1-phosphate is generated from purine or pyrimidine nucleosides by purine nucleoside phosphorylase, guanosine nucleoside phosphorylase, pyrimidine-nucleoside phosphorylase, uridine phosphorylase, thymidine phosphorylase, or a mixture thereof and the purine or pyrimidine nucleosides comprise inosine, guanosine, adenosine, urdine, or thymidine.

3. The method of claim 1, wherein α-D-ribose-1-phosphate is produced from nucleotides via an intermediate α-D-ribose-5-diphosphate by (i) pyrimidine/purine nucleotide 5′-monophosphate nucleosidase, inosinate nucleosidase, or a mixture thereof, and followed by (ii) phosphopentomutase, the nucleotides comprising inosine monophosphate, guanosine monophosphate, or adenosine monophosphate.

4. The method of claim 1, wherein α-D-ribose-1-phosphate is produced from D-ribose via an intermediate α-D-ribose-5-diphosphate by (i) ribokinase and phosphopentomutase, and (ii) either an enzymatic ATP regeneration system or ATP-generating permeabilized living microorganisms, the ATP-generating permeabilized living microorganisms comprising Escherichia coli or Saccharomyces cerevisiae.

5. The method of claim 1, wherein the aqueous solution comprises the in vitro generated α-D-ribose-1-phosphate, α-D-ribose-1-phosphate derivatives, or a mixture thereof in an amount of 0.02 wt % or more.

6. A method of making nicotinamide mononucleotide (NMN), NMN derivatives, or a mixture thereof, comprising generating a multi-enzyme reaction by mixing in an aqueous solution:
i. hypoxanthine/guanine phosphoribosyltransferase and nicotinamide phosphoribosyltransferase,
ii, nicotinamide,
iii, inosine monophosphate, guanine monophosphate, or a mixture thereof,
iv. 5-phospho-alpha-D-ribose-1-diphosphate (PRPP),
v. inorganic diphosphate anions (PPᵢ), and
vi. Mg²⁺,
wherein:
the hypoxanthine/guanine phosphoribosyltransferase synthesizes PRPP and a purine base from nucleotides and PPᵢ, the nucleotides comprising inosine monophosphate or guanine monophosphate and the purine base comprising inosinate or guanine;
the nicotinamide phosphoribosyltransferase synthesizes nicotinamide mononucleotide from PRPP and nicotinamide, releasing PPᵢ to provide a substrate for the hypoxanthine/guanine phosphoribosyltransferase; and
wherein byproducts of the multi-enzyme reaction are removed in situ and converted to non-inhibitory chemicals with supplementary enzymes driving the multi-enzyme reaction toward the NMN synthesis.

7. The method of claim 6, wherein the guanine monophosphate provides guanine that is further converted to xanthine by guanine deaminase.

8. The method of claim 7, wherein the xanthine is converted to urate by xanthine oxidase and the enzyme catalase converts hydrogen peroxide to water and oxygen.

9. The method of claim 6, wherein:
inosine monophosphate provides hypoxanthine that is further converted to xanthine by xanthine oxidase, and/or
the enzyme catalase converts hydrogen peroxide to water and oxygen, and/or
xanthine dehydrogenase and the enzyme water-forming NAD(H) oxidase converts NADH to water and NAD⁺.

10. The method of claim 6, wherein the aqueous solution contains the PRPP in an amount of 0.02 wt % or more.

11. The method of claim 1, wherein nicotinamide adenine dinucleotide (NAD), one of the NMN derivatives, is synthesized from NMN and ATP by nicotinamide nucleotide adenylyltransferase.

12. The method of claim 11, wherein ATP is regenerated by an enzymatic ATP regeneration system or ATP-generating permeabilized living microorganisms, the ATP-generating permeabilized living microorganisms comprising Escherichia coli or Saccharomyces cerevisiae; and diphosphate is hydrolyzed by diphosphatase.

13. The method of claim 11, wherein nicotinamide adenine dinucleotide phosphate (NADP), one of the NMN derivatives, is synthesized from (i) NAD and (ii) ATP or polyphosphate by NAD kinase.

14. The method of claim 1, wherein nicotinamide riboside, one of the NMN derivatives, is hydrolyzed from NMN by 5′-nucleotidase.

15. The method of claim 1, wherein the pH of the aqueous solution is between about 3 and about 10.

16. The method of claim 1, wherein:
the temperature of the multi-enzyme reaction is between about 10 and about 80 degree of Celsius;
one or more cations of the multi-enzyme reaction are selected from the group consisting of Mg²⁺, Ca²⁺, Co²⁺, Mn²⁺, Zn²⁺, and a mixture thereof; and
one or more solvents of the multi-enzyme reaction comprise water.

17. The method of claim 1, wherein the multi-enzyme reaction comprises at least one recombinant polypeptide, the recombinant polypeptide having an amino acid sequence selected from a group consisting of SEQ ID NOs: 1-122 and produced from a recombinant vector which directs the expression of the polypeptide or its variant in a suitable expression host.

18. The method of claim 17, wherein the recombinant polypeptide is produced from strains Escherichia coli, Corynebacterum glutamicum, Aspergillus oryzae, or Bacillus subtilis.

19. The method of claim 17, wherein the recombinant polypeptide is produced from Escherichia coli BL21(DE3) and its derived strain harboring pET plasmid encoding the DNA sequence for the polypeptides of SEQ. IDs 1-122, said DNA sequence operably linked to a promoter to drive the expression of the polypeptides.

20. The method of claim 19, wherein the recombinant polypeptide is purified or immobilized and the multi-enzyme reaction comprises a crude cell lysate having said recombinant polypeptide, a whole-cell system producing said recombinant polypeptide, a permeabilized whole cell system containing said recombinant polypeptide, or a mixture thereof.
